(19) United States
(12) Patent Application Publication
(10) Pub. No.: US 2012/0141496 A1
(43) Pub. Date: Jun. 7, 2012

(54) IAP BIR DOMAIN BINDING COMPOUNDS

(75) Inventors: Alain Laurent, Montreal (CA); Scott Jarvis, Longueuil (CA)

(73) Assignee: AEGERA THERAPEUTICS, INC., Verdun, QC (CA)

(21) Appl. No.: 11/958,352

(22) Filed: Dec. 17, 2007

(51) Int. Cl. A61K 39/395; C07K 5/062; C07K 1/107; A61K 38/19; A61P 35/00; G01N 33/566; C12Q 1/02; C07K 5/103; A61K 38/07

(52) U.S. Cl. 424/158.1; 530/323; 548/540; 514/21.9; 514/19.3; 435/7.92; 435/29

(57) ABSTRACT

The present invention, according to several embodiments, comprises a compound represented by Formula I:

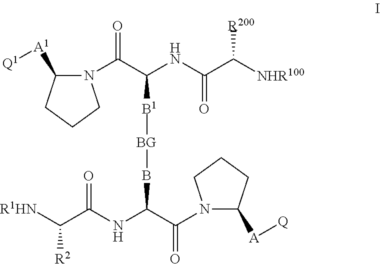

in which the substituents BG, B, B¹, R¹, R¹⁰⁰, R², R²⁰⁰, A, A¹, Q and Q¹ are defined herein; or a prodrug, or a salt thereof, and which bind to IAP BIR domains. In particular, the compounds of certain embodiments are useful in treating proliferative disorders such as cancer.

### BACKGROUND

1. Technical Field

The present disclosure is directed to compounds that bind to IAP BIR domains, and more particularly the BIR2 and BIR3 domains, and, in several embodiments, are useful to treat proliferative disorders.

2. Description of the Related Art

Apoptosis, or programmed cell death, typically occurs in the development and maintenance of healthy tissues in multicellular organisms. Apoptotic pathways are known to play a critical role in embryonic development, viral pathogenesis, cancer, autoimmune disorders, and neurodegenerative diseases, as well as other events. Alterations in an apoptotic response has been implicated in the development of cancer, autoimmune diseases, such as systemic lupus erythematosis and multiple sclerosis, and in viral infections, including those associated with herpes virus, poxvirus, and adenovirus.

Inhibitors of apoptosis proteins (IAPs) are a family of proteins, which contain one to three baculovirus IAP repeat (BIR) domains, namely BIR1, BIR2, and BIR3, and may also contain a RING zinc finger domain at the C-terminus. Examples of human IAPs include, XIAP, HIAP1 (also referred to as cIAP2), and HIAP2 (cIAP1) each have three BIR domains, and a carboxy terminal RING zinc finger. NAIP has three BIR domains (BIR1, BIR2 and BIR3), but no RING domain, whereas Livin and ILP2 have a single BIR domain and a RING domain. These proteins modulate various cellular apoptotic cascades. For example, caspases represent a class of cysteine proteases that are known to initiate apoptosis after they have been activated. The prototype X chromosome linked inhibitor of apoptosis (XIAP) inhibits activated caspases by direct binding to the caspases. XIAP can also remove caspases and the second mitochondrial activator of caspases (Smac) through the ubiquitylation-mediated proteasome pathway via the E3 ligase activity of a RING zinc finger domain. The BIR3 domain of XIAP binds and inhibits caspase-9, which can activate caspase-3. The linker-BIR2 domain of XIAP inhibits the activity of effector caspases-3 and -7. The BIR domains have also been associated with the interactions of IAPs with tumor necrosis factor-associated factor (TRAFs)-1 and -2, and to TAB1.

Overall the IAPs function as a “constraint” to apoptosis and may directly contribute to the tumor progression and resistance to pharmaceutical intervention. Interestingly, results demonstrate that resistance to apoptosis can be decrease by siRNA and antisense directed against specific IAP's in the cells; suggesting that interfering with the activity of the IAP's might prove advantageous in sensitizing disease cells to apoptosis.

A series of endogenous ligands are capable of interfering with IAP-caspase interactions. The X-ray crystallographic structure of XIAP BIR2 and BIR3 reveal a critical binding pocket and groove on the surface of each BIR domain. Two mammalian mitochondrial proteins, namely second mitochondria-derived activator of caspases (Smac) and Omi/Htra2, and four Drosophila proteins (Reaper, HID, Grim, and Sickle), which interfere with IAP function by binding to these sites on their respective BIR domain, have been identified. Each of these IAP binding proteins contain a short N-terminal tetrapeptide unit AXPY (AVPI-like sequence) that fits into this binding groove. Although the overall folding of individual BIR domains is generally conserved, there are alterations in the amino acid sequences that form the AXPX binding groove. As such, binding affinities vary between each of the BIR domains.

A number of compounds have been described, which reportedly bind XIAP including Wu et al., Chemistry and Biology, Vol. 10, 759-767 (2003); United States published patent application number US2006/0025347A1; United States published patent application number US2005/0197403A1; United States published patent application number US2006/0194741A1. Some of the aforesaid compounds, while they appear to target the BIR3 domain of XIAP, may have limited bioavailability and therefore limited therapeutic application. Moreover, the compounds may not be selective against other IAPs and indeed other BIR domains, such as BIR2; this lack of specificity may lead to unexpected side effects.

Thus, IAP BIR domains represent an attractive target for the discovery and development of novel therapeutic agents, especially for the treatment of proliferative disorders such as cancer.

### SUMMARY OF THE INVENTION

We have discovered a novel series of compounds that bind the IAPs and enhance cellular apoptosis through IAP modulation, and which have pharmaceutically acceptable stability and bioavailability. The compounds cause a reduction and/or loss of IAP proteins in cells before mitochondrial depolarization occurs and prevent the interaction of caspase 3, caspase 7, and caspase 9. Hence the results suggest that a small molecule is capable of down-regulating IAP proteins before cell death, thus indicating that clinically the use of the compounds may offer advantages when administered in combination with other inducers of apoptosis.

Specifically, we have demonstrated that the compounds bind to the BIR2 and BIR3 domain of mammalian XIAP and promote apoptosis of cancer cells as a single agent or in combination with a chemotherapeutic agent or a death receptor agonist, such as TRAIL or agonist TRAIL receptor antibodies. Advantageously, the compounds described herein have pro-apoptotic activity in various cancer cell lines such as bladder, breast, pancreatic, colon, leukemic, lung, lymphoma, multiple myloma and ovarian, and may also find application in other cancer cell lines and in diseases where cells are resistant to apoptosis. The compounds were found to kill cancer cells in a synergistic manner with TRAIL or with agonist TRAIL receptor anti-bodies. These results suggest that embodiments of compounds disclosed herein will demonstrate anti-cancer activity against solid tumors and tumors originating from the hematological malignancies. Moreover, the compounds of several embodiments may also find application in preventing cancer cell metastasis, invasion, inflammation, and in other diseases characterized by cells that are resistant to apoptosis. The compounds may also be useful in the treatment of autoimmune diseases.

In one embodiment, there is provided a compound represented by Formula I:

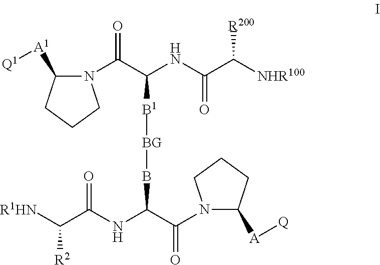

wherein
n is 0 or 1;
m is 0, 1 or 2;
p is 1 or 2;

### Y is NH, O or S;

B and B¹ are independently C₁-C₆ alkyl;

### BG is

1) -aryl-C₂-C₄ alkynyl-(X)ₙ—,
2) -aryl-(C₂-C₄ alkynyl)-aryl-,
3) biphenyl ether, or
4) biphenyl;

X is aryl or C₁-C₆ alkyl-O—;

R¹, R¹⁰⁰, R² and R²⁰⁰ are independently selected from:

### 1) H, or

2) C₁-C₆ alkyl optionally substituted with one or more R⁶ substituents;

Q and Q¹ are each independently

### 1) NR⁴R⁵,

### 2) OR¹¹, or

### 3) S(O)ₘR¹¹; or

Q and Q¹ are each independently

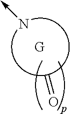

wherein G is a 5, 6 or 7 membered ring which optionally incorporates one or more heteroatoms chosen from S, N or O, the ring being optionally substituted with one or more R¹² substituents;

R⁴ and R⁵ are each independently

### H,

haloalkyl,
←C₁-C₆ alkyl,
←C₂-C₆ alkenyl,
←C₂-C₄ alkynyl,
←C₃-C₇ cycloalkyl,
←C₃-C₇ cycloalkenyl,
←aryl,
←heteroaryl,
←heterocyclyl,
←heterobicyclyl,

### ←C(O)—R¹¹,

### ←C(O)O—R¹¹,

### ←C(═Y)NR⁸R⁹, or

### ←S(O)₂—R¹¹,

wherein the alkyl, alkenyl, alkynyl, cycloalkyl, cycloalkenyl is optionally substituted with one or more R⁶ substituents; and wherein the aryl, heteroaryl, heterocyclyl, and heterobicyclyl is optionally substituted with one or more R¹⁰ substituents;

R⁶ is

1) halogen,

### 2) NO₂,

### 3) CN,

4) haloalkyl,
5) C₁-C₆ alkyl,
6) C₂-C₆ alkenyl,
7) C₂-C₄ alkynyl,
8) C₃-C₇ cycloalkyl,
9) C₃-C₇ cycloalkenyl,
10) aryl,
11) heteroaryl,
12) heterocyclyl,
13) heterobicyclyl,

### 14) OR⁷,

### 15) S(O)ₘR⁷,

### 16) NR⁸R⁹,

### 17) NR⁸S(O)₂R¹¹,

### 18) COR⁷,

### 19) C(O)OR⁷,

### 20) CONR⁸R⁹,

### 21) S(O)₂NR⁸R⁹

### 22) OC(O)R⁷,

### 23) OC(O)Y—R¹¹,

### 24) SC(O)R⁷, or

### 25) 1NC(Y)NR⁸R⁹,

wherein the aryl, heteroaryl, heterocyclyl, and heterobicyclyl is optionally substituted with one or more R¹⁰ substituents;

R⁷ is

1) H,

2) haloalkyl,

3) C₁-C₆ alkyl,

4) C₂-C₆ alkenyl,

5) C₂-C₄ alkynyl,

6) C₃-C₇ cycloalkyl,

7) C₃-C₇ cycloalkenyl,

8) aryl,

9) heteroaryl,

10) heterocyclyl,

11) heterobicyclyl,

12) R⁸R⁹NC(═Y), or

13) C₁-C₆ alkyl-C₂-C₄ alkenyl, or

14) C₁-C₆ alkyl-C₂-C₄ alkynyl,

wherein the alkyl, alkenyl, alkynyl, cycloalkyl, cycloalkenyl is optionally substituted with one or more R⁶ substituents; and wherein the aryl, heteroaryl, heterocyclyl, and heterobicyclyl is optionally substituted with one or more R¹⁰ substituents;

R⁸ and R⁹ are each independently

1) H,

2) haloalkyl,

3) C₁-C₆ alkyl,

4) C₂-C₆ alkenyl,

5) C₂-C₄ alkynyl,

6) C₃-C₇ cycloalkyl,

7) C₃-C₇ cycloalkenyl,

8) aryl,

9) heteroaryl,

10) heterocyclyl,

11) heterobicyclyl,

12) C(O)R¹¹,

13) C(O)Y—R¹¹, or

14) S(O)₂—R¹¹,

wherein the alkyl, alkenyl, alkynyl, cycloalkyl, cycloalkenyl is optionally substituted with one or more R⁶ substituents; and wherein the aryl, heteroaryl, heterocyclyl, and heterobicyclyl is optionally substituted with one or more R¹⁰ substituents;

or R⁸ and R⁹ together with the nitrogen atom to which they are bonded form a five, six or seven membered heterocyclic ring optionally substituted with one or more R⁶ substituents;

R¹⁰ is

1) halogen,

2) NO₂,

3) CN,

4) B(OR¹³)(OR¹⁴),

5) C₁-C₆ alkyl,

6) C₂-C₆ alkenyl,

7) C₂-C₄ alkynyl,

8) C₃-C₇ cycloalkyl,

9) C₃-C₇ cycloalkenyl,

10) haloalkyl,

11) OR⁷,

12)NR⁸R⁹,

13) SR⁷,

14) COR⁷,

15) C(O)O R⁷,

16) S(O)ₘR⁷,

17) CONR⁸R⁹,

18) S(O)₂NR⁸R⁹,

19) aryl,

20) heteroaryl,

21) heterocyclyl, or

22) heterobicyclyl,

wherein the alkyl, alkenyl, alkynyl, cycloalkyl, and cycloalkenyl is optionally substituted with one or more R⁶ substituents;

R¹¹ is

1) haloalkyl,

2) C₁-C₆ alkyl,

3) C₂-C₆ alkenyl,

4) C₂-C₄ alkynyl,

5) C₃-C₇ cycloalkyl,

6) C₃-C₇ cycloalkenyl,

7) aryl,

8) heteroaryl,

9) heterocyclyl, or

10) heterobicyclyl,

wherein the alkyl, alkenyl, alkynyl, cycloalkyl, cycloalkenyl is optionally substituted with one or more R⁶ substituents; and wherein the aryl, heteroaryl, heterocyclyl, and heterobicyclyl is optionally substituted with one or more R¹⁰ substituents;

R¹² is

1) haloalkyl,

2) C₁-C₆ alkyl,

3) C₂-C₆ alkenyl,

4) C₂-C₄ alkynyl,

5) C₃-C₇ cycloalkyl,

6) C₃-C₇ cycloalkenyl,

7) aryl,

8) heteroaryl,

9) heterocyclyl,

10) heterobicyclyl,

11) C(O)—R¹¹,

12) C(O)O—R¹¹,

13) C(O)NR⁸R⁹,

14) S(O)ₘ—R¹¹, or

15) C(═Y)NR⁸R⁹,

wherein the alkyl, alkenyl, alkynyl, cycloalkyl, cycloalkenyl is optionally substituted with one or more R⁶ substituents; and wherein the aryl, heteroaryl, heterocyclyl, and heterobicyclyl is optionally substituted with one or more R¹⁰ substituents;

R¹³ and R¹⁴ are each independently 1) H, or 2) C₁-C₆ alkyl; or R¹³ and R¹⁴ are combined to form a heterocyclic ring or a heterobicyclyl ring; or a prodrug; or the compound of Formula I is labeled with a detectable label or an affinity tag.

In another aspect, there is provided an intermediate compound represented by Formula 2(iii):

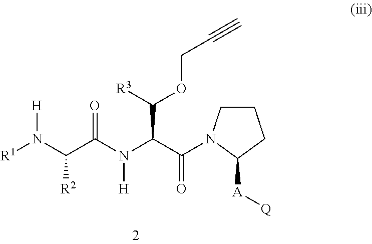

wherein R¹, R², R³, A, and Q are as defined herein.

In another aspect, there is provided an intermediate compound represented by Formula 6(i):

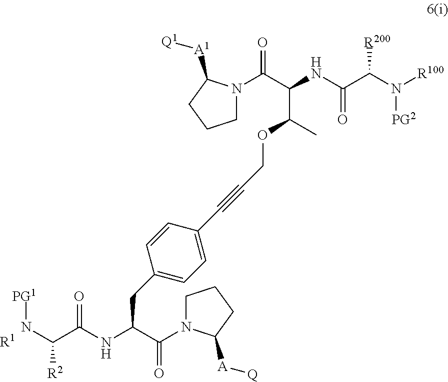

wherein PG¹ and PG² are protecting groups and R¹, R², R³, A, Q, R¹⁰⁰, R²⁰⁰, A¹ and Q¹ are as defined herein.

In another aspect, there is provided an intermediate compound represented by Formula 7(i):

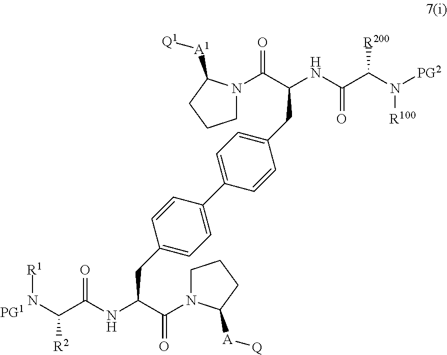

wherein PG¹ and PG² are protecting groups and R¹, R², R³, A, Q, R¹⁰⁰, R²⁰⁰, A¹ and Q¹ are as defined herein.

In another aspect, there is provided an intermediate compound represented by Formula 8(i):

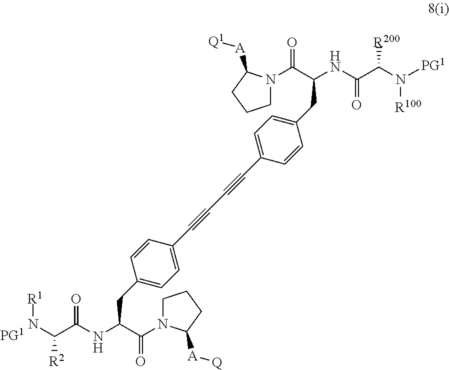

wherein PG¹ is a protecting group, and R¹, R², R³, A, Q, R¹⁰⁰, R²⁰⁰, A¹ and Q¹ are as defined herein.

In another aspect, there is provided a process for producing compounds of Formula 6(ii), the process comprising:

a) deprotecting PG¹ and PG² from intermediate 6(i):

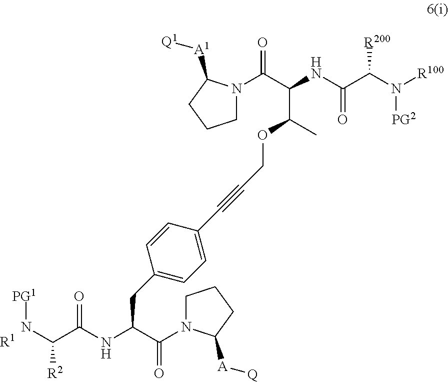

so as to produce compounds of Formula 6(ii):

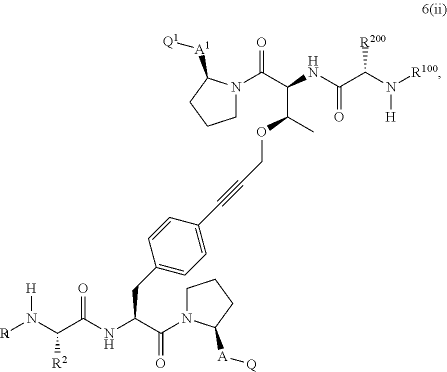

wherein PG¹ and PG² are protecting groups and R¹, R², R³, A, Q, R¹⁰⁰, R²⁰⁰, A¹ and Q¹ are as defined herein.

In another aspect, there is provided a process for producing compounds of Formula 7(ii), the process comprising:

a) deprotecting PG¹ and PG² from intermediate 7(i):

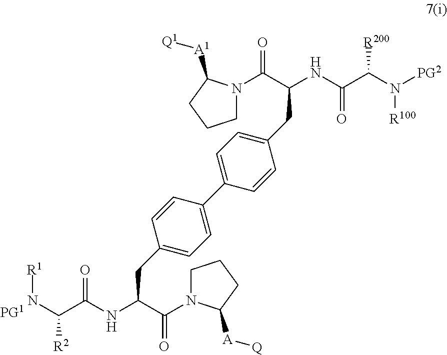

so as to produce compounds of Formula 7(ii):

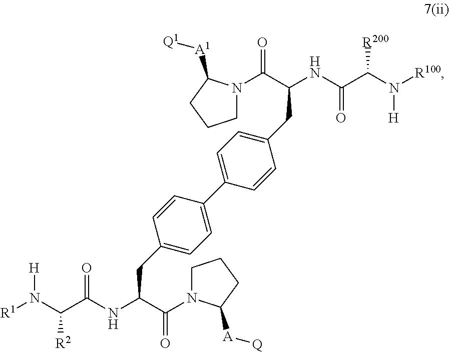

wherein PG¹ and PG² are protecting groups and R¹, R², R³, A, Q, R¹⁰⁰, R²⁰⁰, A¹ and Q¹ are as defined herein.

In another aspect, there is provided a process for producing compounds of Formula 8(ii) above, the process comprising:

a) deprotecting PG¹ and PG² from intermediate 8(i):

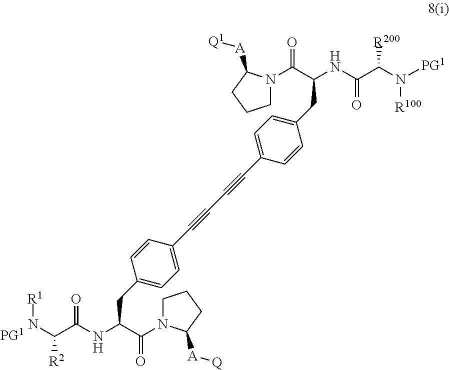

so as to produce compounds of Formula 8(iii):

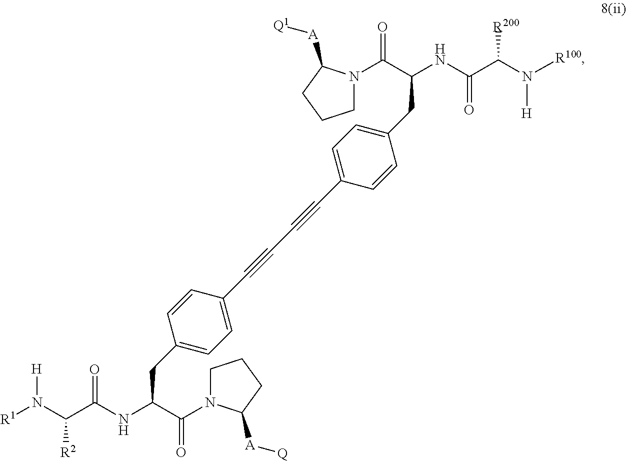

wherein PG¹ and PG² are protecting groups and R¹, R², R³, A, Q, R¹⁰⁰, R²⁰⁰, A¹ and Q¹ are as defined herein.

In another aspect, there is provided a pharmaceutical composition comprising the compound of Formula I, as described above, and a pharmaceutically acceptable carrier, diluent or excipient.

In another aspect, there is provided a pharmaceutical composition comprising the compound of Formula I, as described above, in combination with any compound that increases the circulating level of one or more death receptor agonists for preventing or treating a proliferative disorder.

In another aspect, there is provided a method for the preparation of a pharmaceutically acceptable salt of compound of Formula I, as described above, by the treatment of a compound of Formula I with 1 to 2 equiv of a pharmaceutically acceptable acid.

In another aspect, there is provided a method for the treatment or prevention of a proliferative disorder in a subject, the method comprising: administering to the subject a therapeutically effective amount of the compound or a salt thereof, as described above. In one example, the proliferative disorder is cancer. The method above further comprising administering to the subject a therapeutically effective amount of a chemotherapeutic agent prior to, simultaneously with or after administration of the compound. The method above further comprising administering to the subject a therapeutically effective amount of a death receptor agonist prior to, simultaneously with or after administration of the compound. In one example, the death receptor agonist is TRAIL.

In another example, the death receptor agonist is a TRAIL antibody. The death receptor agonist is administered in an amount that produces a synergistic effect. In one example, the subject is a human.

In another aspect, there is provided a method for the treatment or prevention of cancer in a subject, the method comprising: administering to the subject a therapeutically effective amount of the pharmaceutical composition, as described above so as to treat or prevent the cancer. In one example, the subject is a human.

In another aspect, there is provided a method of detecting loss of function or suppression of IAPs in vivo, the method comprising: a) administering to a subject, a therapeutically effective amount of a pharmaceutical composition, as described above; b) isolating a tissue sample from the subject; and c) detecting a loss of function or suppression of IAPs from the sample. The IAP is XIAP, c-IAP1 or c-IAP2. The tissue is a tumor sample or normal tissue. The tissue is isolated from the hematopeotic system. In one example, mononuclear cells are isolated from blood, and IAP suppression or loss is measured in the mononuclear cells. In another example, the salt of the compound is administered intravenously.

In another aspect, there is provided a probe, the probe being a compound of Formula I above, the compound being labeled with a detectable label or an affinity tag.

In another aspect, there is provided a method of identifying compounds that bind to an IAP BIR domain, the assay comprising:
a) contacting an IAP BIR domain with a probe to form a probe: BIR domain complex, the probe being displaceable by a test compound;
b) measuring a signal from the probe so as to establish a reference level;
c) incubating the probe: BIR domain complex with the test compound;
d) measuring the signal from the probe;
e) comparing the signal from step d) with the reference level, a modulation of the signal being an indication that the test compound binds to the BIR domain,
   wherein the probe is a compound of Formula I labeled with a detectable label or an affinity label.

### DETAILED DESCRIPTION OF THE PREFERRED EMBODIMENT

In many cancer and other diseases, an up-regulation of XIAP induced by gene defects or by chemotherapeutic agents has been correlated to an increased resistance to apoptosis. Interestingly our results show that cells decreased in XIAP level are more sensitive to TRAIL induced apoptosis. It is believed that a small molecule, which will induce XIAP loss from disease cells, will be useful as a therapeutic agent. We report herein compounds that can directly bind to XIAP, cause a down regulation of the XIAP protein in cell before cell death, induce apoptosis in cancer cells, and have a synergistic effect in combination with inducers of apoptosis. This would provide clinical advantages in terms of the selectivity of therapy based on the phenotype of the cancer cells. Also advantageous according to several embodiments would be the use of one or more of the compounds disclosed herein in combination therapy with other agents in terms of the doses of administration and the time of scheduling the doses.

The compounds of several embodiments are useful as BIR domain binding compounds in mammalian IAPs and are represented by Formula I:

### A and A¹:

In one subset of compounds of Formula 1, A and A¹ are both CH₂.

In an alternative subset of compounds of Formula 1, A and A¹ are both C═O.

In another alternative subset of compounds of Formula 1, A is CH₂ and A¹ is C═O.

Any and each individual definition of A and A¹ as set out herein may be combined with any and each individual definition of Core, R¹, R², R¹⁰⁰, R²⁰⁰, Q, Q¹, B, B¹, and BG as set out herein.

### Core:

Therefore, several embodiments comprise compounds of Formula 1a through 1c:

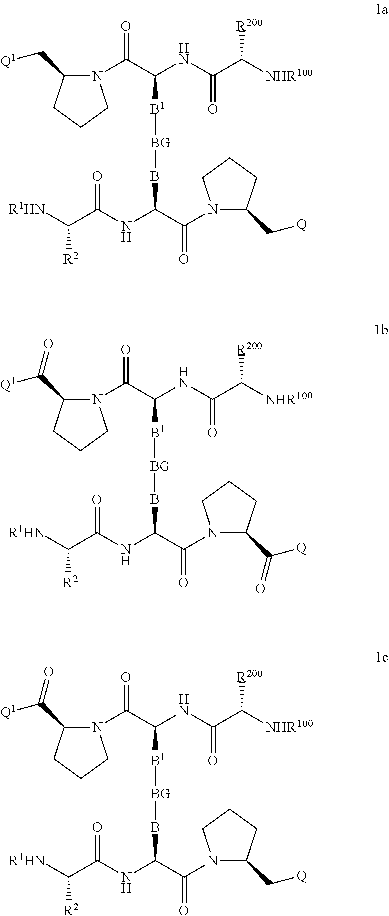

wherein BG, B, B¹, Q, Q¹, R¹, R¹⁰⁰, R² and R²⁰⁰ are as defined hereinabove and hereinafter.

In one example, several embodiments comprise compounds of Formula 1a.

In an alternative example, several embodiments comprise compounds of Formula 1b.

Any and each individual definition of Core as set out herein may be combined with any and each individual definition of A, A¹, R¹, R², R¹⁰⁰, R²⁰⁰, Q, Q¹, B, B¹, and BG as set out herein.

### B and B¹:

In one subset of the aforesaid compounds, B and B¹ are both C₁-C₄ alkyl.

Any and each individual definition of B and B¹ as set out herein may be combined with any and each individual definition of Core, A, A¹, R¹, R², R¹⁰⁰, R²⁰⁰, Q, Q¹, and BG as set out herein.

### BG:

In one aspect, BG is -aryl-C₂-C₄ alkynyl-(X)ₙ—.

Therefore several embodiments comprise compounds of Formula 1d and 1e:

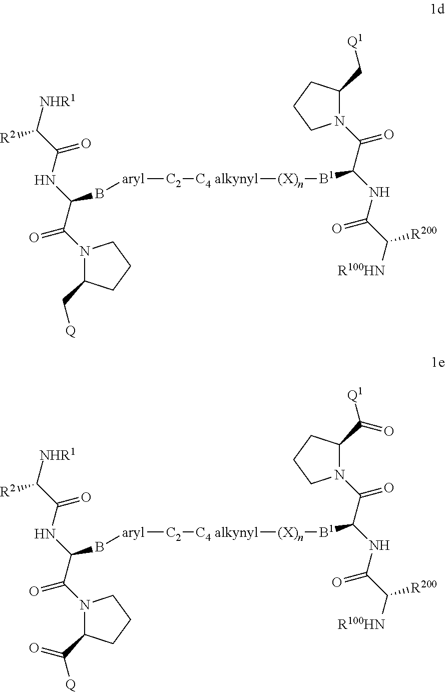

wherein X, n, B, B¹, Q, Q¹, R¹, R¹⁰⁰, R² and R²⁰⁰ are as defined hereinabove and hereinafter.

In an alternative aspect, BG is biphenyl ether, or biphenyl. Therefore several embodiments comprise compounds of Formula 1f and 1 g:

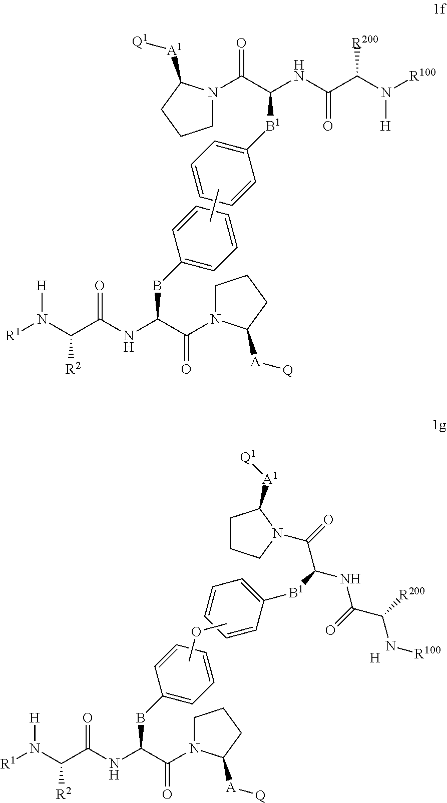

Any and each individual definition of BG as set out herein may be combined with any and each individual definition of Core, B, B¹, A, A¹, R¹, R², R¹⁰⁰, R²⁰⁰, Q, and Q¹ as set out herein.

### X:

In one subset of the aforesaid compounds, X is aryl. One typical example of X is phenyl.

In an alternative subset of the aforesaid compounds, X is C₁-C₆ alkyl-O—.

Thus more explicitly, BG is

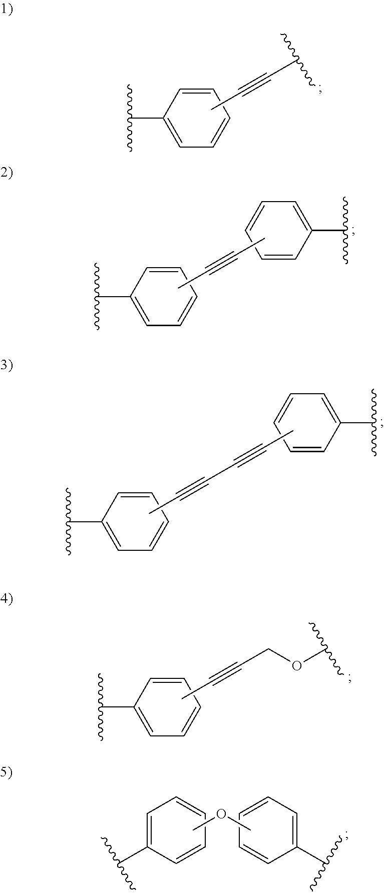

or
6) biphenyl.

Therefore the some embodiments comprise compounds of Formula 1 h, 1i, 1j, 1k, 1l and 1m:

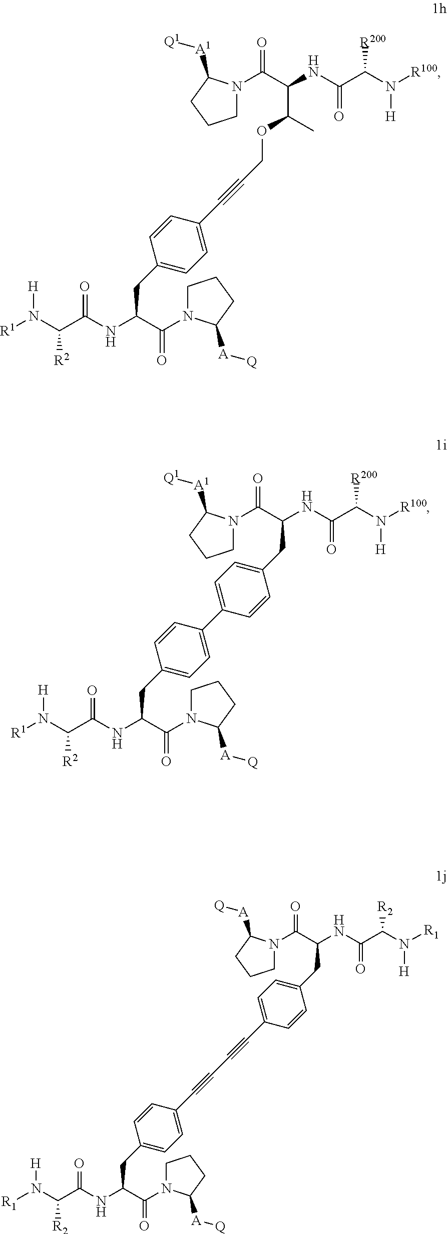

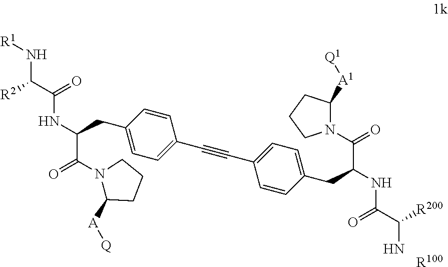

### R¹ and R¹⁰⁰:

In one subset of the aforesaid compounds R¹ and R¹⁰⁰ are both C₁-C₆ alkyl.

In one example, R¹ and R¹⁰⁰ are both CH₃.

Any and each individual definition of R₁ and R¹⁰⁰ as set out herein may be combined with any and each individual definition of Core, A, A¹, R², R²⁰⁰, Q, Q¹, B, B¹, and BG as set out herein.

### R² and R²⁰⁰:

In one subset of the aforesaid compounds R² and R²⁰⁰ are both C₁-C₆ alkyl.

In one example, R² and R²⁰⁰ are both CH₃.

Any and each individual definition of R² and R²⁰⁰ as set out herein may be combined with any and each individual definition of Core, A, A¹, R¹, R¹⁰⁰, Q, Q¹, B, B¹, and BG as set out herein.

### Q and Q¹:

In one subset of the aforesaid compounds, Q and Q¹ are both NR⁴R⁵, wherein R⁴ and R⁵ are as defined herein.

Any and each individual definition of Q and Q¹ as set out herein may be combined with any and each individual definition of Core, A, A¹, R¹, R¹⁰⁰, R², R²⁰⁰, B, B¹, and BG as set out herein.

### R⁴ and R⁵:

In one subset of the aforesaid compounds in which A and A¹ are both C═O, R⁴ is H and

R⁵ is selected from:

1) haloalkyl,

2) C₁-C₆ alkyl,

3) C₂-C₆ alkenyl,

4) C₂-C₄ alkynyl,

5) C₃-C₇ cycloalkyl,

6) C₃-C₇ cycloalkenyl,

7) aryl,

8) heteroaryl,

9) heterocyclyl, or

10) heterobicyclyl,

wherein the alkyl, alkenyl, alkynyl, cycloalkyl, cycloalkenyl is optionally substituted with one or more R⁶ substituents; and wherein the aryl, heteroaryl, heterocyclyl, and heterobicyclyl is optionally substituted with one or more R¹⁰ substituents;
wherein R⁶ and R¹⁰ are as defined herein.

In an alternative subset of the aforesaid compounds in which A and A¹ are both CH₂, then R⁴ and R⁵ are each independently:

1) H,

2) haloalkyl,

3) C₁-C₆ alkyl,

4) C₂-C₆ alkenyl,

5) C₂-C₄ alkynyl,

6) C₃-C₇ cycloalkyl,

7) C₃-C₇ cycloalkenyl,

8) aryl,

9) heteroaryl,

10) heterocyclyl,

11) heterobicyclyl,

12) C(O)—R¹¹,

13) C(O)O—R¹¹,

13) C(═Y)NR⁸R⁹, or

14) S(O)₂—R¹¹,

wherein the alkyl, alkenyl, alkynyl, cycloalkyl, cycloalkenyl is optionally substituted with one or more R⁶ substituents; and wherein the aryl, heteroaryl, heterocyclyl, and heterobicyclyl is optionally substituted with one or more R¹⁰ substituents; wherein Y, R⁶, R⁸, R⁹, R¹⁰ and R¹¹ are as defined herein.

Any and each individual definition of R⁴ and R⁵ as set out herein may be combined with any and each individual definition of Core, A, A¹, R¹, R¹⁰⁰, R², R²⁰⁰, B, B¹, and BG as set out herein.

### R¹¹:

In one subset of the aforesaid compounds,

### R¹¹ is:

1) haloalkyl,

2) C₁-C₆ alkyl,

3) C₂-C₆ alkenyl,

4) C₂-C₄ alkynyl,

5) aryl,

6) heteroaryl,

7) heterocyclyl, or

8) heterobicyclyl,

wherein the alkyl, alkenyl, alkynyl is optionally substituted with one or more R⁶ substituents; and wherein the aryl, heteroaryl, heterocyclyl, and heterobicyclyl is optionally substituted with one or more R¹⁰ substituents; wherein R⁶ and R¹⁰ are as defined herein.

Any and each individual definition of R¹¹ as set out herein may be combined with any and each individual definition of Core, A, A¹, R¹, R¹⁰⁰, R², R²⁰⁰, R⁴, R⁵, B, B¹, and BG as set out herein.

### R⁶:

In one subset of the aforesaid compounds, R⁶ is

1) halogen,

2) NO₂,

3) CN,

4) aryl,

5) heteroaryl,

6) heterocyclyl,

7) heterobicyclyl,

8) OR⁷,

9) SR⁷, or

10) NR⁸R⁹,

wherein the aryl, heteroaryl, heterocyclyl, and heterobicyclyl is optionally substituted with one or more R¹⁰ substituents; wherein R⁷, R⁸, R⁹ and R¹⁰ are as defined herein.

Any and each individual definition of R⁶ as set out herein may be combined with any and each individual definition of Core, A, A, A¹, R¹, R¹⁰⁰, R², R²⁰⁰, R⁴, R⁵, B, B¹, and BG as set out herein.

### R⁸ and R⁹:

In one subset of the aforesaid compounds, R⁸ and R⁹ are each independently:

1) H,

2) haloalkyl,

3) C₁-C₆ alkyl,

4) C₂-C₆ alkenyl,

5) C₂-C₄ alkynyl,

6) C₃-C₇ cycloalkyl, or

7) C₃-C₇ cycloalkenyl,

wherein the alkyl, alkenyl, alkynyl, cycloalkyl, cycloalkenyl is optionally substituted with one or more R⁶ substituents; wherein the R⁶ substituents are as defined herein.

Any and each individual definition of R⁸ and R⁹ as set out herein may be combined with any and each individual definition of Core, A, A¹, R¹, R¹⁰⁰, R², R²⁰⁰, R⁴, R⁵, B, B¹, and BG as set out herein.

### R¹⁰:

In one aspect of the aforesaid compounds, R¹⁰ is:

1) halogen,

2) NO₂,

3) CN,

4) haloalkyl,

5) OR⁷,

6) NR⁸R⁹, or

7) SR⁷;

wherein R⁷, R⁸, and R⁹ are as defined herein.

Any and each individual definition of R¹⁰ as set out herein may be combined with any and each individual definition of Core, A, A¹, R¹, R¹⁰⁰, R², R²⁰⁰, R⁴, R⁵, B, B¹, and BG as set out herein.

In one aspect, embodiments of the compounds may also be represented by Formula 2 in which M1 and M2 represent independent BIR binding domains.

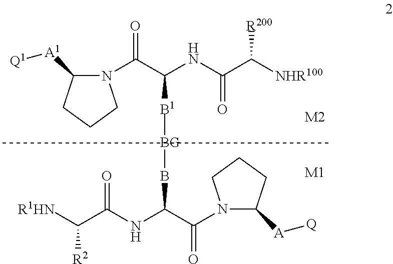

wherein R¹, R², R¹⁰⁰, R²⁰⁰, A, A¹, Q, Q¹, B, B¹, and BG as defined herein and the dotted line represents a hypothetical dividing line for comparing the substituents associated with M1 and M2.

In one subset of compounds of Formula 2, M1 is the same as M2.

In an alternative subset of compounds of Formula 2, M1 is different from M2.

In still another subset, B is the same as B¹.

In still another subset B is different from B¹.

One skilled in the art will recognize that when M1 and M2 are the same, the R¹, R², R⁴, R⁵, R⁶, R⁷, R⁸, R⁹, R¹⁰, R¹¹, R¹³, R¹⁴, m, p, Y, A, Q, and B substituents in M1 have the same meaning as the R¹⁰⁰, R²⁰⁰, R⁴, R⁵, R⁶, R⁷, R⁸, R⁹, R¹⁰, R¹¹, R¹³, R¹⁴, m, p, A¹, Q¹, and B¹ substituents respectively in M2. When M1 and M2 are different, at least one R¹, R², R¹⁰⁰, R²⁰⁰, R⁴, R⁵, R⁶, R⁷, R⁸, R⁹, R¹⁰, R¹¹, R¹³, R¹⁴, m, p, Y, A, A¹, Q, Q¹, B, and B¹ substituent is different in either of M1 or M2.

Alternatively the substituents in M1 can be defined as R¹, R², R⁴, R⁵, R⁶, R⁷, R⁸, R⁹, R¹⁰, R¹¹, R¹³, R¹⁴, m, p, Y, A, Q, and B, and those in M2 can be defined as R¹⁰⁰, R²⁰⁰, R⁴⁰⁰, R⁵⁰⁰, R⁶⁰⁰, R⁷⁰⁰, R⁸⁰⁰, R⁹⁰⁰, R¹⁰⁰⁰, R¹¹⁰⁰, R¹³⁰⁰, R¹⁴⁰⁰, m¹, p¹, Y¹, A¹, Q¹ and B¹ respectively. In the case where M1 and M2 are the same, the R¹, R², R⁴, R⁵, R⁶, R⁷, R⁸, R⁹, R¹⁰, R¹¹, R¹³, R¹⁴, m, p, Y, A, Q, and B substituents in M1 have the same meanings as R¹⁰⁰, R²⁰⁰, R⁴⁰⁰, R⁵⁰⁰, R⁶⁰⁰, R⁷⁰⁰, R⁸⁰, R⁹⁰⁰, R¹⁰⁰⁰, R¹¹⁰⁰, R¹³⁰⁰, R¹⁴⁰⁰, m¹, p¹, Y¹, A¹, Q¹ and B¹ respectively in M2. In the case where M1 and M2 are different, at least one of the aforesaid substituents is different.

If any variable, such as R⁶, R⁶⁰⁰, R¹⁰, R¹⁰⁰⁰ and the like, occurs more than one time in any constituent structure, the definition of the variable at each occurrence is independent at every other occurrence. If a substituent is itself substituted with one or more substituents, it is to be understood that that the one or more substituents may be attached to the same carbon atom or different carbon atoms. Combinations of substituents and variables defined herein are allowed only if they produce chemically stable compounds.

One skilled in the art will understand that substitution patterns and substituents on compounds of embodiments may be selected to provide compounds that are chemically stable and can be readily synthesized using the chemistry set forth in the examples and chemistry techniques well known in the art using readily available starting materials and reagents.

It is to be understood that many substituents or groups described herein have functional group equivalents, which means that the group or substituent may be replaced by another group or substituent that has similar electronic, hybridization or bonding properties.

### DEFINITIONS

Unless otherwise specified, the following definitions apply:

The singular forms “a”, “an” and “the” include corresponding plural references unless the context clearly dictates otherwise.

As used herein, the term “comprising” is intended to mean that the list of elements following the word “comprising” are required or mandatory but that other elements are optional and may or may not be present.

As used herein, the term “consisting of” is intended to mean including and limited to whatever follows the phrase “consisting of”. Thus the phrase “consisting of” indicates that the listed elements are required or mandatory and that no other elements may be present.

As used herein, the term “alkyl” is intended to include both branched and straight chain saturated aliphatic hydrocarbon groups having the specified number of carbon atoms, for example, C₁-C₁₀ as in C₁-C₁₀ alkyl is defined as including groups having 1, 2, 3, 4, 5, 6, 7, 8, 9 or 10 carbons in a linear or branched arrangement, and C₁-C₆ as in C₁-C₆— alkyl is defined as including groups having 1, 2, 3, 4, 5 or 6 carbons in a linear or branched arrangement, and C₁-C₄ as in C₁-C₄ alkyl is defined as including groups having 1, 2, 3, or 4 carbons in a linear or branched arrangement. Examples of C₁-C₆-alkyl and C₁-C₄ alkyl as defined above include, but are not limited to, methyl, ethyl, n-propyl, i-propyl, n-butyl, t-butyl, i-butyl, pentyl and hexyl.

As used herein, the term, “alkenyl” is intended to mean unsaturated straight or branched chain hydrocarbon groups having the specified number of carbon atoms therein, and in which at least two of the carbon atoms are bonded to each other by a double bond, and having either E or Z regeochemistry and combinations thereof. For example, C₂-C₆ as in C₂-C₆ alkenyl is defined as including groups having 1, 2, 3, 4, 5, or 6 carbons in a linear or branched arrangement, at least two of the carbon atoms being bonded together by a double bond. Examples of C₂-C₆ alkenyl include ethenyl (vinyl), 1-propenyl, 2-propenyl, 1-butenyl and the like.

As used herein, the term “alkynyl” is intended to mean unsaturated, straight chain hydrocarbon groups having the specified number of carbon atoms therein and in which at least two carbon atoms are bonded together by a triple bond. For example C₂-C₄ as in C₂-C₄ alkynyl is defined as including groups having 2, 3, or 4 carbon atoms in a chain, at least two of the carbon atoms being bonded together by a triple bond. Examples of such alynyls include ethynyl, 1-propynyl, 2-propynyl and the like.

As used herein, the term “cycloalkyl” is intended to mean a monocyclic saturated aliphatic hydrocarbon group having the specified number of carbon atoms therein, for example, C₃-C₇ as in C₃-C₇ cycloalkyl is defined as including groups having 3, 4, 5, 6, or 7 carbons in a monocyclic arrangement. Examples of C₃-C₇ cycloalkyl as defined above include, but are not limited to, cyclopropyl, cyclobutyl, cyclopentyl, cyclohexyl and cycloheptyl.

As used herein, the term “cycloalkenyl” is intended to mean a monocyclic saturated aliphatic hydrocarbon group having the specified number of carbon atoms therein, for example, C₃-C₇ as in C₃-C₇ cycloalkenyl is defined as including groups having 3, 4, 5, 6, or 7 carbons in a monocyclic arrangement. Examples of C₃-C₇ cycloalkenyl as defined above include, but are not limited to, cyclopentenyl, and cyclohexenyl.

As used herein, the term “halo” or “halogen” is intended to mean fluorine, chlorine, bromine and iodine.

As used herein, the term “haloalkyl” is intended to mean an alkyl as defined above, in which each hydrogen atom may be successively replaced by a halogen atom. Examples of haloalkyls include, but are not limited to, CH₂F, CHF₂ and CF₃.

As used herein, the term “aryl”, either alone or in combination with another radical, means a carbocyclic aromatic monocyclic group containing 6 carbon atoms which may be further fused to a second 5- or 6-membered carbocyclic group which may be aromatic, saturated or unsaturated. Aryl includes, but is not limited to, phenyl, indanyl, 1-naphthyl, 2-naphthyl and tetrahydronaphthyl. The fused aryls may be connected to another group either at a suitable position on the cycloalkyl ring or the aromatic ring. For example:

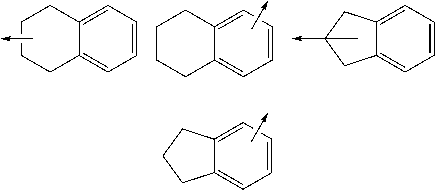

Arrowed lines drawn from the ring system indicate that the bond may be attached to any of the suitable ring atoms.

As used herein, the term “biphenyl” is intended to mean two phenyl groups bonded together at any one of the available sites on the phenyl ring. The biphenyl may be covalently bonded to other groups from any available position on the phenyl rings. For example:

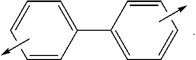

As used herein, the term “heteroaryl” is intended to mean a monocyclic or bicyclic ring system of up to ten atoms, wherein at least one ring is aromatic, and contains from 1 to 4 hetero atoms selected from the group consisting of O, N, and S. The heteroaryl substituent may be attached either via a ring carbon atom or one of the heteroatoms. Examples of heteroaryl groups include, but are not limited to thienyl, benzimidazolyl, benzo[b]thienyl, furyl, benzofuranyl, pyranyl, isobenzofuranyl, chromenyl, xanthenyl, 2H-pyrrolyl, pyrrolyl, imidazolyl, pyrazolyl, pyridyl, pyrazinyl, pyrimidinyl, pyridazinyl, indolizinyl, isoindolyl, 3H-indolyl, indolyl, indazolyl, purinyl, 4H-quinolizinyl, isoquinolyl, quinolyl, phthalazinyl, napthyridinyl, quinoxalinyl, quinazolinyl, cinnolinyl, pteridinyl, isothiazolyl, isochromanyl, chromanyl, isoxazolyl, furazanyl, indolinyl, isoindolinyl, thiazolo[4,5-b]-pyridine, and fluoroscene derivatives such as:

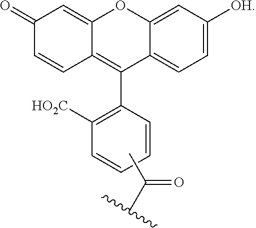

As used herein, the term “heterocycle”, “heterocyclic” or “heterocyclyl” is intended to mean a 5, 6, or 7 membered non-aromatic ring system containing from 1 to 4 heteroatoms selected from the group consisting of O, N and S. Examples of heterocycles include, but are not limited to pyrrolidinyl, tetrahydrofuranyl, piperidyl, pyrrolinyl, piperazinyl, imidazolidinyl, morpholinyl, imidazolinyl, pyrazolidinyl, pyrazolinyl, and

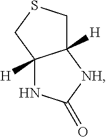

As used herein, the term “heterobicycle” either alone or in combination with another radical, is intended to mean a heterocycle as defined above fused to another cycle, be it a heterocycle, an aryl or any other cycle defined herein. Examples of such heterobicycles include, but are not limited to, coumarin, benzo[d][1,3]dioxole, 2,3-dihydrobenzo[b][1,4]dioxine and 3,4-dihydro-2H-benzo[b][1,4]dioepine.

### Examples of

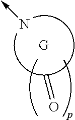

wherein G is a 5, 6 or 7 membered ring which optionally incorporates one or more heteroatoms selected from S, N or O and p is 1 or 2, and is optionally substituted with one or more R¹² substituents, include, but are not limited to:

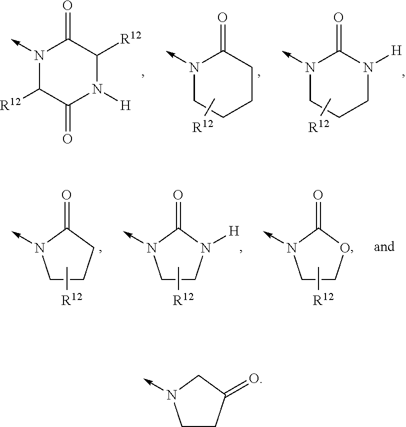

As used herein, the term “heteroatom” is intended to mean O, S or N.

As used herein, the term “detectable label” is intended to mean a group that may be linked to a compound of embodiments to produce a probe or to an IAP BIR domain, such that when the probe is associated with the BIR domain, the label allows either direct or indirect recognition of the probe so that it may be detected, measured and quantified.

As used herein, the term “affinity tag” is intended to mean a ligand or group, which is linked to either a compound of embodiments or to an IAP BIR domain to allow another compound to be extracted from a solution to which the ligand or group is attached.

As used herein, the term “probe” is intended to mean a compound of Formula I which is labeled with either a detectable label or an affinity tag, and which is capable of binding, either covalently or non-covalently, to an IAP BIR domain. When, for example, the probe is non-covalently bound, it may be displaced by a test compound. When, for example, the probe is bound covalently, it may be used to form cross-linked adducts, which may be quantified and inhibited by a test compound.

As used herein, the term “optionally substituted with one or more substituents” or its equivalent term “optionally substituted with at least one substituent” is intended to mean that the subsequently described event of circumstances may or may not occur, and that the description includes instances where the event or circumstance occurs and instances in which it does not. The definition is intended to mean from zero to five substituents.

If the substituents themselves are incompatible with the synthetic methods of embodiments, the substituent may be protected with a suitable protecting group (PG) that is stable to the reaction conditions used in these methods. The protecting group may be removed at a suitable point in the reaction sequence of the method to provide a desired intermediate or target compound. Suitable protecting groups and the methods for protecting and de-protecting different substituents using such suitable protecting groups are well known to those skilled in the art; examples of which may be found in T. Greene and P. Wuts, Protecting Groups in Chemical Synthesis (3^{rd} ed.), John Wiley & Sons, NY (1999), which is incorporated herein by reference in its entirety. Examples of protecting groups used throughout include, but are not limited to Fmoc, Bn, Boc, CBz and COCF₃. In some instances, a substituent may be specifically selected to be reactive under the reaction conditions used in some embodiments of the methods. Under these circumstances, the reaction conditions convert the selected substituent into another substituent that is either useful in an intermediate compound in embodiments of the methods or is a desired substituent in a target compound.

Abbreviations for α-amino acids used throughout are as follows:

| Amino acid | Abbreviation |
|---|---|
| α-Amino butyric acid | Abu |
| Alanine | Ala |
| Arginine | Arg |
| Aspartic acid | Asp |
| Asparagine | Asn |
| Cysteine | Cys |
| Glutamic acid | Glu |
| Glutamine | Gln |
| Glycine | Gly |
| Isoleucine | Ile |
| Histidine | His |
| Leucine | Leu |
| Lysine | Lys |
| Methionine | Met |
| Phenylalanine | Phe |
| Proline | Pro |
| Serine | Ser |
| Threonine | Thr |
| Tryptophan | Trp |
| Tyrosine | Tyr |
| Valine | Val |

As used herein, the term “residue” when referring to α-Amino is intended to mean a radical derived from the corresponding α-amino by eliminating the hydroxyl of the carboxy group and one hydrogen of the α-amino. For example, the terms Gln, Ala, Gly, Ile, Arg, Asp, Phe, Ser, Leu, Cys, Asn, and Tyr represent the residues of L-glutamine, L-alanine, glycine, L-isoleucine, L-arginine, L-aspartic acid, L-phenylalanine, L-serine, L-leucine, L-cysteine, L-asparagine, and L-tyrosine, respectively.

As used herein, the term “subject” is intended to mean humans and non-human mammals such as primates, cats, dogs, swine, cattle, sheep, goats, horses, rabbits, rats, mice and the like.

As used herein, the term “prodrug” is intended to mean a compound that may be converted under physiological conditions or by solvolysis to a biologically active compound of some embodiments. Thus, the term “prodrug” refers to a precursor of an embodiment of a compound that is pharmaceutically acceptable. A prodrug may be inactive or display limited activity when administered to a subject in need thereof, but is converted in vivo to an active embodiment of a compound. Typically, prodrugs are transformed in vivo to yield the embodiment of a compound, for example, by hydrolysis in blood or other organs by enzymatic processing. The prodrug compound often offers advantages of solubility, tissue compatibility or delayed release in the subject (see, Bundgard, H., Design of Prodrugs (1985), pp. 7-9, 21-24 (Elsevier, Amsterdam). The definition of prodrug includes any covalently bonded carriers which release the active compound in vivo when such prodrug is administered to a subject. Prodrugs of an embodiment of a compound may be prepared by modifying functional groups present in the compound in such a way that the modifications are cleaved, either in routine manipulation or in vivo, to a parent compound.

As used herein, the term “pharmaceutically acceptable carrier, diluent or excipient” is intended to mean, without limitation, any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, emulsifier, or encapsulating agent, such as a liposome, cyclodextrins, encapsulating polymeric delivery systems or polyethyleneglycol matrix, which is acceptable for use in the subject, preferably humans.

As used herein, the term “pharmaceutically acceptable salt” is intended to mean both acid and base addition salts.

As used herein, the term “pharmaceutically acceptable acid addition salt” is intended to mean those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid and the like, and organic acids such as acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

As used herein, the term “pharmaceutically acceptable base addition salt” is intended to mean those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like.

As used herein, the term “BIR domain binding” is intended to mean the action of a compound of embodiments upon an IAP BIR domain, which blocks or diminishes the binding of IAPs to BIR binding proteins or is involved in displacing BIR binding proteins from an IAP. Examples of BIR binding proteins include, but are not limited to, caspases and mitochondrially derived BIR binding proteins such as Smac, Omi/WTR2A and the like.

As used herein, the term “insufficient apoptosis” is intended to mean a state wherein a disease is caused or continues because cells deleterious to the subject have not apoptosed. This includes, but is not limited to, cancer cells that survive in a subject without treatment, cancer cells that survive in a subject during or following anti-cancer treatment, or immune cells whose action is deleterious to the subject, and includes, neutrophils, monocytes and auto-reactive T-cells.

As used herein, the term “therapeutically effective amount” is intended to mean an amount of a compound of Formula I which, when administered to a subject is sufficient to effect treatment for a disease-state associated with insufficient apoptosis. The amount of the compound of Formula I will vary depending on the compound, the condition and its severity, and the age of the subject to be treated, but can be determined routinely by one of ordinary skill in the art having regard to his own knowledge and to this disclosure.

As used herein, the term “treating” or “treatment” is intended to mean treatment of a disease-state associated with insufficient apoptosis, as disclosed herein, in a subject, and includes: (i) preventing a disease or condition associated with insufficient apoptosis from occurring in a subject, in particular, when such mammal is predisposed to the disease or condition but has not yet been diagnosed as having it; (ii) inhibiting a disease or condition associated with insufficient apoptosis, i.e., arresting its development; or (iii) relieving a disease or condition associated with insufficient apoptosis, i.e., causing regression of the condition.

As used herein, the term “treating cancer” is intended to mean the administration of a pharmaceutical composition comprising one or more embodiments of a compound to a subject, preferably a human, which is afflicted with cancer to cause an alleviation of the cancer by killing, inhibiting the growth, or inhibiting the metastasis of the cancer cells.

As used herein, the term “preventing disease” is intended to mean, in the case of cancer, the post-surgical, post-chemotherapy or post-radiotherapy administration of an embodiment of a pharmaceutical composition to a subject, preferably a human, which was afflicted with cancer to prevent the regrowth of the cancer by killing, inhibiting the growth, or inhibiting the metastasis of any remaining cancer cells. Also included in this definition is the prevention of prosurvival conditions that lead to diseases such as asthma, MS and the like.

As used herein, the term “synergistic effect” is intended to mean that the effect achieved with the combination of embodiments of the compounds and either the chemotherapeutic agents or death receptor agonists of embodiments of one or more compounds is greater than the effect which is obtained with only one of the compounds, agents or agonists, or advantageously the effect which is obtained with the combination of the above compounds, agents or agonists is greater than the addition of the effects obtained with each of the compounds, agents or agonists used separately. Such synergy enables smaller doses to be given.

As used herein, the term “apoptosis” or “programmed cell death” is intended to mean the regulated process of cell death wherein a dying cell displays a set of well-characterized biochemical hallmarks that include cell membrane blebbing, cell soma shrinkage, chromatin condensation, and DNA laddering, as well as any caspase-mediated cell death.

As used herein, the term “BIR domain” or “BIR” are used interchangeably throughout and are intended to mean a domain which is characterized by a number of invariant amino acid residue including conserved cysteines and one conserved hisitidine residue within the sequence Cys-(Xaa1)₂Cys-(Xaa1)₁₆His-(Xaa1)₆₋₈Cys. Typically, the amino acid sequence of the consensus sequence is: Xaa1-Xaa1-Xaa1-Arg-Leu-Xaa1-Thr-Phe-Xaa1-Xaa1-Trp-Pro-Xaa2-Xaa1-Xaa1-Xaa2-Xaa2-Xaa1-Xaa1-Xaa1-Xaa1-Leu-Ala-Xaa1-Ala-Gly-Phe-Tyr-Tyr-Xaa1-Gly-Xaa1-Xaa1-Asp-Xaa1-Val-Xaa1-Cys-Phe-Xaa1-Cys-Xaa1-Xaa1-Xaa1-Xaa1-Xaa1-Xaa1-Trp-Xaa1-Xaa1-Xaa1-Asp-Xaa1-Xaa1-Xaa1-Xaa1-Xaa1-His-Xaa-1-Xaa1-Xaa1-Xaa1-Pro-Xaa1-Cys-Xaa1-Phe-Val, wherein Xaa1 is any amino acid and Xaa2 is any amino acid or is absent. Preferably the sequence is substantially identical to one of the BIR domain sequences provided for XIAP, HIAP1, or HIAP2 herein.

The BIR domain residues are listed below (see Genome Biology (2001) 1-10):

| | XIAP | HIAP-1 | HIAP-2 |
|---|---|---|---|
| BIR1 | 21-93 | 41-113 | 24-96 |
| BIR2 | 159-230 | 179-250 | 164-235 |
| BIR3 | 258-330 | 264-336 | 250-322 |
| Seq. # | P98170 | XP-006266 | XP-006267 |

As used herein, the term “ring zinc finger” or “RZF” is intended to mean a domain having the amino acid sequence of the consensus sequence: Glu-Xaa1-Xaa1-Xaa1-Xaa1-Xaa1-Xaa-1-Xaa2-Xaa1-Xaa1-Xaa1-Cys-Lys-Xaa3-Cys-Met-Xaa1-Xaa1-Xaa1-Xaa1-Xaa1-Xaa3-X-aa1-Phe-Xaa1-Pro-Cys-Gly-His-Xaa1-Xaa1-Xaa1-Cys-Xaa1-Xaa1-Cys-Ala-Xaa1-Xaa-1-Xaa1-Xaa1-Xaa1-Cys-Pro-Xaa1-Cys, wherein Xaa1 is any amino acid, Xaa2 is Glu or Asp, and Xaa3 is Val or Ile.

As used herein, the term “TAP” is intended to mean a polypeptide or protein, or fragment thereof, encoded by an TAP gene. Examples of IAPs include, but are not limited to human or mouse NAIP (Birc 1), HIAP-1 (cIAP2, Birc 3), HIAP-2 (cIAP1, Birc 2), XIAP (Birc 4), survivin (Birc 5), livin (ML-IAP, Birc 7), ILP-2 (Birc 8) and Apollon/BRUCE (Birc 6) (see for example U.S. Pat. Nos. 6,107,041; 6,133,437; 6,156,535; 6,541,457; 6,656,704; 6,689,562; Deveraux and Reed, Genes Dev. 13, 239-252, 1999; Kasof and Gomes, J. Biol. Chem., 276, 3238-3246, 2001; Vucic et al., Curr. Biol. 10, 1359-1366, 2000; Ashab et al. FEBS Lett., 495, 56-60, 2001, the contents of which are hereby incorporated by reference).

As used herein, the term “TAP gene” is intended to mean a gene encoding a polypeptide having at least one BIR domain and which is capable of modulating (inhibiting or enhancing) apoptosis in a cell or tissue. The TAP gene is a gene having about 50% or greater nucleotide sequence identity to at least one of human or mouse NAIP (Birc 1), HIAP-1 (cIAP2, Birc 3), HIAP-2 (cIAP1, Birc 2), XIAP (Birc 4), survivin (Birc 5), livin (ML-IAP, Birc 7), ILP-2 (Birc 8) and Apollon/BRUCE (Birc 6). The region of sequence over which identity is measured is a region encoding at least one BIR domain and a ring zinc finger domain. Mammalian TAP genes include nucleotide sequences isolated from any mammalian source.

As used herein, the term “IC₅₀” is intended to mean an amount, concentration or dosage of a particular compound of embodiments that achieves a 50% inhibition of a maximal response, such as displacement of maximal fluorescent probe binding in an assay that measures such response.

As used herein, the term “EC₅₀” is intended to mean an amount, concentration or dosage of a particular compound of embodiments that achieves a 50% inhibition of cell survival.

As used herein, the term “modulate” or “modulating” is intended to mean the treatment, prevention, suppression, enhancement or induction of a function or condition using the compounds of some embodiments. For example, the compounds of some embodiments can modulate TAP function in a subject, thereby enhancing apoptosis by significantly reducing, or essentially eliminating the interaction of activated apoptotic proteins, such as caspase-3, 7 and 9, with the BIR domains of mammalian IAPs or by inducing the loss of IAP protein in a cell.

As used herein, the term “enhancing apoptosis” is intended to mean increasing the number of cells that apoptose in a given cell population either in vitro or in vivo. Examples of cell populations include, but are not limited to, ovarian cancer cells, colon cancer cells, breast cancer cells, lung cancer cells, pancreatic cancer cells, or T cells and the like. It will be appreciated that the degree of apoptosis enhancement provided by an apoptosis-enhancing embodiment of a compound in a given assay will vary, but that one skilled in the art can determine the statistically significant change in the level of apoptosis that identifies a compound that enhances apoptosis otherwise limited by an TAP. Preferably “enhancing apoptosis” means that the increase in the number of cells undergoing apoptosis is at least 25%, more preferably the increase is 50%, and most preferably the increase is at least one-fold. Preferably the sample monitored is a sample of cells that normally undergo insufficient apoptosis (i.e., cancer cells). Methods for detecting the changes in the level of apoptosis (i.e., enhancement or reduction) are described in the Examples and include methods that quantitate the fragmentation of DNA, methods that quantitate the translocation phosphatoylserine from the cytoplasmic to the extracellular side of the membrane, determination of activation of the caspases and methods quantitate the release of cytochrome C and the apoptosis inhibitory factor into the cytoplasm by mitochondria.

As used herein, the term “proliferative disease” or “proliferative disorder” is intended to mean a disease that is caused by or results in inappropriately high levels of cell division, inappropriately low levels of apoptosis, or both. For example, cancers such as lymphoma, leukemia, melanoma, ovarian cancer, breast cancer, pancreatic cancer, and lung cancer, and autoimmune disorders are all examples of proliferative diseases.

As used herein, the term “death receptor agonist” is intended to mean an agent capable of stimulating by direct or indirect contact the pro apoptotic response mediated by the death-receptors. For example, an agonist TRAIL receptor Antibody would bind to TRAIL receptor (S) and trigger an apoptotic response. On the other hand, other agent such as interferon-a could trigger the release of endogenous TRAIL and/or up regulate the TRAIL receptors in such a way that the cell pro-apoptotic response is amplified.

The compounds of embodiments of the present invention, or their pharmaceutically acceptable salts may contain one or more asymmetric centers, chiral axes and chiral planes and may thus give rise to enantiomers, diastereomers, and other stereoisomeric forms and may be defined in terms of absolute stereochemistry, such as (R)- or (S)- or, as (D)- or (L)- for amino acids. Embodiments of the present invention are intended to include all such possible isomers, as well as, their racemic and optically pure forms. Optically active (+) and (−), (R)- and (S)-, or (D)- and (L)-isomers may be prepared using chiral synthons or chiral reagents, or resolved using conventional techniques, such as reverse phase HPLC. The racemic mixtures may be prepared and thereafter separated into individual optical isomers or these optical isomers may be prepared by chiral synthesis. The enantiomers may be resolved by methods known to those skilled in the art, for example by formation of diastereoisomeric salts which may then be separated by crystallization, gas-liquid or liquid chromatography, selective reaction of one enantiomer with an enantiomer specific reagent. It will also be appreciated by those skilled in the art that where the desired enantiomer is converted into another chemical entity by a separation technique, an additional step is then required to form the desired enantiomeric form. Alternatively specific enantiomers may be synthesized by asymmetric synthesis using optically active reagents, substrates, catalysts, or solvents or by converting one enantiomer to another by asymmetric transformation.

Certain compounds of several embodiments may exist in Zwitterionic form and several embodiments include Zwitterionic forms of these compounds and mixtures thereof.

### Utilities

The compounds, according to several embodiments, are useful as IAP BIR domain binding compounds and as such the compounds, compositions and method include application to the cells or subjects afflicted with or having a predisposition towards developing a particular disease state, which is characterized by insufficient apoptosis. Thus, the compounds, compositions and methods, according to several embodiments, are used to treat cellular proliferative diseases/disorders, which include, but are not limited to, i) cancer, ii) autoimmune disease, iii) inflammatory disorders, iv) proliferation induced post medical procedures, including, but not limited to, surgery, angioplasty, and the like.

The compounds, according to several embodiments, may also be useful in the treatment of diseases in which there is a defect in the programmed cell-death or the apoptotic machinery (TRAIL, FAS, apoptosome), such as multiple sclerosis, asthma, arthrosclerosis, atherosclerosis, inflammation, autoimmunity and the like.

The treatment involves administration to a subject in need thereof a compound of several embodiments or a pharmaceutically acceptable salt thereof, or a pharmaceutical composition comprising a pharmaceutical carrier and a therapeutically effective amount of a compound of several embodiments, or a pharmaceutically acceptable salt thereof, or combinations thereof.

In particular, the compounds, compositions and methods, according to several embodiments, are useful for the treatment of cancer including solid tumors such as skin, breast, brain, lung, testicular carcinomas, and the like. Cancers that may be treated by the compounds, compositions and methods include, but are not limited to the following:

| Tissue | Example |
|---|---|
| Adrenal gland | neuroblastoma |
| Bone | osteogenic sarcoma (osteosarcoma), fibrosarcoma, malignant |
| | fibrous histiocytoma, chondrosarcoma, Ewing's sarcoma, |
| | malignant lymphoma (reticulum cell sarcoma), multiple |
| | myeloma, malignant giant cell tumor chordoma, |
| | osteochronfroma (osteocartilaginous exostoses), benign |
| | chondroma, chondroblastoma, chondromyxofibroma, osteoid |
| | osteoma and giant cell tumors |
| Cardiac | sarcoma (angiosarcoma, fibrosarcoma, rhabdomyosarcoma, |
| | liposarcoma), myxoma, rhabdomyoma, fibroma, lipoma and |
| | teratoma |
| Gastrointestinal | esophagus (squamous cell carcinoma, adenocarcinoma, |
| | leiomyosarcoma, lymphoma), stomach (carcinoma, lymphoma, |
| | leiomyosarcoma), pancreas (ductal adenocarcinoma, |
| | insulinoma, glucagonoma, gastrinoma, carcinoid tumors, |
| | vipoma), small bowel (adenocarcinoma, lymphoma, carcinoid |
| | tumors, Karposi's sarcoma, leiomyoma, hemangioma, lipoma, |
| | neurofibroma, fibroma), large bowel (adenocarcinoma, tubular |
| | adenoma, villous adenoma, hamartoma, leiomyoma) |
| Genitourinary | kidney (adenocarcinoma, Wilm's tumor [nephroblastoma], |
| tract | lymphoma, leukemia), bladder and urethra (squamous cell |
| | carcinoma, transitional cell carcinoma, adenocarcinoma), |
| | prostate (adenocarcinoma, sarcoma), testis (seminoma, |
| | teratoma, embryonal carcinoma, teratocarcinoma, |
| | choriocarcinoma, sarcoma, interstitial cell carcinoma, fibroma, |
| | fibroadenoma, adenomatoid tumors, lipoma) |
| Gynecological | uterus (endometrial carcinoma), cervix (cervical carcinoma, |
| | pre-tumor cervical dysplasia), ovaries (ovarian carcinoma |
| | [serous cystadenocarcinoma, mucinous cystadenocarcinoma, |
| | unclassified carcinoma], granulosa-thecal cell tumors, Sertoli- |
| | Leydig cell tumors, dysgerminoma, malignant teratoma), vulva |
| | (squamous cell carcinoma, intraepithelial carcinoma, |
| | adenocarcinoma, fibrosarcoma, melanoma), vagina (clear cell |
| | carcinoma, squamous cell carcinoma, botryoid sarcoma |
| | (embryonal rhabdomyosarcoma), fallopian tubes (carcinoma) |
| Hematologic | blood (myeloid leukemia [acute and chronic], acute |
| | lymphoblastic leukemia, chronic lymphocytic leukemia, |
| | myeloproliferative diseases, multiple myeloma, myelodysplastic |
| | syndrome), Hodgkin's disease, non-Hodgkin's lymphoma |
| | [malignant lymphoma] |
| Liver | hepatoma (hepatocellular carcinoma), cholangiocarcinoma, |
| | hepatoblastoma, angiosarcoma, hepatocellular adenoma, |
| | hemangioma |
| Lung | bronchogenic carcinoma (squamous cell, undifferentiated small |
| | cell, undifferentiated large cell, adenocarcinoma), alveolar |
| | (bronchiolar) carcinoma, bronchial adenoma, sarcoma, |
| | lymphoma, chondromatous hamartoma, mesothelioma |
| Nervous system | skull (osteoma, hemangioma, granuloma, xanthoma, osteitis |
| | deformans), meninges (meningioma, meningiosarcoma, |
| | gliomatosis), brain (astrocytoma, medulloblastoma, glioma, |
| | ependymoma, germinoma [pinealoma], glioblastoma multiform, |
| | oligodendroglioma, schwannoma, retinoblastoma, congenital |
| | tumors), spinal cord neurofibroma, meningioma, glioma, |
| | sarcoma) |
| Skin | malignant melanoma, basal cell carcinoma, squamous cell |
| | carcinoma, Karposi's sarcoma, moles dysplastic nevi, lipoma, |
| | angioma, dermatofibroma, keloids |

The compounds, according to several embodiments, or their pharmaceutically acceptable salts or their prodrugs, may be administered in pure form or in an appropriate pharmaceutical composition, and can be carried out via any of the accepted modes of Galenic pharmaceutical practice.

The pharmaceutical compositions, according to several embodiments, can be prepared by admixing a compound of embodiments with an appropriate pharmaceutically acceptable carrier, diluent or excipient, and may be formulated into preparations in solid, semi-solid, liquid or gaseous forms, such as tablets, capsules, powders, granules, ointments, solutions, suppositories, injections, inhalants, gels, microspheres, and aerosols. Typical routes of administering such pharmaceutical compositions include, without limitation, oral, topical, transdermal, inhalation, parenteral (subcutaneous injections, intravenous, intramuscular, intrasternal injection or infusion techniques), sublingual, ocular, rectal, vaginal, and intranasal. Pharmaceutical compositions, according to several embodiments, are formulated so as to allow the active ingredients contained therein to be bioavailable upon administration of the composition to a subject. Compositions that will be administered to a subject or patient take the form of one or more dosage units, where for example, a tablet may be a single dosage unit, and a container of one or more embodiments in aerosol form may hold a plurality of dosage units. Actual methods of preparing such dosage forms are known, or will be apparent, to those skilled in this art; for example, see Remington's Pharmaceutical Sciences, 18th Ed., (Mack Publishing Company, Easton, Pa., 1990). The composition to be administered will, in any event, contain a therapeutically effective amount of one or more embodiments of a compound, or a pharmaceutically acceptable salt thereof, for treatment of a disease-state as described above.

A pharmaceutical composition, according to several embodiments, may be in the form of a solid or liquid. In one aspect, the carrier(s) are particulate, so that the compositions are, for example, in tablet or powder form. The carrier(s) may be liquid, with the compositions being, for example, an oral syrup, injectable liquid or an aerosol, which is useful in, for example inhalatory administration.

For oral administration, the pharmaceutical composition is preferably in either solid or liquid form, where semi-solid, semi-liquid, suspension and gel forms are included within the forms considered herein as either solid or liquid.

As a solid composition for oral administration, the pharmaceutical composition may be formulated into a powder, granule, compressed tablet, pill, capsule, chewing gum, wafer or the like form. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent.

When the pharmaceutical composition is in the form of a capsule, e.g., a gelatin capsule, it may contain, in addition to materials of the above type, a liquid carrier such as polyethylene glycol or oil such as soybean or vegetable oil.

The pharmaceutical composition may be in the form of a liquid, e.g., an elixir, syrup, solution, emulsion or suspension. The liquid may be for oral administration or for delivery by injection, as two examples. When intended for oral administration, preferred composition contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

The liquid pharmaceutical compositions, according to several embodiments, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediamine tetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. An injectable pharmaceutical composition is preferably sterile.

A liquid pharmaceutical composition, according to several embodiments, used for either parenteral or oral administration should contain an amount of a compound such that a suitable dosage will be obtained. Typically, this amount is at least 0.01% of an embodiment of a compound in the composition. When intended for oral administration, this amount may be varied to be between 0.1 and about 70% of the weight of the composition. For parenteral usage, compositions and preparations according to embodiments are prepared so that a parenteral dosage unit contains between 0.01 to 1% by weight of an embodiment of the compound.

The pharmaceutical composition, according to several embodiments, may be used for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a pharmaceutical composition for topical administration. If intended for transdermal administration, the composition may include a transdermal patch or iontophoresis device. Topical formulations may contain a concentration of the compound, according to several embodiments, from about 0.1 to about 10% w/v (weight per unit volume).

The pharmaceutical composition, according to several embodiments, may be used for rectal administration to treat for example, colon cancer, in the form, e.g., of a suppository, which will melt in the rectum and release the drug. The composition for rectal administration may contain an oleaginous base as a suitable nonirritating excipient. Such bases include, without limitation, lanolin, cocoa butter and polyethylene glycol.

The pharmaceutical composition, according to several embodiments, may include various materials, which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule.

The pharmaceutical composition, according to several embodiments, in solid or liquid form may include an agent that binds to embodiments of the compound and thereby assists in the delivery of the compound. Suitable agents that may act in this capacity include, but are not limited to, a monoclonal or polyclonal antibody, a protein or a liposome.

The pharmaceutical composition, according to several embodiments, may comprise or consist of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols of embodiments of compounds may be delivered in single phase, bi-phasic, or tri-phasic systems in order to deliver the active ingredient(s). Delivery of the aerosol includes the necessary container, activators, valves, subcontainers, and the like, which together may form a kit. One skilled in the art, without undue experimentation may determine preferred aerosols.

The pharmaceutical compositions, according to several embodiments, may be prepared by methodology well known in the pharmaceutical art. For example, a pharmaceutical composition intended to be administered by injection can be prepared by admixing embodiments of a compound with sterile, distilled water so as to form a solution. A surfactant may be added to facilitate the formation of a homogeneous solution or suspension. Surfactants are compounds that non-covalently interact with embodiments of the compound so as to facilitate dissolution or homogeneous suspension of the compound in the aqueous delivery system.

The compounds, according to several embodiments, or their pharmaceutically acceptable salts, are administered in a therapeutically effective amount, which will vary depending upon a variety of factors including the activity of the specific compound employed; the metabolic stability and length of action of the compound; the age, body weight, general health, sex, and diet of the patient; the mode and time of administration; the rate of excretion; the drug combination; the severity of the particular disorder or condition; and the subject undergoing therapy. Generally, a therapeutically effective daily dose may be from about 0.1 mg to about 40 mg/kg of body weight per day or twice per day of embodiments of a compound, or a pharmaceutically acceptable salt thereof.

### Combination Therapy

The compounds, according to several embodiments, or pharmaceutically acceptable salts thereof, may also be administered simultaneously with, prior to, or after administration of one or more of the therapeutic agents described below. Such combination therapy may include administration of a single pharmaceutical dosage formulation which contains embodiments of a compound and one or more additional agents given below, as well as administration of the compound and each additional agent in its own separate pharmaceutical dosage formulation. For example, embodiments of a compound and a chemotherapeutic agent, such as taxol (paclitaxel), taxotere, etoposide, cisplatin, vincristine, vinblastine, and the like, can be administered to the patient either together in a single dosage composition, or each agent administered in separate oral dosage formulations or via intravenous injection. Where separate dosage formulations are used, the compounds of some embodiments and one or more additional agents can be administered at essentially the same time, i.e., concurrently, or at separately staggered times, i.e., sequentially; combination therapy is understood to include all these regimens. In addition, these compounds may synergize with molecules that may stimulate the death receptor apoptotic pathway through a direct or indirect manner, as for example, the compounds of some embodiments may be used in combination with soluble TRAIL or any agent that can cause an increase in circulating level of TRAIL, such as interferon-alpha, BCG, or though radiation.

Thus, according to several embodiments, also encompasses the use of the compounds of some embodiments in combination with radiation therapy or one or more additional agents such as those described in WO 03/099211 (PCT/US03/15861), which is hereby incorporated by reference.

Examples of such additional agents include, but are not limited to the following:

a) an estrogen receptor modulator,
b) an androgen receptor modulator,
c) retinoid receptor modulator,
d) a cytotoxic agent,
e) an antiproliferative agent,
f) a prenyl-protein transferase inhibitor,
g) an HMG-CoA reductase inhibitor,
h) an HIV protease inhibitor,
i) a reverse transcriptase inhibitor,
k) an angiogenesis inhibitor,
l) a PPAR-.γ agonist,
m) a PPAR-δ. agonist,
n) an inhibitor of inherent multidrug resistance,
o) an anti-emetic agent,
p) an agent useful in the treatment of anemia,
q) agents useful in the treatment of neutropenia,
r) an immunologic-enhancing drug.
s) a proteasome inhibitor such as Velcade and MG132 (7-Leu-Leu-aldehyde) (see He at al. in Oncogene (2004) 23, 2554-2558);
t) an HDAC inhibitor, such as sodium butyrate, phenyl butyrate, hydroamic acids, cyclin tetrapeptide and the like (see Rosato et al., Molecular Cancer Therapeutics 2003, 1273-1284);
u) an inhibitor of the chemotrypsin-like activity in the proteasome;
v) E3 ligase inhibitors;
w) a modulator of the immune system such as, but not limited to, interferon-alpha or BCG that can induce the release of cytokines, such as the interleukins, TNF, or induce release of death receptor ligands such as TRAIL;
x) a modulator of death receptors TRAIL and TRAIL agonists such as the humanized antibodies HGS-ETR1 and HGS-ETR2; and
or in combination or sequentially with radiation therapy, so as to treat the cancer.

Additional combinations may also include agents which reduce the toxicity of the aforesaid agents, such as hepatic toxicity, neuronal toxicity, nephrotoxicity and the like.

In one example, co-administration of one of the compounds of Formula I, according to several embodiments, with a death receptor agonist such as TRAIL, such as a small molecule or an antibody that mimics TRAIL may cause an advantageous synergistic effect. Moreover, according to several embodiments, some compounds may be used in combination with any compounds that cause an increase in circulating levels of TRAIL.

### Vinca Alkaloids and Related Compounds

Vinca alkaloids that can be used in combination with embodiments of the nucleobase oligomers to treat cancer and other neoplasms include vincristine, vinblastine, vindesine, vinflunine, vinorelbine, and anhydrovinblastine.

Dolastatins are oligopeptides that primarily interfere with tubulin at the vinca alkaloid binding domain. These compounds can also be used in combination with embodiments of the compounds to treat cancer and other neoplasms. Dolastatins include dolastatin-10 (NCS 376128), dolastatin-15, ILX651, TZT-1027, symplostatin 1, symplostatin 3, and LU103793 (cemadotin). Cryptophycins (e.g., cryptophycin 1 and cryptophycin 52 (LY355703)) bind tubulin within the vinca alkaloid-binding domain and induce G2/M arrest and apoptosis. Any of these compounds can be used in combination with embodiments of the compounds to treat cancer and other neoplasms.

Other microtubule disrupting compounds that can be used in conjunction with embodiments of the compounds to treat cancer and other neoplasms are described in U.S. Pat. Nos. 6,458,765; 6,433,187; 6,323,315; 6,258,841; 6,143,721; 6,127,377; 6,103,698; 6,023,626; 5,985,837; 5,965,537; 5,955,423; 5,952,298; 5,939,527; 5,886,025; 5,831,002; 5,741,892; 5,665,860; 5,654,399; 5,635,483; 5,599,902; 5,530,097; 5,521,284; 5,504,191; 4,879,278; and 4,816,444, and U.S. patent application Publication Nos. 2003/0153505 A1; 2003/0083263 A1; and 2003/0055002 A1, each of which is hereby incorporated by reference.

### Taxanes and Other Micortubule Stabilizing Compounds

Taxanes such as paclitaxel, doxetaxel, RPR 109881A, SB-T-1213, SB-T-1250, SB-T-101187, BMS-275183, BRT 216, DJ-927, MAC-321, IDN5109, and IDN5390 can be used in combination with embodiments of the compounds to treat cancer and other neoplasms. Taxane analogs (e.g., BMS-184476, BMS-188797) and functionally related non-taxanes (e.g., epothilones (e.g., epothilone A, epothilone B (EP0906), deoxyepothilone B, and epothilone B lactam (BMS-247550)), eleutherobin, discodermolide, 2-epi-discodermolide, 2-des-methyldiscodermolide, 5-hydroxymethyldiscoder-molide, 19-des-aminocarbonyldiscodermolide, 9(13)-cyclodiscodermolide, and laulimalide) can also be used in embodiments of the methods and compositions.

Other microtubule stabilizing compounds that can be used in combination with embodiments of the compounds to treat cancer and other neoplasms are described in U.S. Pat. Nos. 6,624,317; 6,610,736; 6,605,599; 6,589,968; 6,583,290; 6,576,658; 6,515,017; 6,531,497; 6,500,858; 6,498,257; 6,495,594; 6,489,314; 6,458,976; 6,441,186; 6,441,025; 6,414,015; 6,387,927; 6,380,395; 6,380,394; 6,362,217; 6,359,140; 6,306,893; 6,302,838; 6,300,355; 6,291,690; 6,291,684; 6,268,381; 6,262,107; 6,262,094; 6,147,234; 6,136,808; 6,127,406; 6,100,411; 6,096,909; 6,025,385; 6,011,056; 5,965,718; 5,955,489; 5,919,815; 5,912,263; 5,840,750; 5,821,263; 5,767,297; 5,728,725; 5,721,268; 5,719,177; 5,714,513; 5,587,489; 5,473,057; 5,407,674; 5,250,722; 5,010,099; and 4,939,168; and U.S. patent application Publication Nos. 2003/0186965 A1; 2003/0176710 A1; 2003/0176473 A1; 2003/0144523 A1; 2003/0134883 A1; 2003/0087888 A1; 2003/0060623 A1; 2003/0045711 A1; 2003/0023082 A1; 2002/0198256 A1; 2002/0193361 A1; 2002/0188014 A1; 2002/0165257 A1; 2002/0156110 A1; 2002/0128471 A1; 2002/0045609 A1; 2002/0022651 A1; 2002/0016356 A1; 2002/0002292 A1, each of which is hereby incorporated by reference.

Other chemotherapeutic agents that may be administered with embodiments of a compound, according to several embodiments, are listed in the following Table:

| | | |
|---|---|---|
| Alkylating | cyclophosphamide | mechlorethamine |
| agents | lomustine | thiotepa |
| | busulfan | streptozocin |
| | procarbazine | chlorambucil |
| | ifosfamide | temozolomide |
| | altretamine | dacarbazine |
| | melphalan | semustine |
| | estramustine phosphate | carmustine |
| | hexamethylmelamine | |
| Platinum agents | cisplatin | tetraplatin |
| | carboplatinum | BBR-3464 (Hoffmann-La Roche) |
| | oxaliplatin | Ormiplatin |
| | ZD-0473 (AnorMED) | SM-11355 (Sumitomo) |
| | spiroplatinum | iproplatin |
| | lobaplatin (Aeterna) | AP-5280 (Access) |
| | carboxyphthalatoplatinum | |
| | satraplatin (Johnson Matthey) | |
| Antimetabolites | azacytidine | 6-mercaptopurine |
| | tomudex | hydroxyurea |
| | gemcitabine | 6-thioguanine |
| | trimetrexate | decitabine (SuperGen) |
| | capecitabine | cytarabin |
| | deoxycoformycin | clofarabine (Bioenvision) |
| | 5-fluorouracil | 2-fluorodeoxy |
| | fludarabine | cytidine |
| | floxuridine | irofulven (MGI Pharma) methotrexate |
| | pentostatin | DMDC (Hoffmann-La Roche) |
| | 2-chlorodeoxyadenosine | idatrexate |
| | raltitrexed | ethynylcytidine (Taiho) |
| Topoisomerase | amsacrine | TAS-103 (Taiho) |
| inhibitors | rubitecan (SuperGen) | Topotecan |
| | epirubicin | elsamitrucin (Spectrum) dexrazoxanet |
| | exatecan mesylate (Daiichi) | (TopoTarget) |
| | etoposide | J-107088 (Merck & Co) |
| | quinamed (ChemGenex) | pixantrone (Novuspharma) |
| | teniposide or mitoxantrone | BNP-1350 (BioNumerik) |
| | gimatecan (Sigma-Tau) | rebeccamycin analogue (Exelixis) |
| | irinotecan (CPT-11) | CKD-602 (Chong Kun Dang) |
| | diflomotecan (Beaufour-Ipsen) | BBR-3576 (Novuspharma) |
| | 7-ethyl-10-hydroxy-camptothecin | KW-2170 (Kyowa Hakko) |
| Antitumor | dactinomycin (actinomycin D) | bleomycinic acid |
| antibiotics | amonafide | idarubicin |
| | doxorubicin (adriamycin) | bleomycin A |
| | azonafide | rubidazone |
| | deoxyrubicin | bleomycin B |
| | anthrapyrazole | plicamycinp |
| | valrubicin | mitomycin C |
| | oxantrazole | porfiromycin |
| | daunorubicin (daunomycin) | MEN-10755 (Menarini) |
| | losoxantrone | cyanomorpholinodoxorubicin |
| | epirubicin | GPX-100 (Gem Pharmaceuticals) |
| | bleomycin sulfate (blenoxane) | mitoxantrone (novantrone) |
| | therarubicin | |
| Antimitotic | paclitaxel | RPR 109881A (Aventis) |
| agents | SB 408075 (GlaxoSmithKline) | ZD 6126 (AstraZeneca) |
| | docetaxel | TXD 258 (Aventis) |
| | E7010 (Abbott) | PEG-paclitaxel (Enzon) |
| | Colchicines | epothilone B (Novartis) |
| | PG-TXL (Cell Therapeutics) | AZ10992 (Asahi) |
| | vinblastine | T 900607 (Tularik) |
| | IDN 5109 (Bayer) | IDN-5109 (Indena) |
| | Vincristine | T 138067 (Tularik) |
| | A 105972 (Abbott) | AVLB (Prescient NeuroPharma) |
| | Vinorelbine | cryptophycin 52 (Eli Lilly) |
| | A 204197 (Abbott) | azaepothilone B (BMS) |
| | Vindesine | vinflunine (Fabre) |
| | LU 223651 (BASF) | BNP-7787 (BioNumerik) |
| | dolastatin 10 (NCI) | auristatin PE (Teikoku Hormone) |
| | D 24851 (ASTAMedica) | CA-4 prodrug (OXiGENE) |
| | rhizoxin (Fujisawa) | BMS 247550 (BMS) |
| | ER-86526 (Eisai) | dolastatin-10 (NIH) |
| | mivobulin (Warner-Lambert) | BMS 184476(BMS) |
| | combretastatin A4 (BMS) | CA-4 (OXiGENE) |
| | cemadotin (BASF) | BMS 188797 (BMS) |
| | isohomohalichondrin-B (PharmaMar) | taxoprexin (Protarga) |
| Aromatase | Aminoglutethimide | anastrazole |
| inhibitors | Exemestane | YM-511 (Yamanouchi) |
| | Letrozole | formestane |
| | atamestane (BioMedicines) | |
| Thymidylate | pemetrexed (Eli Lilly) | ZD-9331 (BTG) |
| synthase | nolatrexed (Eximias) | CoFactor ™ (BioKeys) |
| inhibitors | | |
| DNA | trabectedin (PharmaMar) | albumin + 32P (Isotope Solutions) |
| antagonists | mafosfamide (Baxter International) | O6 benzyl guanine (Paligent) |
| | glufosfamide (Baxter International) | thymectacin (NewBiotics) |
| | apaziquone (Spectrum | edotreotide (Novartis) |
| | Pharmaceuticals) | |
| Farnesyltransferase | arglabin (NuOncology Labs) | perillyl alcohol (DOR BioPharma) |
| inhibitors | tipifarnib (Johnson & Johnson) | BAY-43-9006 (Bayer) |
| | lonafarnib (Schering-Plough) | |
| Pump inhibitors | CBT-1 (CBA Pharma) | tariquidar (Xenova) |
| | zosuquidar trihydrochloride (Eli | biricodar dicitrate (Vertex) |
| | Lilly) | MS-209 (Schering AG) |
| Histone | tacedinaline (Pfizer) | depsipeptide (Fujisawa) |
| acetyltransferase | pivaloyloxymethyl butyrate (Titan) | MS-275 (Schering AG) |
| inhibitors | SAHA (Aton Pharma) | |
| Metalloproteinase | Neovastat (Aeterna Laboratories) | marimastat (British Biotech) |
| inhibitors | CMT-3 (CollaGenex) | BMS-275291 (Celltech) |
| Ribonucleoside | gallium maltolate (Titan) | triapine (Vion) |
| reductase | tezacitabine (Aventis) | didox (Molecules for Health) |
| inhibitors | | |
| TNF alpha | virulizin (Lorus Therapeutics) | CDC-394 (Celgene) |
| agonists/ | revimid (Celgene) | |
| antagonists | | |
| Endothelin A | atrasentan (Abbott) | ZD-4054 (AstraZeneca) |
| receptor | YM-598 (Yamanouchi) | |
| antagonist | | |
| Retinoic acid | fenretinide (Johnson & Johnson) | LGD-1550 (Ligand) |
| receptor agonists | alitretinoin (Ligand) | |
| Immuno- | Interferon | norelin (Biostar) |
| modulators | dexosome therapy (Anosys) | IRX-2 (Immuno-Rx) |
| | oncophage (Antigenics) | BLP-25 (Biomira) |
| | pentrix (Australian Cancer | PEP-005 (Peplin Biotech) |
| | Technology) | MGV (Progenics) |
| | GMK (Progenics) | synchrovax vaccines (CTL Imimmo) |
| | ISF-154 (Tragen) | beta.-alethine (Dovetail) |
| | adenocarcinoma vaccine (Biomira) | melanoma vaccine (CTL Immuno) |
| | cancer vaccine (Intercell) | CLL therapy (Vasogen) |
| | CTP-37 (A VI BioPharma) | p21 RAS vaccine (GemVax) |
| Hormonal and | estrogens | bicalutamide |
| antihormonal | Prednisone | testosterone propionate; |
| agents | conjugated estrogens | fluoxymesterone |
| | methylprednisolone | flutamide |
| | ethinyl estradiol | methyltestosterone |
| | prednisolone | octreotide |
| | chlortrianisen | diethylstilbestrol |
| | aminoglutethimide | nilutamide |
| | idenestrol | megestrol |
| | leuprolide | mitotane tamoxifen |
| | hydroxyprogesterone caproate | P-04 (Novogen) |
| | goserelin | Toremofine |
| | medroxyprogesterone | 2-methoxyestradiol (EntreMed) |
| | leuporelin | dexamethasone |
| | testosterone | arzoxifene (Eli Lilly) |
| Photodynamic | talaporfin (Light Sciences) | motexafin |
| agents | Pd-bacteriopheophorbide (Yeda) | gadolinium (Pharmacyclics) |
| | Theralux (Theratechnologies) | hypericin |
| | lutetium texaphyrin (Pharmacyclics) | |
| Tyrosine Kinase | imatinib (Novartis) | C225 (ImClone) |
| Inhibitors | kahalide F (PharmaMar) | ZD4190 (AstraZeneca) |
| | leflunomide (Sugen/Pharmacia) | rhu-Mab (Genentech) |
| | CEP-701 (Cephalon) | ZD6474 (AstraZeneca) |
| | ZD1839 (AstraZeneca) | MDX-H210 (Medarex) |
| | CEP-751 (Cephalon) | vatalanib (Novartis) |
| | erlotinib (Oncogene Science) | 2C4 (Genentech) |
| | MLN518 (Millenium) | PKI166 (Novartis) |
| | canertinib (Pfizer) | MDX-447 (Medarex) |
| | PKC412 (Novartis) | GW2016 (GlaxoSmithKline) |
| | squalamine (Genaera) | ABX-EGF (Abgenix) |
| | phenoxodiol ( ) | EKB-509 (Wyeth) |
| | SU5416 (Pharmacia) | IMC-1C11 (ImClone) |
| | trastuzumab (Genentech) | EKB-569 (Wyeth) |
| | SU6668 (Pharmacia) | |

Miscellaneous agents

| | |
|---|---|
| SR-27897 (CCK A inhibitor, Sanofi- | SR-31747 (IL-1 antagonist, Sanofi- |
| Synthelabo) | Synthelabo) |
| BCX-1777 (PNP inhibitor, BioCryst) | gemtuzumab (CD33 antibody, Wyeth |
| tocladesine (cyclic AMP agonist, | Ayerst) |
| Ribapharm) | CCI-779 (mTOR kinase inhibitor, Wyeth) |
| ranpirnase (ribonuclease stimulant, Alfacell) | PG2 (hematopoiesis enhancer, |
| alvocidib (CDK inhibitor, Aventis) | Pharmagenesis) |
| galarubicin (RNA synthesis inhibitor, | exisulind (PDE V inhibitor, Cell Pathways) |
| Dong-A) | Immunol ™ (triclosan oral rinse, Endo) |
| CV-247 (COX-2 inhibitor, Ivy Medical) | CP-461 (PDE V inhibitor, Cell Pathways) |
| tirapazamine (reducing agent, SRI | triacetyluridine (uridine prodrug, Wellstat) |
| International) | AG-2037 (GART inhibitor, Pfizer) |
| P54 (COX-2 inhibitor, Phytopharm) | SN-4071 (sarcoma agent, Signature |
| N-acetylcysteine (reducing agent, Zambon) | BioScience) WX-UK1 (plasminogen |
| CapCell ™ (CYP450 stimulant, Bavarian | activator inhibitor, Wilex) |
| Nordic) | TransMID-107. TM. (immunotoxin, KS |
| R-flurbiprofen (NF-kappaB inhibitor, | Biomedix) |
| Encore) | PBI-1402 (PMN stimulant, ProMetic |
| GCS-100 (gal3 antagonist, GlycoGenesys) | LifeSciences) |
| 3CPA (NF-kappaB inhibitor, Active | PCK-3145 (apoptosis promotor, Procyon) |
| Biotech) | bortezomib (proteasome inhibitor, |
| G17DT immunogen (gastrin inhibitor, | Millennium) |
| Aphton) | doranidazole (apoptosis promotor, Pola) |
| seocalcitol (vitamin D receptor agonist, Leo) | SRL-172 (T cell stimulant, SR Pharma) |
| efaproxiral (oxygenator, Allos Therapeutics) | CHS-828 (cytotoxic agent, Leo) |
| 131-I-TM-601 (DNA antagonist, | TLK-286 (glutathione S transferase |
| TransMolecular) | inhibitor, Telik) |
| PI-88 (heparanase inhibitor, Progen) | trans-retinoic acid (differentiator, NIH) |
| eflornithine (ODC inhibitor, ILEX | PT-100 (growth factor agonist, Point |
| Oncology) | Therapeutics) |
| tesmilifene (histamine antagonist, YM | MX6 (apoptosis promotor, MAXIA) |
| BioSciences) | midostaurin (PKC inhibitor, Novartis) |
| minodronic acid (osteoclast inhibitor, | apomine (apoptosis promotor, ILEX |
| Yamanouchi) | Oncology) |
| histamine (histamine H2 receptor agonist, | bryostatin-1 (PKC stimulant, GPC Biotech) |
| Maxim) | urocidin (apoptosis promotor, Bioniche) |
| indisulam (p53 stimulant, Eisai) | CDA-II (apoptosis promotor, Everlife) |
| tiazofurin (IMPDH inhibitor, Ribapharm) | Ro-31-7453 (apoptosis promotor, La |
| aplidine (PPT inhibitor, PharmaMar) | Roche) |
| cilengitide (integrin antagonist, Merck | SDX-101 (apoptosis promotor, Salmedix) |
| KGaA) | brostallicin (apoptosis promotor, Pharmacia) |
| rituximab (CD20 antibody, Genentech) | ceflatonin (apoptosis promotor, |
| | ChemGenex) |

Additional combinations may also include agents which reduce the toxicity of the aforesaid agents, such as hepatic toxicity, neuronal toxicity, nephprotoxicity and the like.

### Screening Assays

The compounds, according to several embodiments, may also be used in a method to screen for other compounds that bind to an IAP BIR domain. Generally speaking, to use the compounds in a method of identifying compounds that bind to an IAP BIR domain, the IAP is bound to a support, and a compound is added to the assay. Alternatively, the compound may be bound to the support and the IAP is added.

There are a number of ways in which to determine the binding of a compound, according to several embodiments, to the BIR domain. In one way, an embodiment of the compound, for example, may be fluorescently or radioactively labeled and binding determined directly. For example, this may be done by attaching the IAP to a solid support, adding a detectably labeled compound, washing off excess reagent, and determining whether the amount of the detectable label is that present on the solid support. Numerous blocking and washing steps may be used, which are known to those skilled in the art.

In some cases, only one of the components is labeled. For example, specific residues in the BIR domain may be labeled. Alternatively, more than one component may be labeled with different labels; for example, using I¹²⁵ for the BIR domain, and a fluorescent label for the probe.

The compounds may also be used as competitors to screen for additional drug candidates or test compounds. As used herein, the terms “drug candidate” or “test compounds” are used interchangeably and describe any molecule, for example, protein, oligopeptide, small organic molecule, polysaccharide, polynucleotide, and the like, to be tested for bioactivity. The compounds may be capable of directly or indirectly altering the IAP biological activity.

Drug candidates can include various chemical classes, although typically they are small organic molecules having a molecular weight of more than 100 and less than about 2,500 Daltons. Candidate agents typically include functional groups necessary for structural interaction with proteins, for example, hydrogen bonding and lipophilic binding, and typically include at least an amine, carbonyl, hydroxyl, ether, or carboxyl group. The drug candidates often include cyclical carbon or heterocyclic structures and/or aromatic or polyaromatic structures substituted with one or more functional groups.

Drug candidates can be obtained from any number of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of bacterial, fungal, plant and animal extracts are available or readily produced. Additionally, natural or synthetically produced libraries and compounds are readily modified through conventional chemical, physical and biochemical means.

Competitive screening assays may be done by combining an IAP BIR domain and a probe to form a probe: BIR domain complex in a first sample followed by adding a test compound from a second sample. The binding of the test is determined, and a change, or difference in binding between the two samples indicates the presence of a test compound capable of binding to the BIR domain and potentially modulating the IAP's activity.

In one case, the binding of the test compound is determined through the use of competitive binding assays. In this embodiment, the probe is labeled with an affinity label such as biotin. Under certain circumstances, there may be competitive binding between the test compound and the probe, with the probe displacing the candidate agent.

In one case, the test compound may be labeled. Either the test compound, or an embodiment of a compound, or both, is added first to the IAP BIR domain for a time sufficient to allow binding to form a complex.

Formation of the probe: BIR domain complex typically require Incubations of between 4° C. and 40° C. for between 10 minutes to about 1 hour to allow for high-throughput screening. Any excess of reagents are generally removed or washed away. The test compound is then added, and the presence or absence of the labeled component is followed, to indicate binding to the BIR domain.

In one case, the probe is added first, followed by the test compound. Displacement of the probe is an indication the test compound is binding to the BIR domain and thus is capable of binding to, and potentially modulating, the activity of IAP. Either component can be labeled. For example, the presence of probe in the wash solution indicates displacement by the test compound. Alternatively, if the test compound is labeled, the presence of the probe on the support indicates displacement.

In one case, the test compound may be added first, with incubation and washing, followed by the probe. The absence of binding by the probe may indicate the test compound is bound to the BIR domain with a higher affinity. Thus, if the probe is detected on the support, coupled with a lack of test compound binding, may indicate the test compound is capable of binding to the BIR domain.

Modulation is tested by screening for a test compound's ability to modulate the activity of IAP and includes combining a test compound with an IAP BIR domain, as described above, and determining an alteration in the biological activity of the IAP. Therefore in this case, the test compound should both bind to the BIR domain (although this may not be necessary), and alter its biological activity as defined herein.

Positive controls and negative controls may be used in the assays. All control and test samples are performed multiple times to obtain statistically significant results. Following incubation, all samples are washed free of non-specifically bound material and the amount of bound probe determined. For example, where a radiolabel is employed, the samples may be counted in a scintillation counter to determine the amount of bound compound.

Typically, the signals that are detected in the assay may include fluorescence, resonance energy transfer, time resolved fluorescence, radioactivity, fluorescence polarization, plasma resonance, or chemiluminescence and the like, depending on the nature of the label. Detectable labels useful in performing screening assays include a fluorescent label such as Fluorescein, Oregon green, dansyl, rhodamine, tetramethyl rhodamine, texas red, Eu³⁺; a chemiluminescent label such as luciferase; colorimetric labels; enzymatic markers; or radioisotopes such as tritium, I¹²⁵ and the like

Affinity tags, which may be useful in performing the screening assays of some embodiments include be biotin, polyhistidine and the like.

### Synthesis and Methodology

General methods for the synthesis of embodiments of the compounds are shown below and are disclosed merely for the purpose of illustration and are not meant to be interpreted as limiting the processes to make the compounds by any other methods. Those skilled in the art will readily appreciate that a number of methods are available for the preparation of embodiments of the compounds. A number of intermediate compounds disclosed herein may be synthesized using synthetic methods disclosed in previously filed United States provisional patent application (see Appendix A), the entire contents of which is hereby incorporated by reference.

Scheme 1 illustrates the synthesis of a typical synthetic intermediate represented by 1(i). Examples of 1(i) represent proline derivatives such as 1(ii) and 2-(aminomethyl)pyrrolidine derivatives represented by intermediates 1 (iii-viii). Proline derivatives of 1(i) may be prepared by the treatment of Boc-Pro-OH with typical peptide coupling agents and an amine, to provide intermediate 1(ii). The 2-(aminomethyl)pyrrolidine intermediate 1(iii) is prepared by the condensation of an amine with N-Boc-prolinal. The resulting amine may be acylated with an acid chloride, anhydride or suitably activated carboxylic acid, such as succinamidyl esters, HOBt esters and the like, to provide intermediates such as 1(iv-vi). The intermediates 1(iv) and 1(v) feature protecting groups, which may be further removed and functionalized later in the synthesis. Sulfonylation with a sulfonyl chloride provides 1(vii). Appropriately activated, side chain protected amino acids may be coupled to intermediate 1(iii) using standard peptide coupling agents to provide intermediate 1(viii), the PG can be removed later in the synthesis.

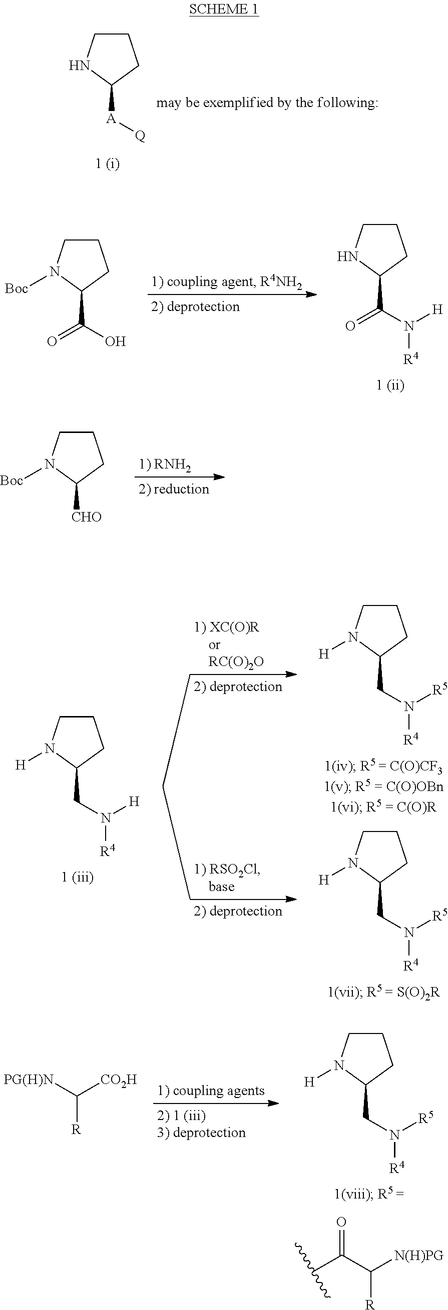

### General Procedure for the Preparation of Intermediate 2iii.

Scheme 2 illustrates a general procedure for preparing intermediate 2iii. PG¹-Thr-OH is deprotonated with NaH and treated with propargyl bromide to provide the Thr intermediate 2(i). Activation of the carboxylic acid of 2(i) with standard peptide coupling agents and treatment with intermediate 1(i), and deprotection at PG¹, provides the amide intermediate 2(ii). Peptide coupling of PG²(R¹)N(R²)(H)CCO₂H with 2(ii) is effected by activation of the carboxylic acid of PG²(R¹)N(R²)(H)CCO₂H with standard peptide coupling agents, followed by the addition of 2(ii) to provide the fully protected amide 2(iii).

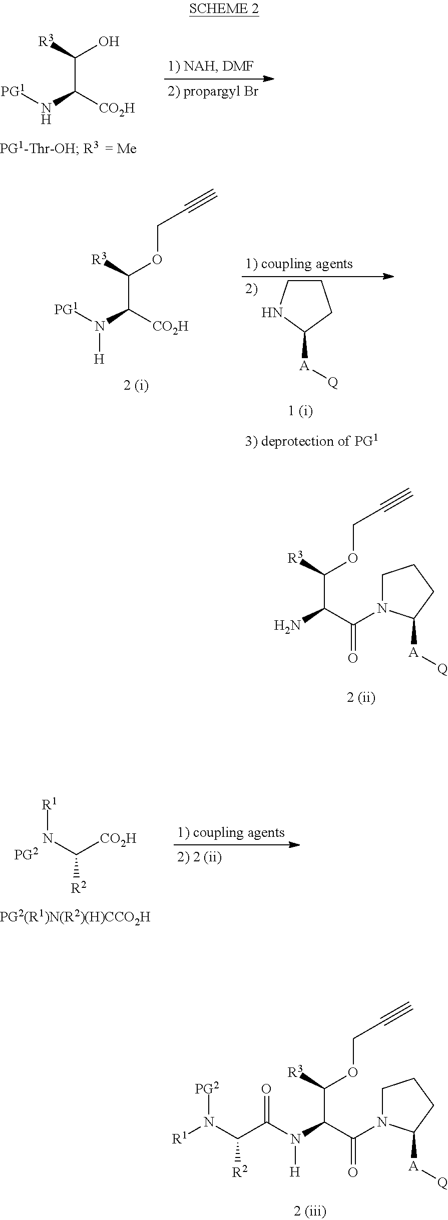

### General Procedure for the Preparation of Intermediate 3(ii).

Scheme 3 illustrates a general procedure for preparing intermediate 3(ii). Activation of the carboxylic acid of PG³-4-iodo-phenylalanine with standard peptide coupling agents and treatment with intermediate 1(i), and deprotection at PG³, provides the amide intermediate 3(i). Peptide coupling of PG⁴(R¹)N(R²)(H)CCO₂H with 3(i) is effected by activation of the carboxylic acid of PG⁴(R¹)N(R²)(H)CCO₂H with standard peptide coupling agents, followed by the addition of 3(i) to provide intermediate 3(ii).

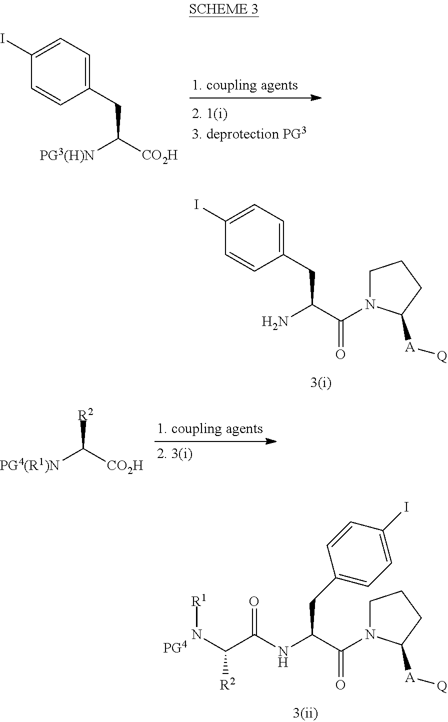

### General Procedure for the Preparation of Intermediate 4(i).

Scheme 4 illustrates a general procedure for preparing the boronic acid intermediate 4(i). Intermediate 3(ii) is treated with pinicol diborane and an appropriate Pd catalyst system to provide the pinicol borane intermediate 4(i). Intermediate 4(i) is converted to the boronic acid intermediate 4(ii) by treatment, for example, with NaOAc and NaIO₄.

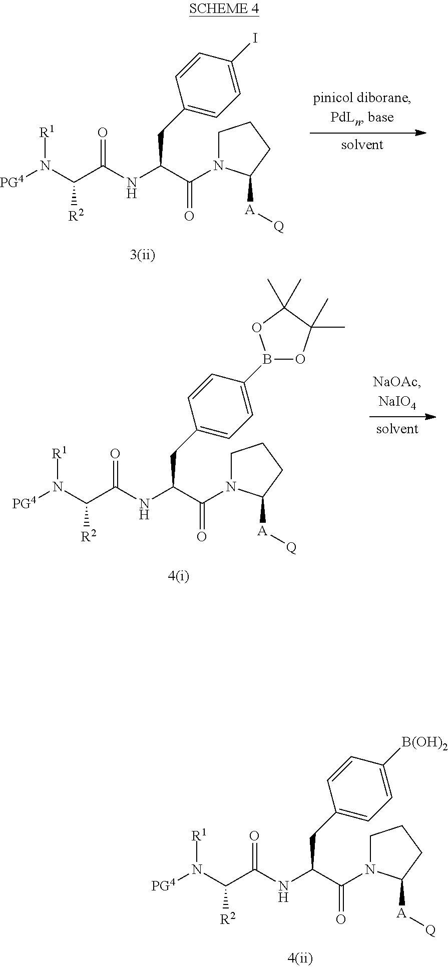

### General Procedure for the Preparation of Intermediate 5(i).

Scheme 4 illustrates a general procedure for preparing intermediate 5(i). Intermediate 3(ii) is treated with trimethylsilylacetylene and an appropriate Pd catalyst system to provide intermediate 5(i).

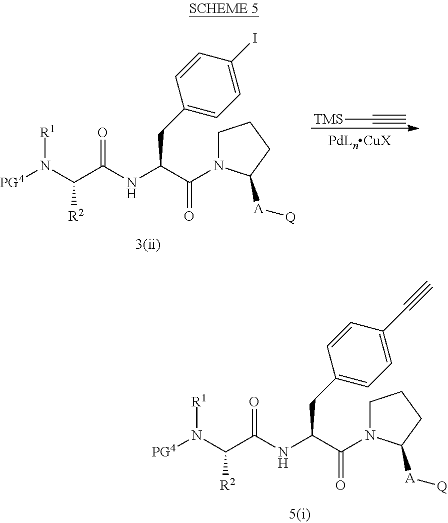

### General Procedure for the Coupling of Intermediate 1-6 and 1-4a

Scheme 6 illustrates a general procedure for the preparation of compounds of formula 6(ii). Coupling of intermediates 2(iii) and 3(ii) with an appropriate Pd catalyst system provides intermediate 6(i). Deprotection of protecting groups PG¹ and PG², either in a single or multiple steps, provides compounds of formula 6(ii).

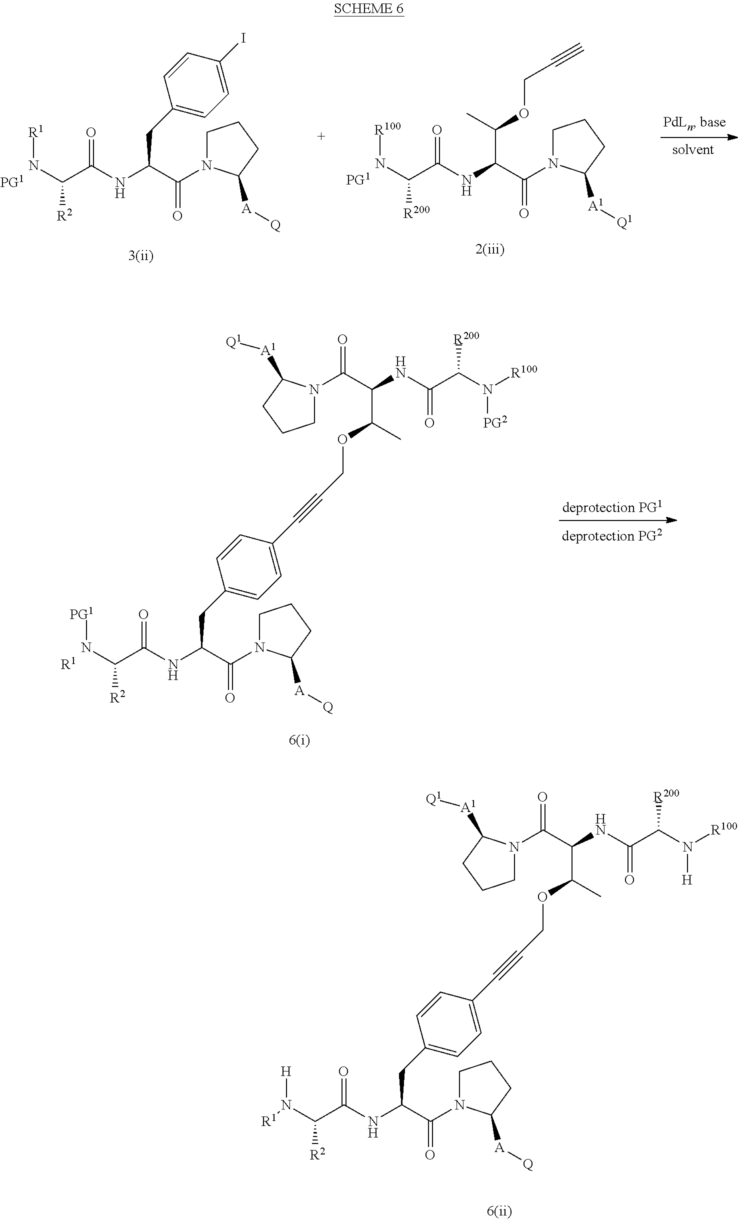

### General Procedure for the Preparation of Compounds of Formula 7(ii)

Scheme 7 illustrates a general procedure for the preparation of compounds of formula 7(ii). Coupling of intermediates 3(iii) and 4(ii) with an appropriate Pd catalyst system provides intermediate 7(i). Deprotection of protecting groups PG¹ and PG², either in a single or multiple steps, provides compounds of formula 7(ii).

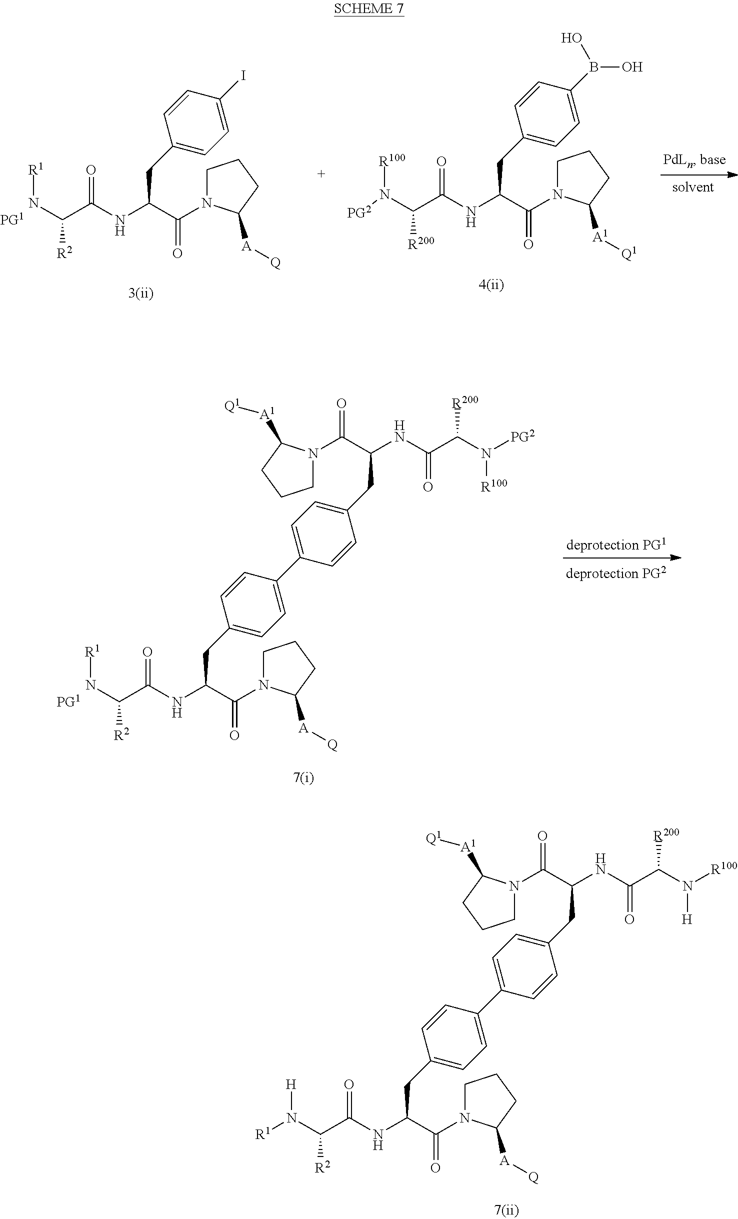

### General Procedure for the Preparation of Compounds of Formula 8(ii)

Scheme 8 illustrates a general procedure for the preparation of compounds of formula 8(ii). Homo-coupling of intermediate 5(i) with copper (i) chloride provides intermediate 8(i). Deprotection of protecting groups PG¹ provides compounds of formula 8(ii).

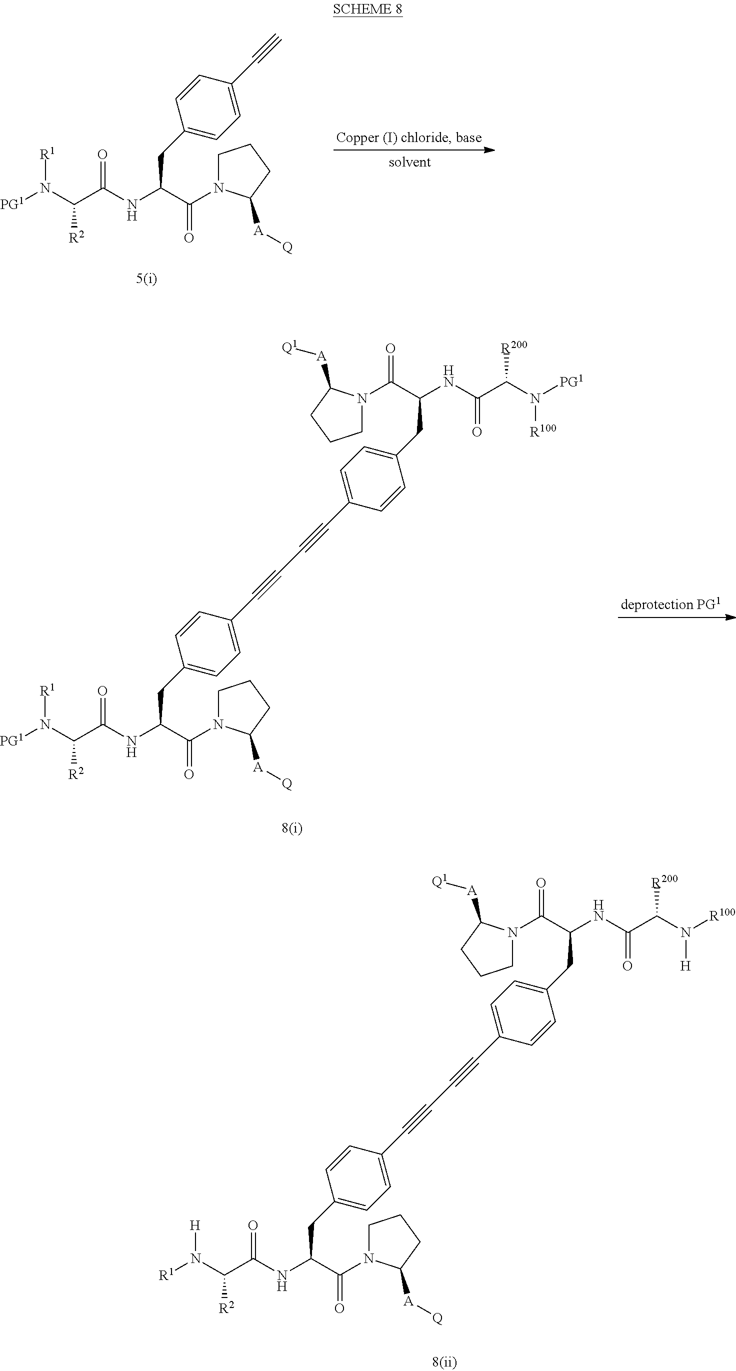

The above Schemes are applicable to both symmetrical compounds and unsymmetrical compounds, according to several embodiments. The substituents A¹, A, Q, Q¹, R¹, R¹⁰⁰, R², R²⁰⁰, R³, R³⁰⁰, and the like are as defined herein.

### EXAMPLES

The following abbreviations are used throughout:
Boc: t-butoxycarbonyl;
CBz: benzyloxycarbonyl;
DCM: dichloromethane;
DIPEA: diisopropylethylamine;
DMAP: 4-(dimethylamino)pyridine;
DMF: N,N-dimethylformamide;
DTT: dithiothreitol;
EDC: 3-dimethylaminopropyl)-3-ethylcarbodiimide hydrochloride;
EDTA: ethylenediaminetetracetic acid;
Fmoc: N-(9-fluorenylmethoxycarbonyl);
HBTU: O-(benzotriazol-1-yl)-N,N,N′,N′-tetramethyluronium hexafluorophosphate;
HCl: hydrochloric acid;
HOAc: acetic acid;
HOBt: 1-hydroxybenzotriazole;
HPLC: high performance liquid chromatography;
LCMS: liquid chromatography-mass spectrometer;
MeOH: methanol;
MgSO₄: magnesium sulfate;
MS: mass spectrum;
NaHCO₃: sodium hydrogen carbonate;
Pd/C: palladium on carbon;
TEA: triethylamine; and
THF: tetrahydrofuran.

### 1. Synthesis of Compound 1

### Step One:

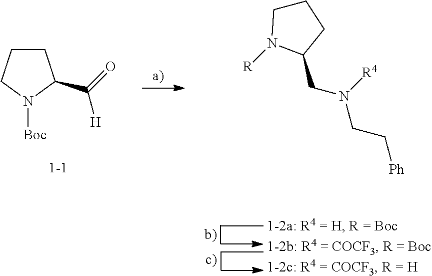

### Step A)

To a solution of N-(tert-butoxycarbonyl)-L-prolinal, 1-1 (6.00 g, 30.1 mmol), in methylene chloride was added phenethylamine (3.8 mL, 30.1 mmol). After stirring for 1 hr at room temperature sodium cyanoborohydride (12.8 g, 60.2 mmol) was added and the reaction mixture was stirred at room temperature overnight. Aqueous NaHCO₃ and ethyl acetate were added, the organic layer was separated, washed with brine, dried over MgSO₄ and concentrated in vacuo. Purification by flash chromatography provides 1-2a as colorless oil. MS (m/z) M+1=305.2

### Step B)

To a solution of 1-2a (6.0 g, 19.7 mmol) in methylene chloride were sequentially added triethylamine (5.5 mL, 39.5 mmol), 4-dimethylamino pyridine (catalytic) and trifluoroacetic anhydride (4.2 mL, 29.6 mmol) and the reaction mixture was stirred for 3 h at room temperature. Aqueous NaHCO₃ and ethyl acetate were added, the organic layer was separated, washed with brine, dried over MgSO₄ and concentrated in vacuo. Purification by flash chromatography provides 1-2b as colorless oil.

### Step C)

A 4 N solution of HCl in 1,4-dioxane (20 mL) was added to I-2b (7.4 g, 18.5 mmol) at room temperature and the solution was stirred for 2 h and then concentrated in vacuum. Crystallization from ether provides 1-2c as a white solid. MS (m/z) M+1=301.2

### Step Two—Synthesis of 1-2d

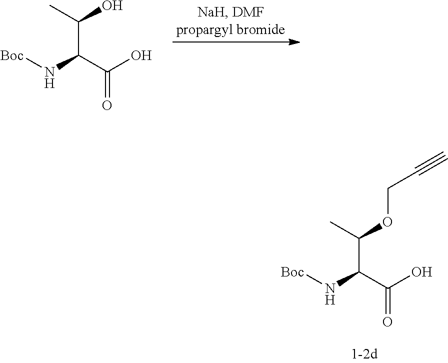

To a suspension of NaH (4.56 g, 114.04 mmol) in dry DMF (100 mL) cooled to 0° C. was added portion wise N-Boc-L-threonine (10.00 g, 45.62 mmol). After stirring for 10 min propargyl bromide (10 mL) was slowly added and the reaction was stirred for 1 hr at 0° C. Water (500 mL) and ethyl acetate (100 mL) were added, the organic layer was separated, the aqueous layer was acidified to pH=5 with 10% citric acid and extracted twice with ethyl acetate. The combined organic extracts were washed with brine, dried over MgSO₄ and concentrated in vacuo. Purification by flash chromatography provides 1-2d as colorless oil.

### Step Three

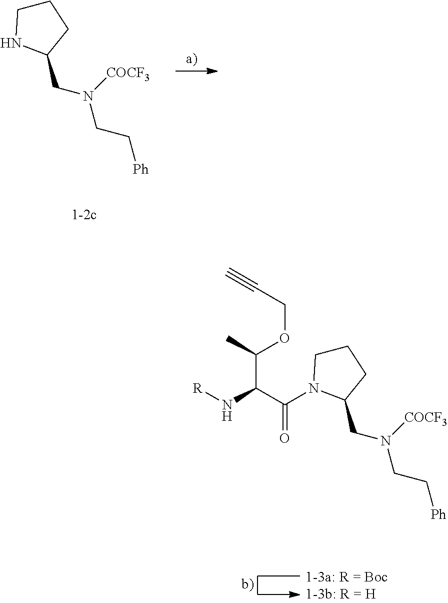

### Step A)

To a solution of 1-2d (7.2 g, 21.3 mmol) in DMF were sequentially added DIPEA (19.0 mL, 106 mmol), HOBt (4.24 g, 27.7 mmol) and HBTU (10.5 g, 27.7 mmol). After stirring for 5 min 1-2c (7.1 g, 27.7 mmol) was added and the reaction mixture was stirred overnight at room temperature. Water and ethyl acetate were added, the organic layer was separated, washed with 10% citric acid, aqueous NaHCO₃ and brine, dried over MgSO₄ and concentrated in vacuo. Purification by flash chromatography provides 1-3a as a white solid.

### Step B)

A 4 N solution of HCl in 1,4-dioxane (15 mL) was added to 1-3a (10.7 g, 18.0 mmol) at room temperature and the solution was stirred for 2 hrs and then concentrated in vacuo. Crystallization from ether provides 1-3b as a white solid. MS (m/z) M+1=440.2

### Step Four

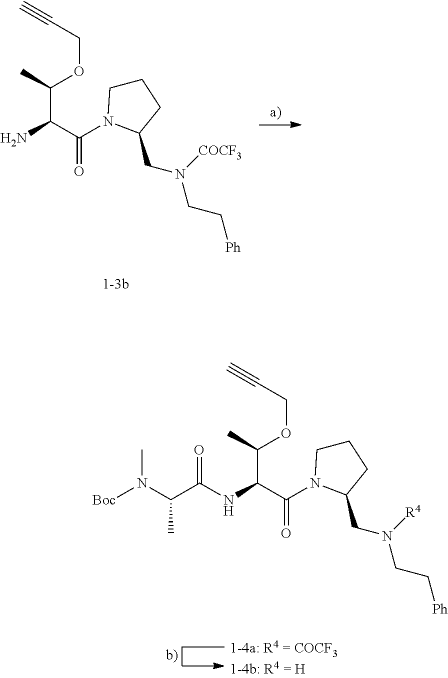

### Step A)

To a solution of 1-3b (8.9 g, 18.7 mmol) in DMF were sequentially added DIPEA (16.7 mL, 93.6 mmol), HOBt (3.7 g, 24.3 mmol) and HBTU (9.2 g, 24.3 mmol). After stirring for 5 min Boc-NMeAlaOH (4.9 g, 24.3 mmol) was added and the reaction mixture was stirred overnight at room temperature. Water and ethyl acetate were added, the organic layer was separated, washed with 10% citric acid, aqueous NaHCO₃ and brine, dried over MgSO₄ and concentrated in vacuo. Purification by flash chromatography provides 1-4a as a white solid.

### Step B)

To a solution of 1-4a (8.7 g, 13.4 mmol) in THF cooled to 0° C. was added 2N LiOH (20 mL) and the reaction was stirred overnight at room temperature. PH was adjusted to 6 with 10% citric acid and ethyl acetate was added, the organic layer was separated, washed with brine dried over MgSO₄ and concentrated in vacuo. Purification by flash chromatography provides 1-4b as a white solid. MS (m/z) M+1=625.0

### Step Five

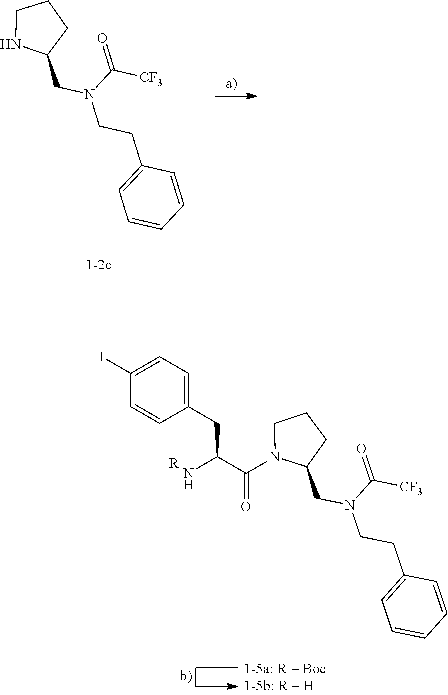

### Step A)

To a solution of Boc-4-iodo-phenylalanine (2.66 g, 6.80 mmol) in DMF were sequentially added DIPEA (7.3 mL, 41.9 mmol), HOBt (1.04 g, 7.70 mmol) and HBTU (2.49 g, 6.57 mmol). After stirring for 5 min 1-2c (2.08 g, 6.18 mmol) was added and the reaction mixture was stirred overnight at room temperature. Water and ethyl acetate were added, the organic layer was separated, washed with 10% citric acid, aqueous NaHCO₃ and brine, dried over MgSO₄ filtered and concentrated in vacuo. Purification by silica gel chromatography eluting with a 95:5 to 50:50 hexane/ethyl acetate gradient provided 1-5a as colorless oil.

### Step B)

4N HCl in 1,4-dioxane (15 mL) was added to 1-5a (3.4 g, 5.05 mmol) and the solution was stirred for 2 hrs at room temperature. Volatiles were removed under reduced pressure and the residue was triturated with diethyl ether to provide 1-5b as a yellow solid. MS: (m/z) M+1=574.2

### Step Six

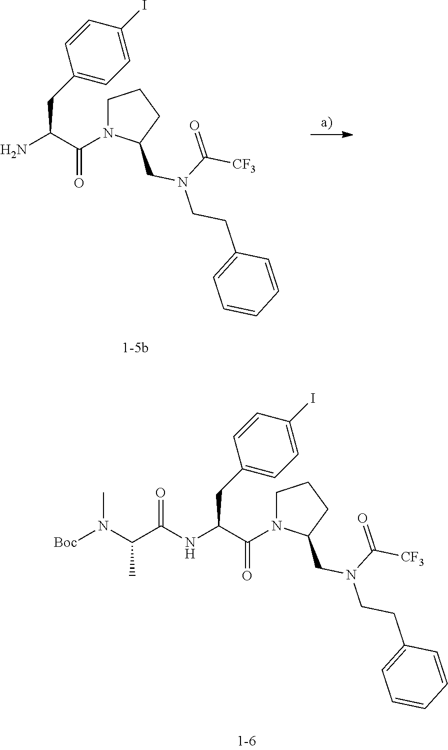

### Step A)

To a solution of Boc-NMe-Ala-OH (1.12 g, 5.51 mmol) in DCM were sequentially added DIPEA (6.0 mL, 34.4 mmol), HOBt (866 mg, 6.41 mmol) and EDC (1.00 g, 5.22 mmol). After stirring for 5 min 1-5b (3.02 g, 4.92 mmol) was added and the reaction mixture was stirred overnight at room temperature. Water and ethyl acetate were added, the organic layer was separated, washed with 10% citric acid, aqueous NaHCO₃ and brine, dried over MgSO₄ filtered and concentrated in vacuo. Purification by silica gel chromatography eluting with a 95:5 to 50:50 hexane/ethyl acetate gradient provided 1-6 as a white solid.

### Step Seven—Compound 1

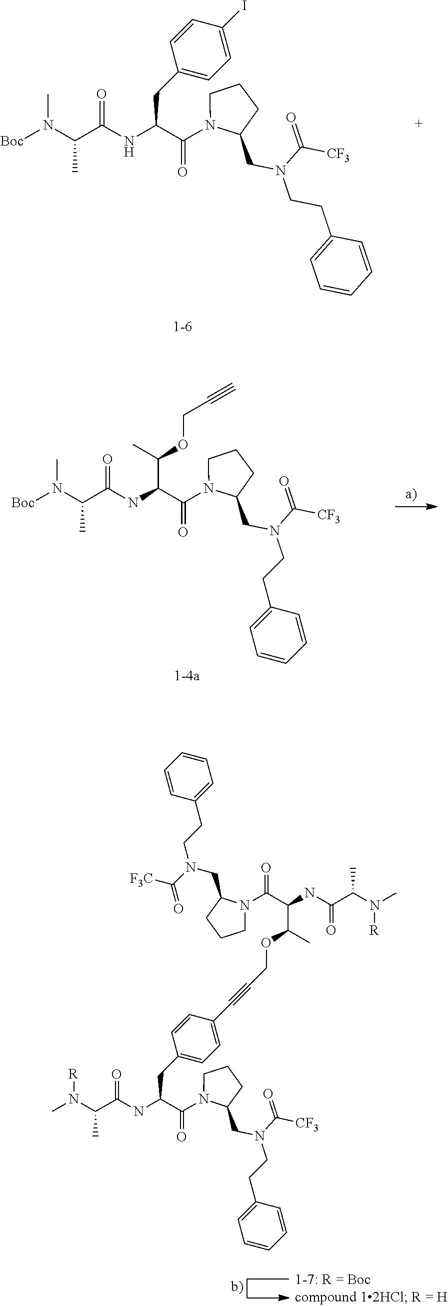

### Step A)

To a solution of 1-6 (123 mg, 0.16 mmol) and 1-4a (170 mg, 0.27 mmol) in triethylamine (4.0 mL) was added PdCl₂(dppf) (2.0 mg, 0.0028 mmol) and CuI (4.7 mg, 0.025 mmol) and the reaction was stirred overnight at room temperature. Water and ethyl acetate were added, the organic layer was separated, washed with 10% citric acid, aqueous NaHCO₃ and brine, dried over MgSO₄ filtered and concentrated in vacuo. Purification by silica gel chromatography eluting with a 95:5 to 50:50 hexane/ethyl acetate gradient provided 1-7 as a white solid.

### Step B)

4N HCl in 1,4-dioxane (5.0 mL) was added to 1-7 (220 mg, 0.175 mmol) and the solution was stirred for 2 hrs at 0° C. Volatiles were removed under reduced pressure and the residue was triturated with diethyl ether to provide compound 1.2HCl as a white solid. MS: (m/z) M+1=1055.6

### 2. Synthesis of Compound 2

### Step One

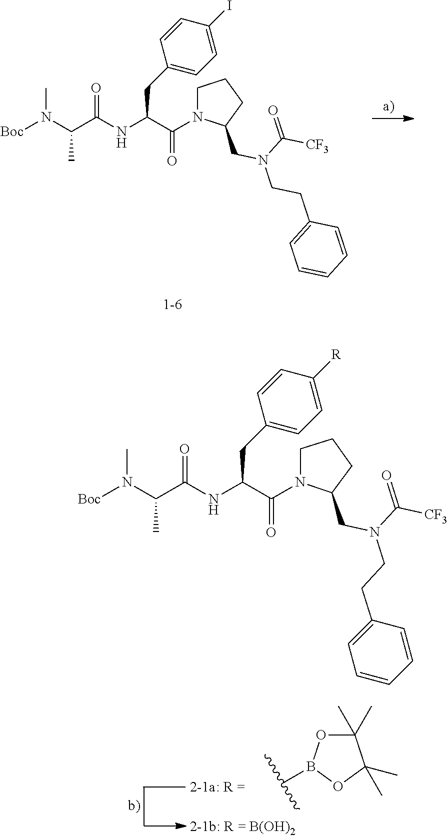

### Step A)

To a solution of 1-6 (411 mg, 0.52 mmol) in DMF was added potassium acetate (160 mg, 1.63 mmol), pinacol diboron (151 mg, 0.59 mmol), and PdCl₂(dppf) (20.2 mg, 0.027 mmol). The mixture was heated in a sealed tube for 18 hrs and then cooled to room temperature. Water and ethyl acetate were added, the organic layer was separated, washed with brine, dried over MgSO₄ filtered and concentrated in vacuo. Purification by silica gel chromatography eluting with a 95:5 to 20:80 hexane/ethyl acetate gradient provided 2-1a as a white solid.

### Step B)

To a solution of 2-1a (287 mg, 0.378 mmol) in acetone (21.6 ml) and water (16.0 ml) was added ammonium acetate (119 mg, 1.54 mmol), and sodium periodate (314 mg, 1.47 mmol) and the reaction was stirred at room temperature for 18 hrs. Water and ethyl acetate were added, the organic layer was separated, washed with brine, dried over MgSO₄ filtered and concentrated in vacuo. Purification by silica gel chromatography eluting with a 95:5 to 20:80 hexane/ethyl acetate gradient provided 2-1b as a white solid.

### Step Two—Compound 2

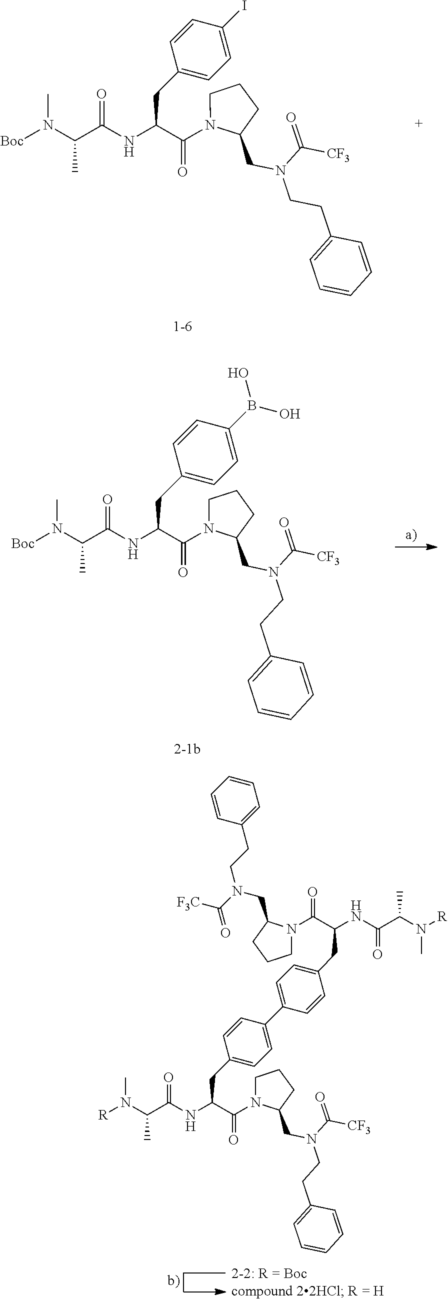

### Step A)

To a solution of 1-6 (59 mg, 0.078 mmol) and 2-1b (58 mg, 0.086 mmol) in DMF were added PdCl₂(dppf) (1.6 mg, 0.0023 mmol) and NaHCO₃ (15.8 mg, 0.149 mmol) and the reaction was stirred overnight at room temperature. Water and ethyl acetate were added, the organic layer was separated, washed with 10% citric acid, aqueous NaHCO₃ and brine, dried over MgSO₄ filtered and concentrated in vacuo. Purification by silica gel chromatography eluting with a 95:5 to 5:95 hexane/ethyl acetate gradient provided 2-2 as a white solid.

### Step B)

4N HCl in 1,4-dioxane (3 mL) was added to 2-2 (30 mg, 0.024 mmol) and the solution was stirred for 2 hrs at room temperature. Volatiles were removed under reduced pressure and the residue was triturated with diethyl ether to provide compound 2.2HCl as a white solid. MS: (m/z) (M+2)/2=532.4.

### 3. Synthesis of Compound 3

### Step One

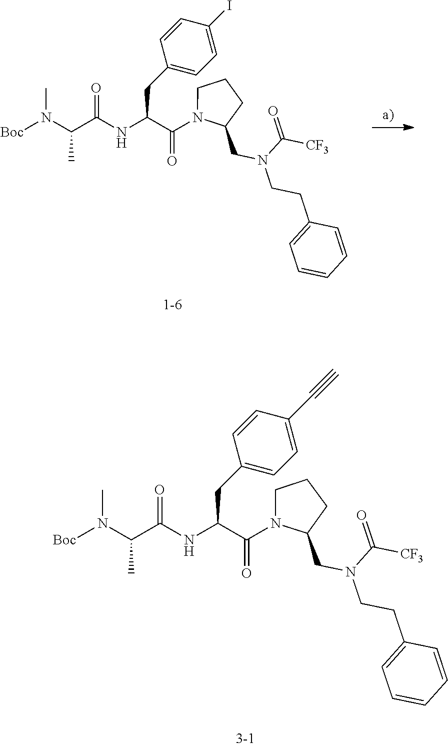

### Step A)

To a solution of 1-6 (310 mg, 0.41 mmol) in trietylamine (4.0 mL) was added PdCl₂(dppf) (3.9 mg, 0.0056 mmol), CuI (3.0 mg, 0.016 mmol) and trimethylsilylacetylene (100 μL, 0.708 mmol) and the reaction was stirred overnight at room temperature. Water and ethyl acetate were added, the organic layer was separated, washed with 10% citric acid, aqueous NaHCO₃ and brine, dried over MgSO₄ filtered and concentrated in vacuo. The residue was dissolved in anhydrous THF (10 ml) and cooled to 0° C. A 1M solution of Tetrabutylammonium fluoride (0.80 ml, 0.80 mmol) was then added and the mixture was stirred for 2 hrs. Water and ethyl acetate were added, the organic layer was separated, washed with 10% citric acid, aqueous NaHCO₃ and brine, dried over MgSO₄ filtered and concentrated in vacuo. Purification by silica gel chromatography eluting with a 95:5 to 50:95 hexane/ethyl acetate gradient provided 3-1 as colorless oil.

### Step Two

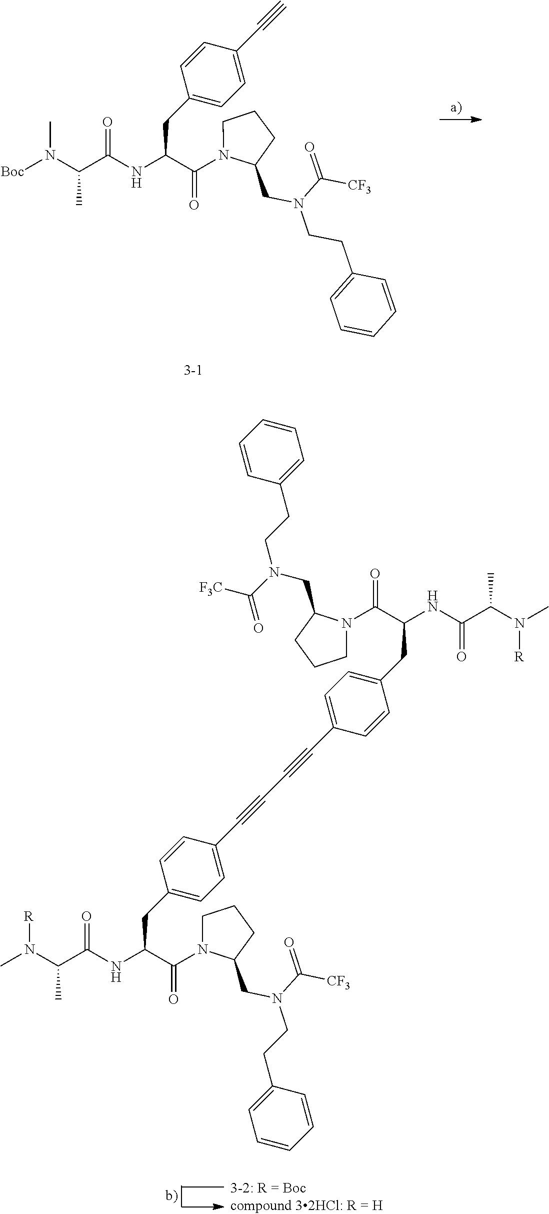

### Step A)

To a solution of 3-1 (160 mg, 0.24 mmol) in dry acetone were sequentially added tetramethylethylenediamine (75 uL, 0.50 mmol) and copper (I) chloride (49.8 mg, 0.5 mmol), the reaction was then stirred overnight at room temperature and solvent was removed in vacuo. Water and ethyl acetate were added, the organic layer was separated, washed with 10% citric acid, aqueous NaHCO₃ and brine, dried over MgSO₄ filtered and concentrated in vacuo. Purification by silica gel chromatography eluting with a 95:5 to 50:50 hexane/ethyl acetate gradient provided 3-2 as a white solid.

### Step B)

4N HCl in 1,4-dioxane (4.0 mL) was added to 3-2 (80 mg, 0.06 mmol) and the solution was stirred for 3 hrs at 0° C. Volatiles were removed under reduced pressure and the residue was triturated with diethyl ether to provide compound 3.2HCl as a white solid. MS: (m/z) (M+2)/2=556.4.

### 4. Synthesis of Intermediate 4-1

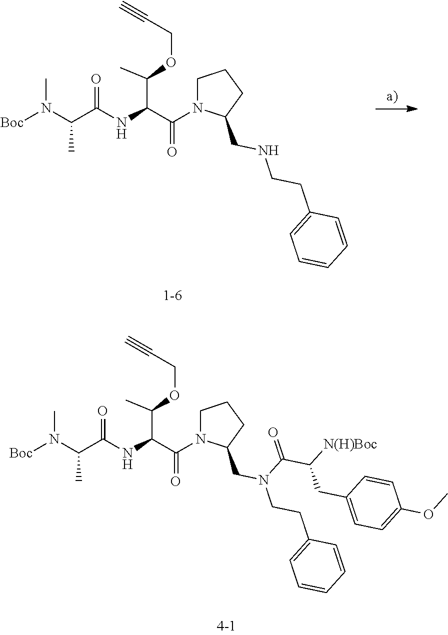

### Step One

To a solution of 1-6 (900 mg, 1.7 mmol) in DMF were sequentially added DIPEA (1.5 mL, 8.5 mmol), HBTU (841 mg, 2.2 mmol) and HOBt (340 mg, 2.2 mmol). After stirring for 5 min Boc-D-Tyr(Me)—OH (655 mg, 2.2 mmol) was added and the reaction mixture was stirred overnight at room temperature. Water and ethyl acetate were added, the organic layer was separated, washed with 10% citric acid, aqueous NaHCO₃ and brine, dried over MgSO₄ and concentrated in vacuo. Purification by flash chromatography provides 4-1 as a white solid.

Representative embodiments of compounds were prepared by simple modification of the above procedures and are illustrated in Table 1:

TABLE 1

| Compound | Structure | MS (m/z) |
|---|---|---|
| 1 | 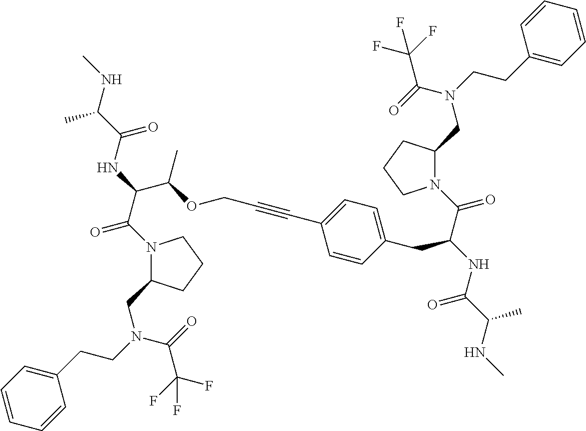 | M + 1 = 1055.6 (M + 2)/2 = 528.4 |
| 2 | 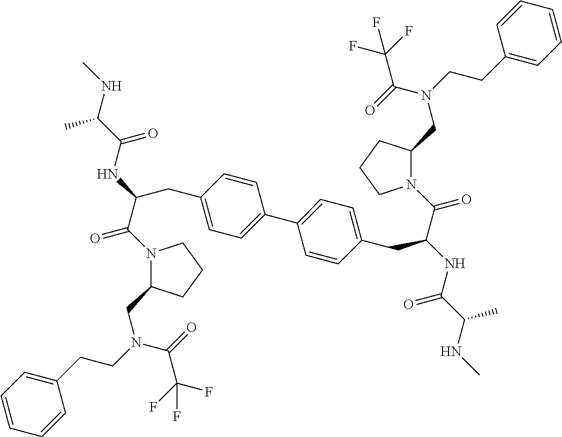 | (M + 2)/2 = 532.4 |
| 3 | 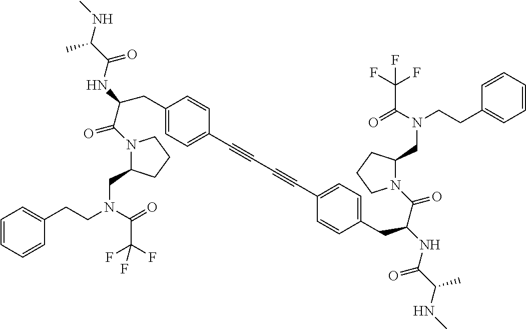 | (M + 2)/2 = 556.4 |
| 4 | 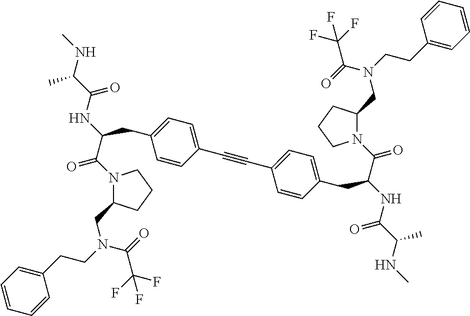 | (M + 2)/2 = 544.4 |
| 5 | 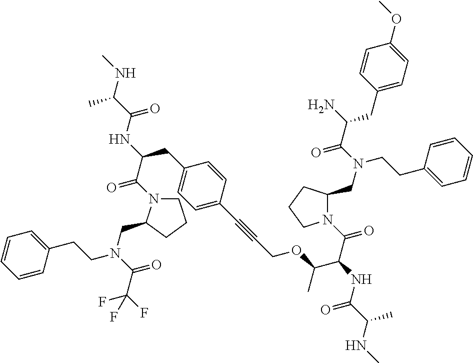 | (M + 2)/2 = 569.0 |
| 6 | 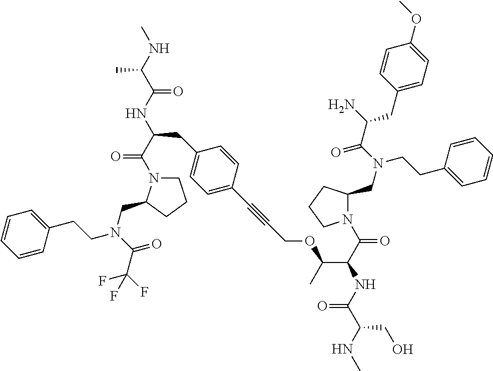 | 410 (M + 2)/2 = 576.8 |
| 7 | 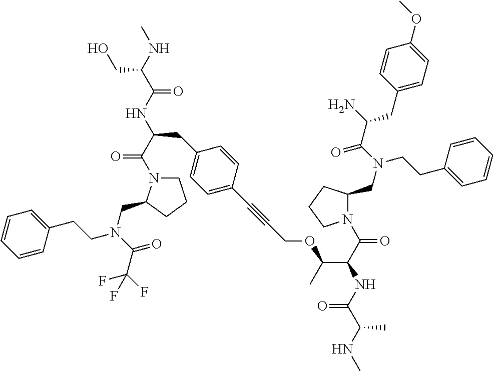 | (M + 2)/2 = 576.8 |
| 8 | 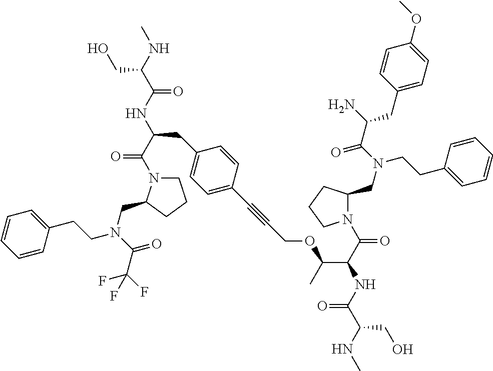 | (M + 2)/2 = 585.0 |

Representative embodiments of compounds that can be prepared by simple modification of the above procedures are illustrated in Tables 2 through 5:

TABLE 2
M1-BG-M2
FORMULA lA
BG is

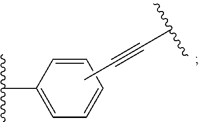

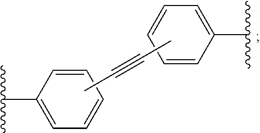

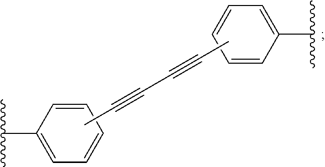

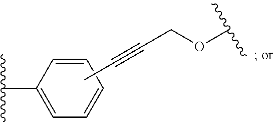

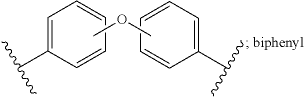

biphenyl

| M1 | M2 |
|---|---|
| 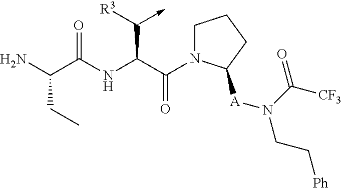 | 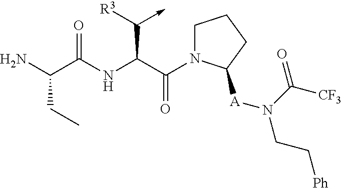 |
| A = CH₂; R³ = H or Me | A = CH₂; R³ = H or Me |
| 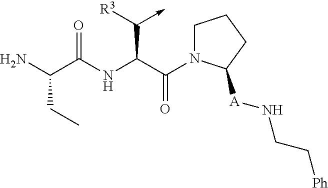 | 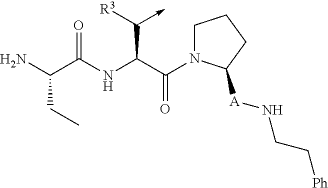 |
| A = CH₂; R³ = H or Me | A = CH₂; R³ = H or Me |
| 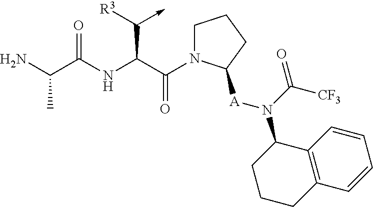 | 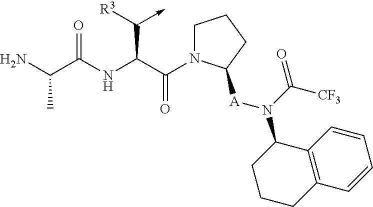 |
| A = CH₂; R³ = H or Me | A = CH₂; R³ = H or Me |
| 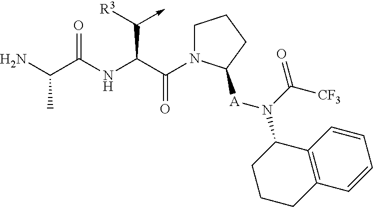 | 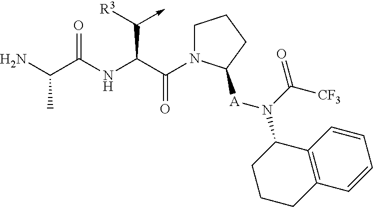 |
| A = CH₂; R³ = H or Me | A = CH₂; R³ = H or Me |
| 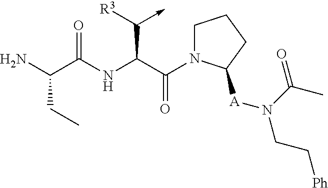 | 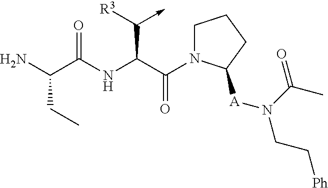 |
| A = CH₂; R³ = H or Me | A = CH₂; R³ = H or Me |
| 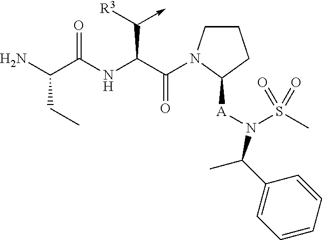 | 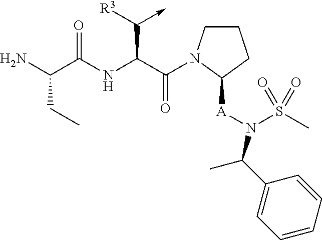 |
| A = CH₂; R³ = H or Me | A = CH₂; R³ = H or Me |
| 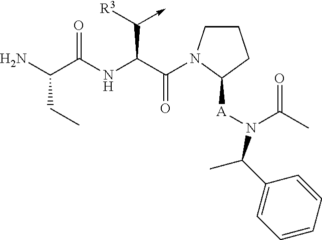 | 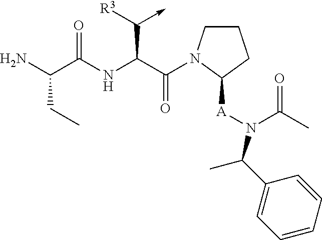 |
| A = CH₂; R³ = H or Me | A = CH₂; R³ = H or Me |
| 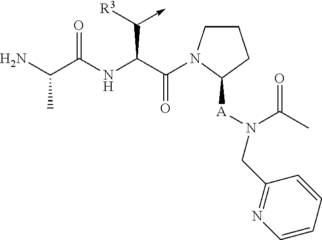 | 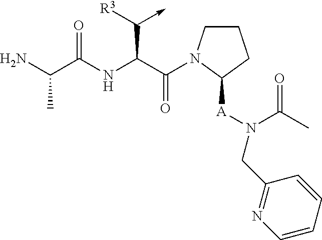 |
| A = CH₂; R³ = H or Me | A = CH₂; R³ = H or Me |
| 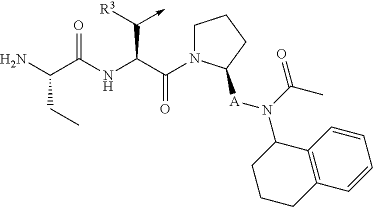 | 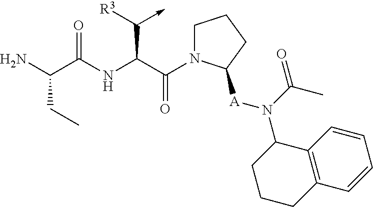 |
| A = CH₂; R³ = H or Me | A = CH₂; R³ = H or Me |
| 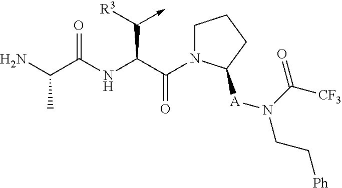 | 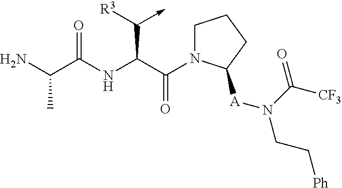 |
| A = CH₂; R³ = H or Me | A = CH₂; R³ = H or Me |
| 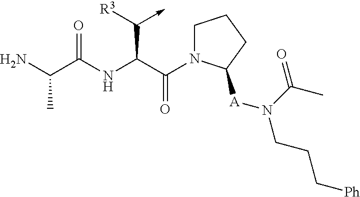 | 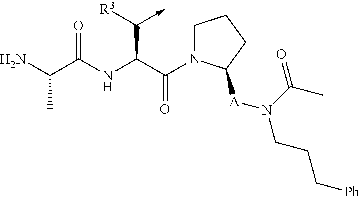 |
| A = CH₂; R³ = H or Me | A = CH₂; R³ = H or Me |
| 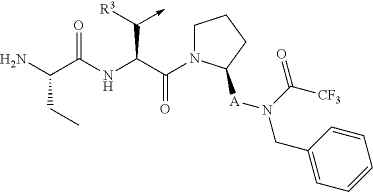 | 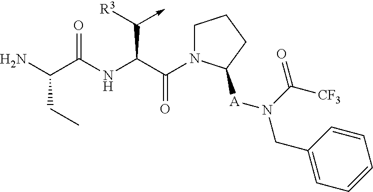 |
| A = CH₂; R³ = H or Me | A = CH₂; R³ = H or Me |
| 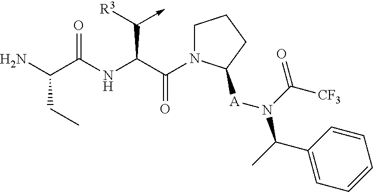 | 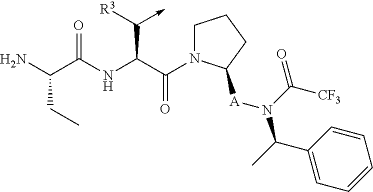 |
| A = CH₂; R³ = H or Me | A = CH₂; R³ = H or Me |
| 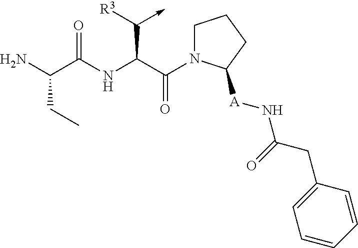 | 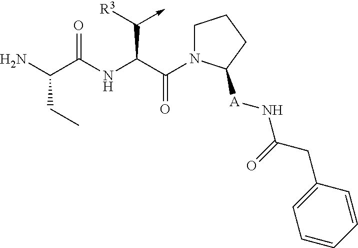 |
| A = CH₂; R³ = H or Me | A = CH₂; R³ = H or Me |
| 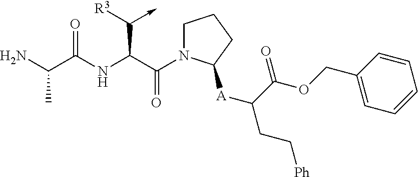 | 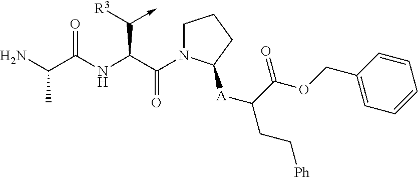 |
| A = CH₂; R³ = H or Me | A = CH₂; R³ = H or Me |
| 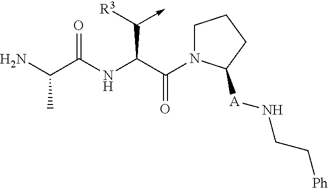 | 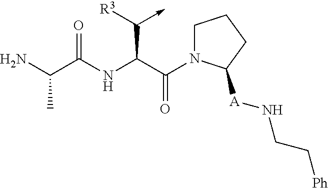 |
| A = CH₂; R³ = H or Me | A = CH₂; R³ = H or Me |
| 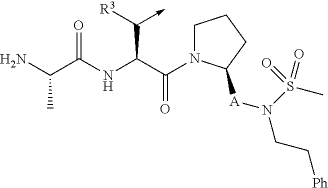 | 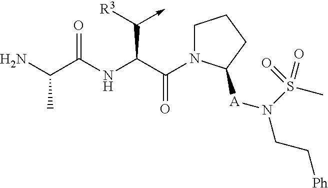 |
| A = CH₂; R³ = H or Me | A = CH₂; R³ = H or Me |
| 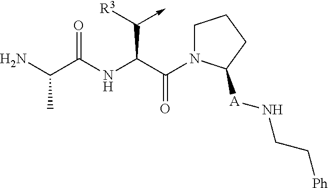 | 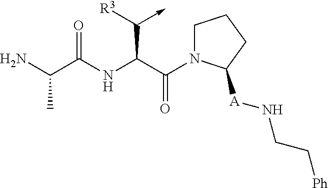 |
| A = CH₂; R³ = H or Me | A = CH₂; R³ = H or Me |
| 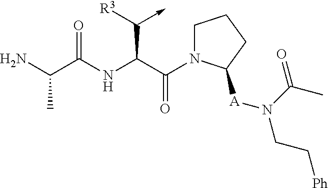 | 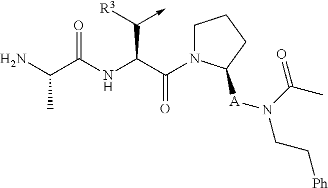 |
| A = CH₂; R³ = H or Me | A = CH₂; R³ = H or Me |
| 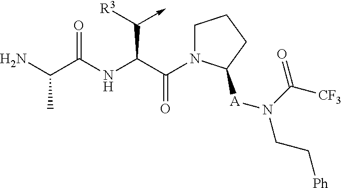 | 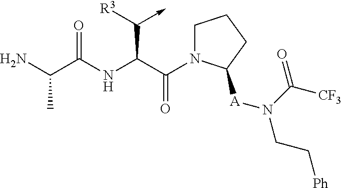 |
| A = CH₂; R³ = H or Me | A = CH₂; R³ = H or Me |
| 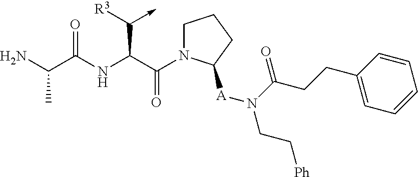 | 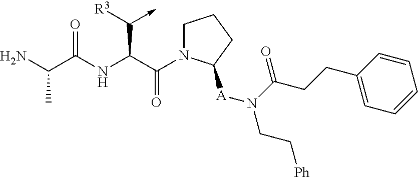 |
| A = CH₂; R³ = H or Me | A = CH₂; R³ = H or Me |
| 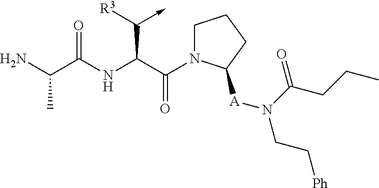 | 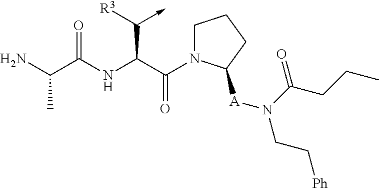 |
| A = CH₂; R³ = H or Me | A = CH₂; R³ = H or Me |
| 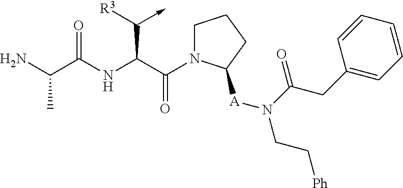 | 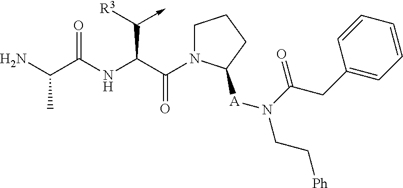 |
| A = CH₂; R³ = H or Me | A = CH₂; R³ = H or Me |
| 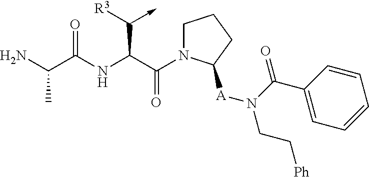 | 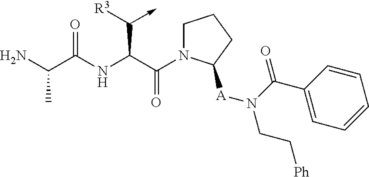 |
| A = CH₂; R³ = H or Me | A = CH₂; R³ = H or Me |
| 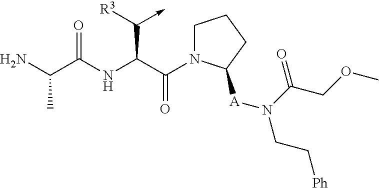 | 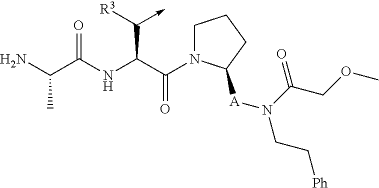 |
| A = CH₂; R³ = H or Me | A = CH₂; R³ = H or Me |
| 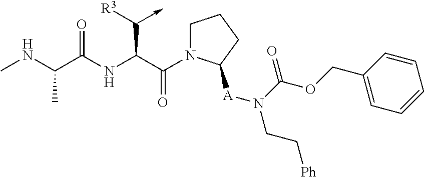 | 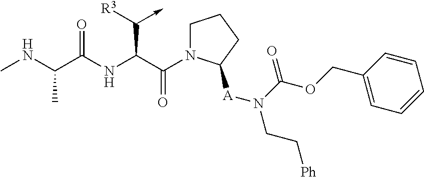 |
| A = CH₂; R³ = H or Me | A = CH₂; R³ = H or Me |
| 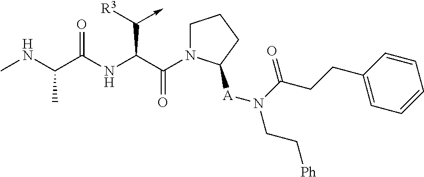 | 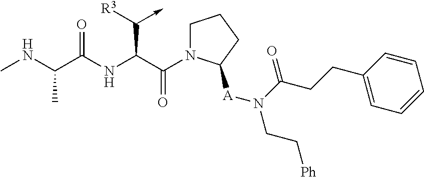 |
| A = CH₂; R³ = H or Me | A = CH₂; R³ = H or Me |
| 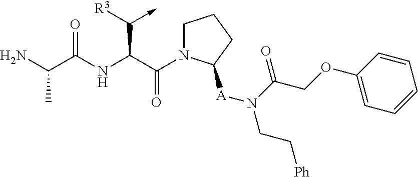 | 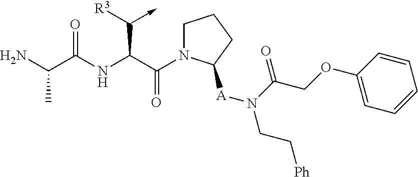 |
| A = CH₂; R³ = H or Me | A = CH₂; R³ = H or Me |
| 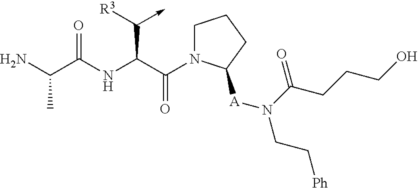 | 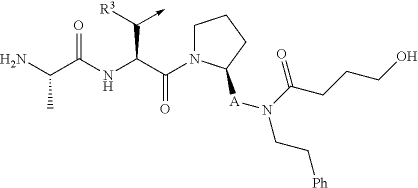 |
| A = CH₂; R³ = H or Me | A = CH₂; R³ = H or Me |
| 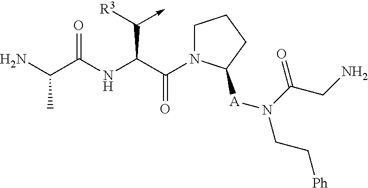 | 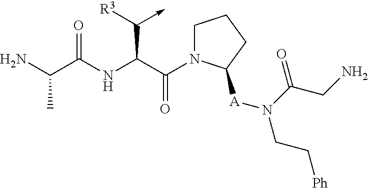 |
| A = CH₂; R³ = H or Me | A = CH₂; R³ = H or Me |
| 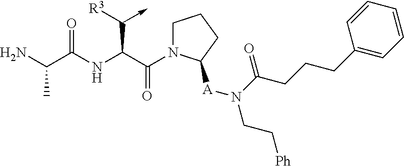 | 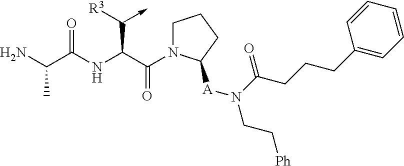 |
| A = CH₂; R³ = H or Me | A = CH₂; R³ = H or Me |
| 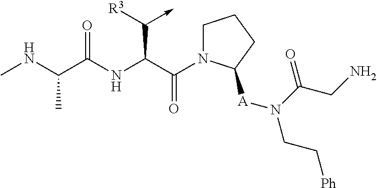 | 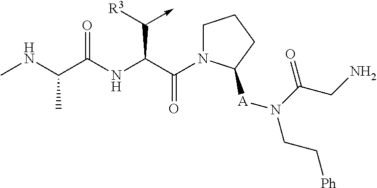 |
| A = CH₂; R³ = H or Me | A = CH₂; R³ = H or Me |
| 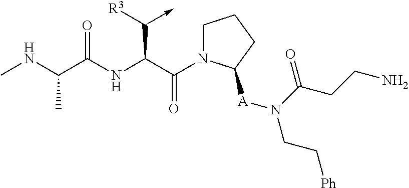 | 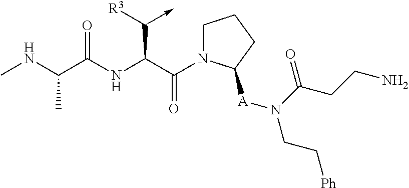 |
| 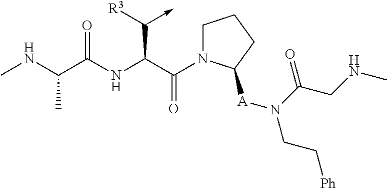 | 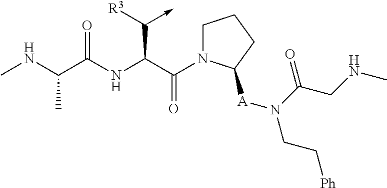 |
| A = CH₂; R³ = H or Me | A = CH₂; R³ = H or Me |
| 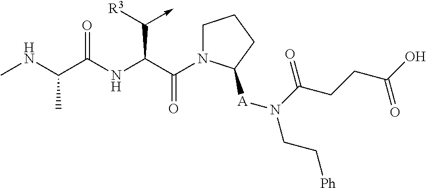 | 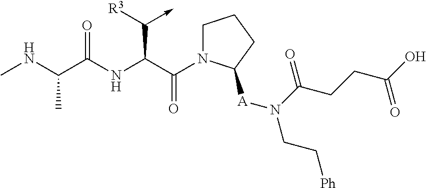 |
| A = CH₂; R³ = H or Me | A = CH₂; R³ = H or Me |
| 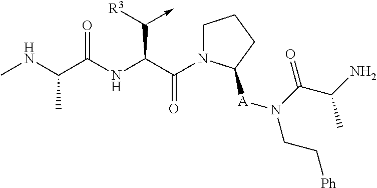 | 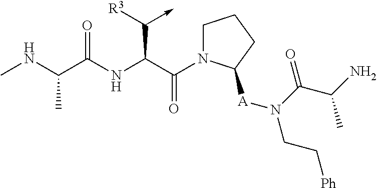 |
| A = CH₂; R³ = H or Me | A = CH₂; R³ = H or Me |
| 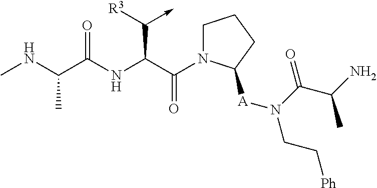 | 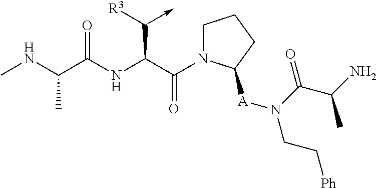 |
| A = CH₂; R³ = H or Me | A = CH₂; R³ = H or Me |
| 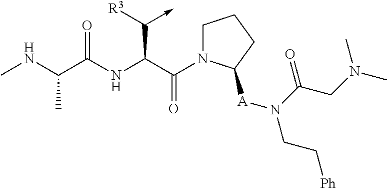 | 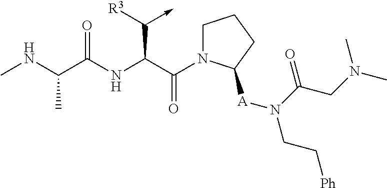 |
| A = CH₂; R³ = H or Me | A = CH₂; R³ = H or Me |
| 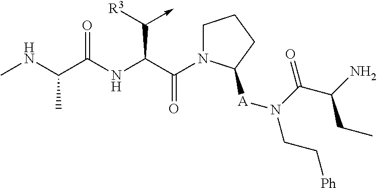 | 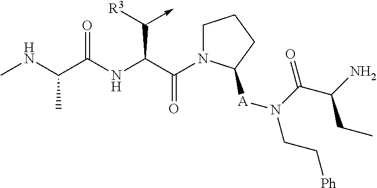 |
| A = CH₂; R³ = H or Me | A = CH₂; R³ = H or Me |
| 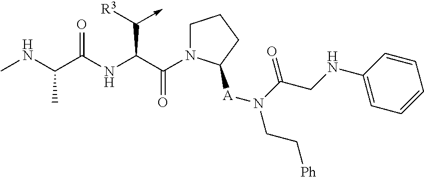 | 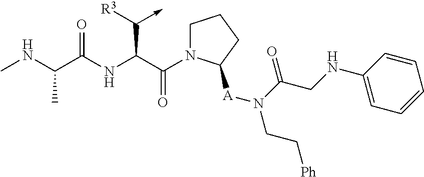 |
| A = CH₂; R³ = H or Me | A = CH₂; R³ = H or Me |
| 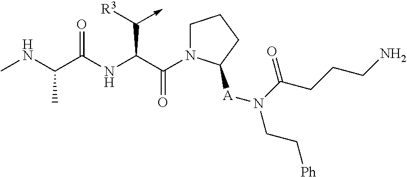 | 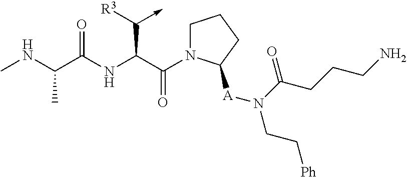 |
| A = CH₂; R³ = H or Me | A = CH₂; R³ = H or Me |
| 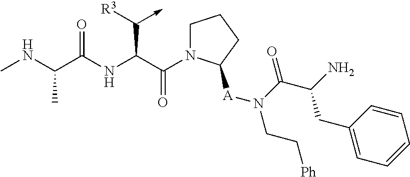 | 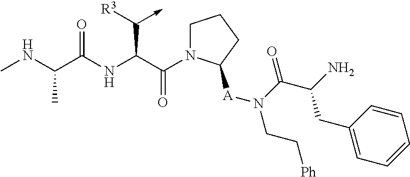 |
| A = CH₂; R³ = H or Me | A = CH₂; R³ = H or Me |
| 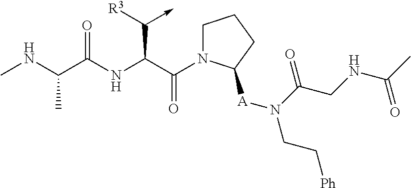 | 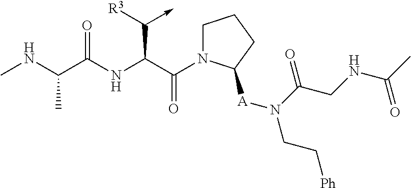 |
| A = CH₂; R³ = H or Me | A = CH₂; R³ = H or Me |
| 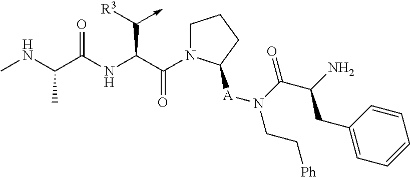 | 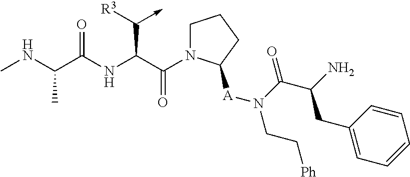 |
| A = CH₂; R³ = H or Me | A = CH₂; R³ = H or Me |
| 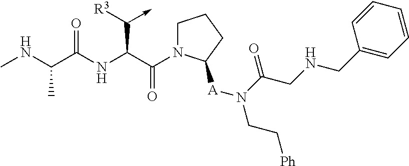 | 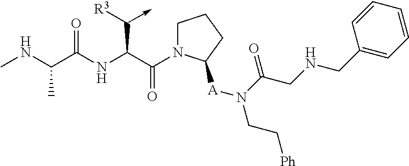 |
| A = CH₂; R³ = H or Me | A = CH₂; R³ = H or Me |
| 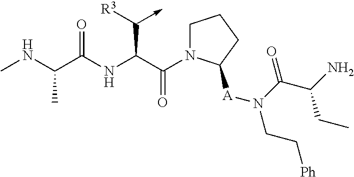 | 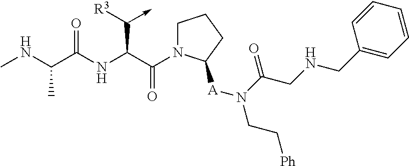 |
| A = CH₂; R³ = H or Me | A = CH₂; R³ = H or Me |
| 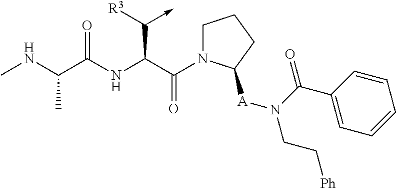 | 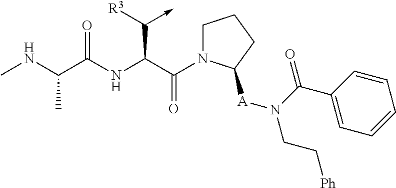 |
| A = CH₂; R³ = H or Me | A = CH₂; R³ = H or Me |
| 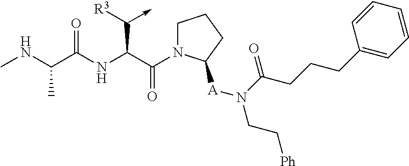 | 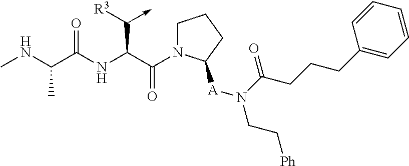 |
| A = CH₂; R³ = H or Me | A = CH₂; R³ = H or Me |
| 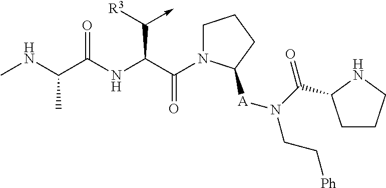 | 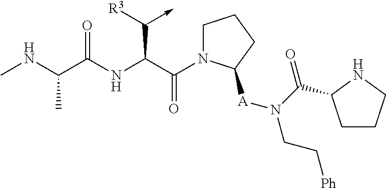 |
| A = CH₂; R³ = H or Me | A = CH₂; R³ = H or Me |
| 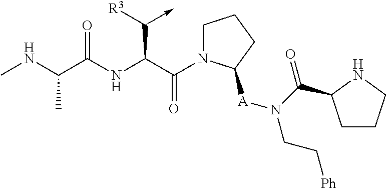 | 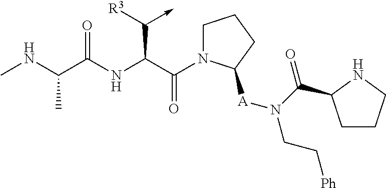 |
| A = CH₂; R³ = H or Me | A = CH₂; R³ = H or Me |
| 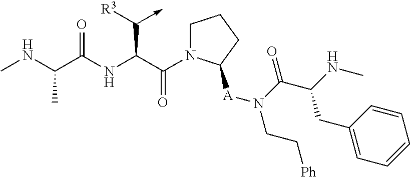 | 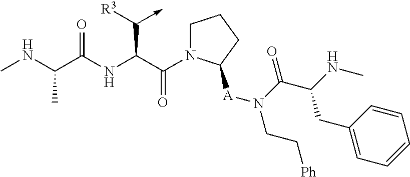 |
| A = CH₂; R³ = H or Me | A = CH₂; R³ = H or Me |
| 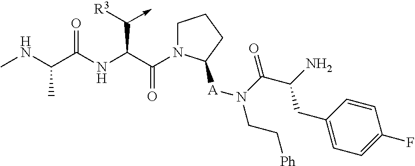 | 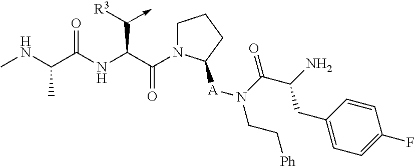 |
| A = CH₂; R³ = H or Me | A = CH₂; R³ = H or Me |
| 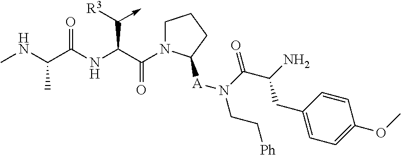 | 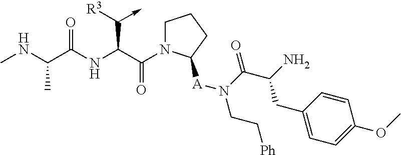 |
| A = CH₂; R³ = H or Me | A = CH₂; R³ = H or Me |
| 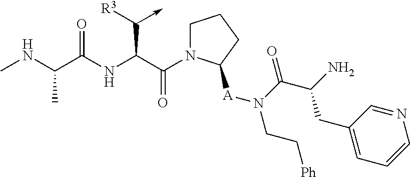 | 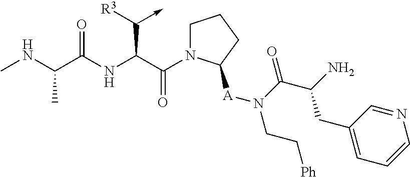 |
| A = CH₂; R³ = H or Me | A = CH₂; R³ = H or Me |
| 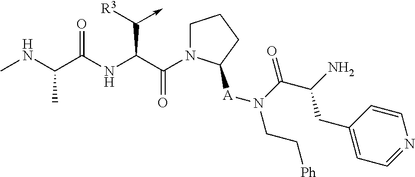 | 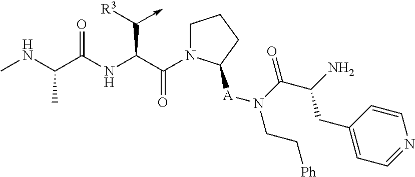 |
| A = CH₂; R³ = H or Me | A = CH₂; R³ = H or Me |
| 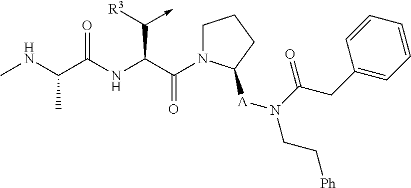 | 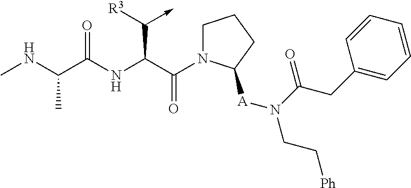 |
| A = CH₂; R³ = H or Me | A = CH₂; R³ = H or Me |
| 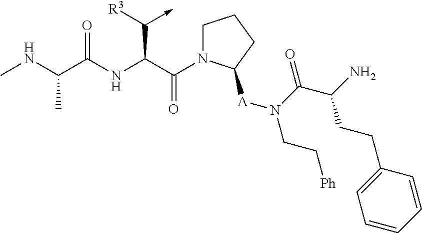 | 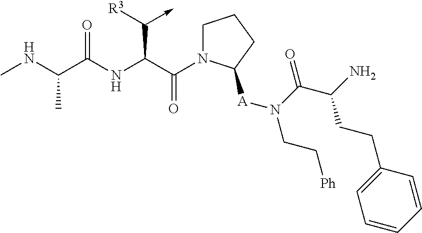 |
| A = CH₂; R³ = H or Me | A = CH₂; R³ = H or Me |
| 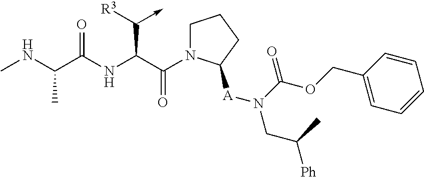 | 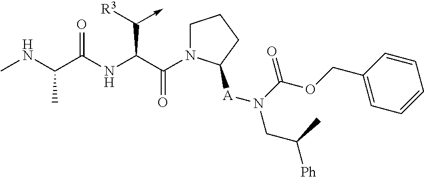 |
| A = CH₂; R³ = H or Me | A = CH₂; R³ = H or Me |
| 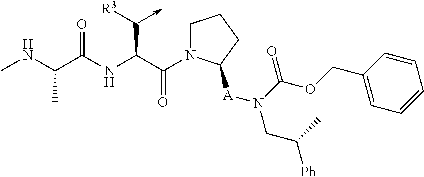 | 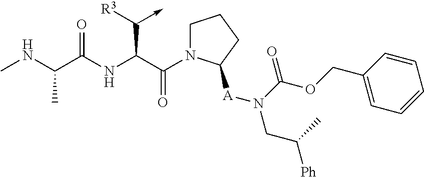 |
| A = CH₂; R³ = H or Me | A = CH₂; R³ = H or Me |
| 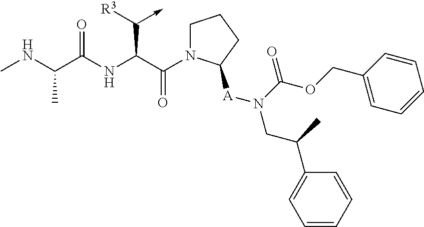 | 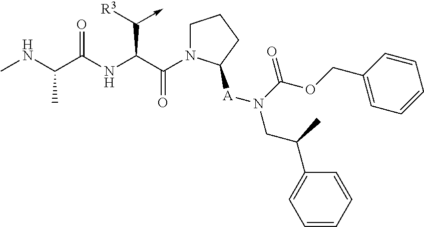 |
| A = CH₂; R³ = H or Me | A = CH₂; R³ = H or Me |
| 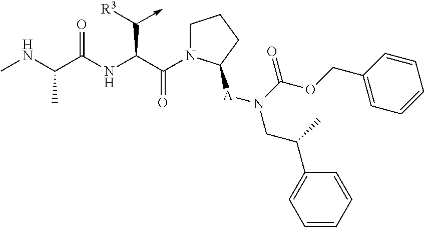 | 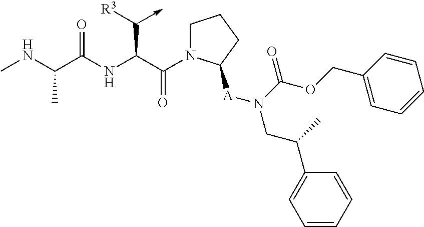 |
| A = CH₂; R³ = H or Me | A = CH₂; R³ = H or Me |
| 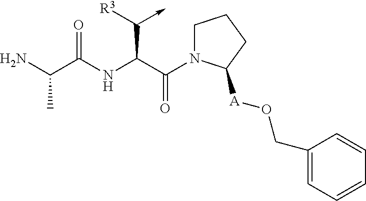 | 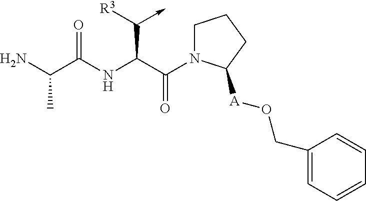 |
| A = CH₂; R³ = H or Me | A = CH₂; R³ = H or Me |
| 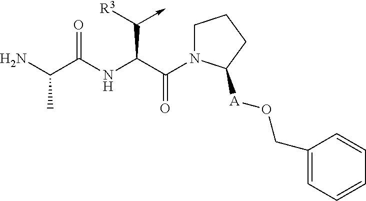 | 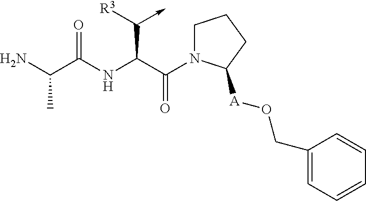 |
| A = CH₂; R³ = H or Me | A = CH₂; R³ = H or Me |
| 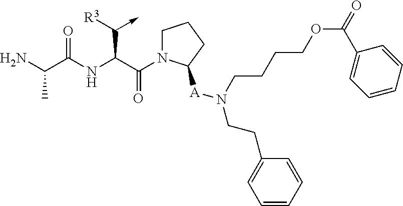 | 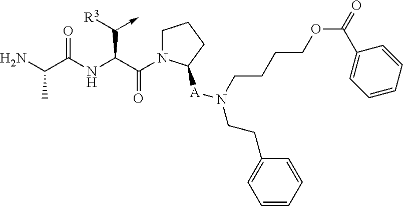 |
| A = CH₂; R³ = H or Me | A = CH₂; R³ = H or Me |
| 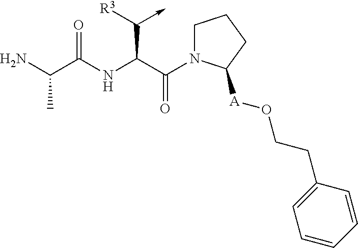 | 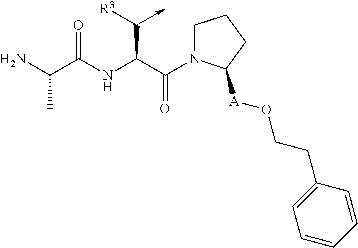 |
| A = CH₂; R³ = H or Me | A = CH₂; R³ = H or Me |
| 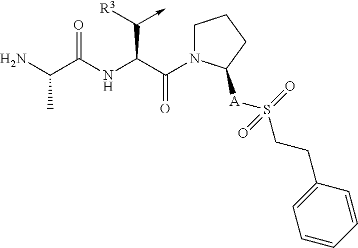 | 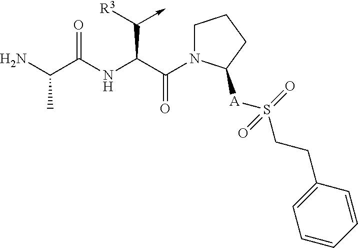 |
| A = CH₂; R³ = H or Me | A = CH₂; R³ = H or Me |
| 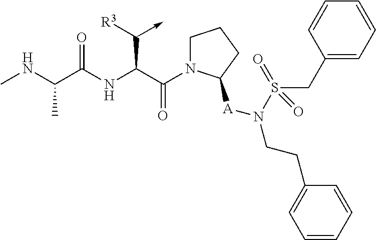 | 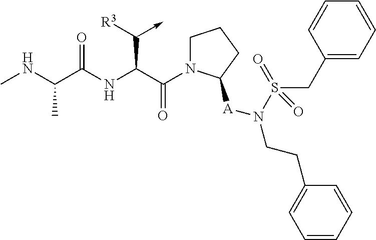 |
| A = CH₂; R³ = H or Me | A = CH₂; R³ = H or Me |
| 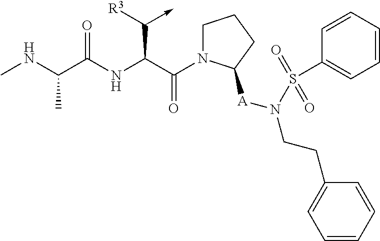 | 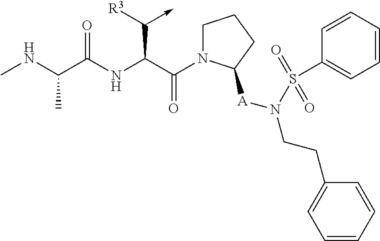 |
| A = CH₂; R³ = H or Me | A = CH₂; R³ = H or Me |
| 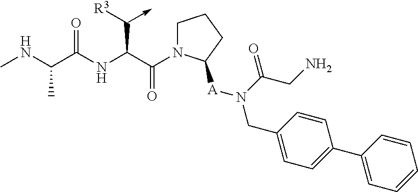 | 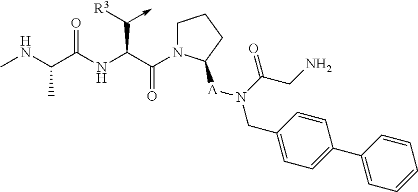 |
| A = CH₂; R³ = H or Me | A = CH₂; R³ = H or Me |
| 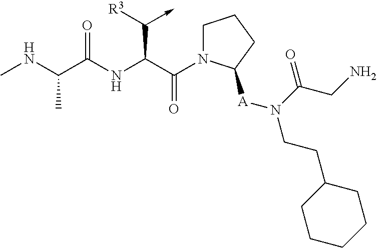 | 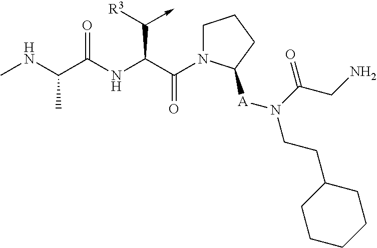 |
| A = CH₂; R³ = H or Me | A = CH₂; R³ = H or Me |
| 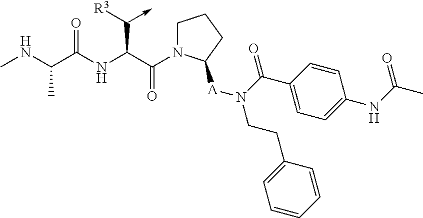 | 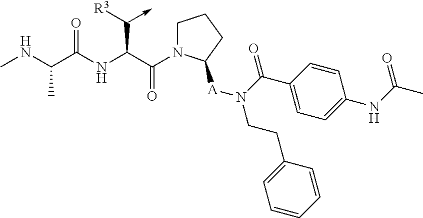 |
| A = CH₂; R³ = H or Me | A = CH₂; R³ = H or Me |
| 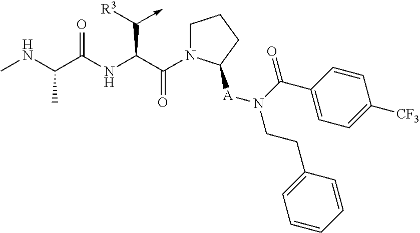 | 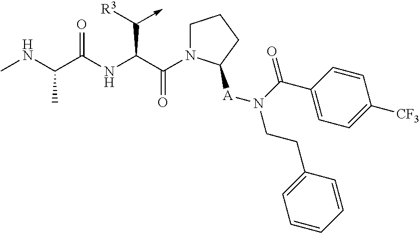 |
| A = CH₂; R³ = H or Me | A = CH₂; R³ = H or Me |
| 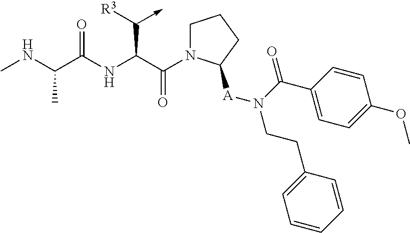 | 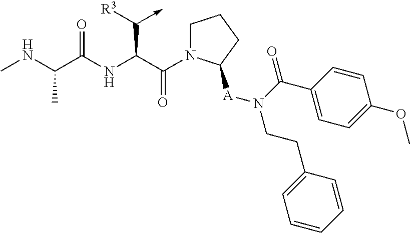 |
| A = CH₂; R³ = H or Me | A = CH₂; R³ = H or Me |
| 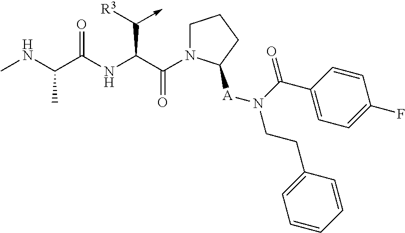 | 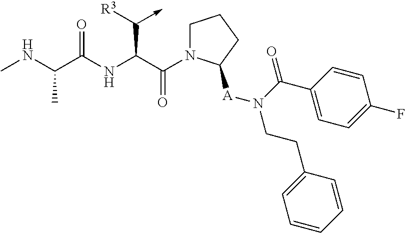 |
| A = CH₂; R³ = H or Me | A = CH₂; R³ = H or Me |
| 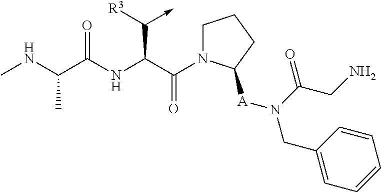 | 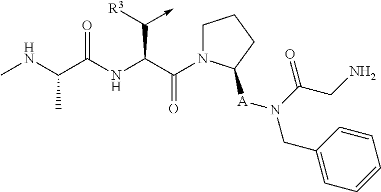 |
| A = CH₂; R³ = H or Me | A = CH₂; R³ = H or Me |
| 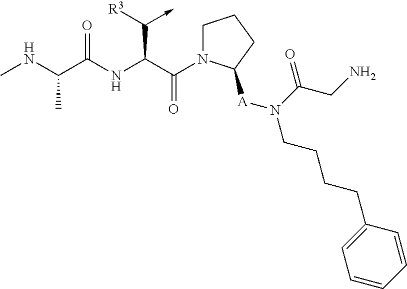 | 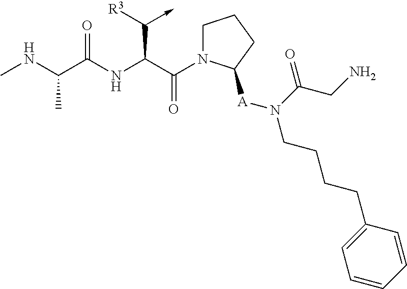 |
| A = CH₂; R³ = H or Me | A = CH₂; R³ = H or Me |
| 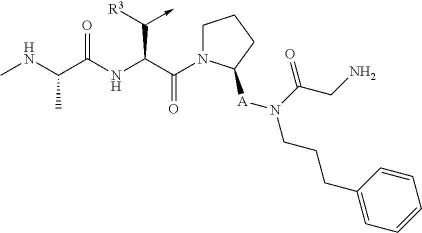 | 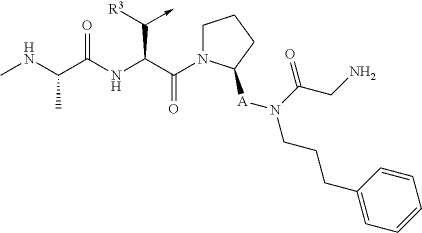 |
| A = CH₂; R³ = H or Me | A = CH₂; R³ = H or Me |
| 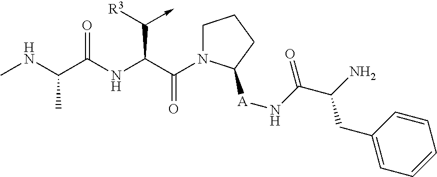 | 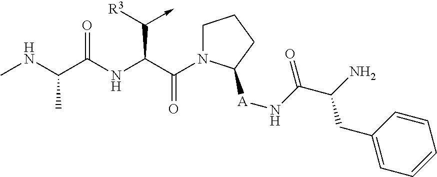 |
| A = CH₂; R³ = H or Me | A = CH₂; R³ = H or Me |
| 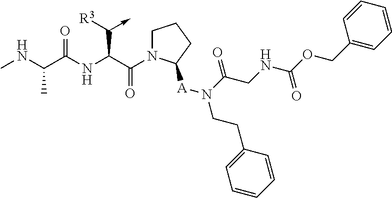 | 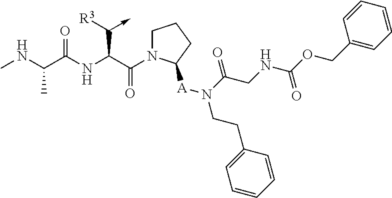 |
| A = CH₂; R³ = H or Me | A = CH₂; R³ = H or Me |
| 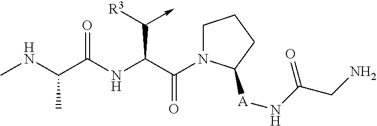 | 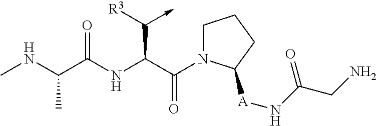 |
| A = CH₂; R³ = H or Me | A = CH₂; R³ = H or Me |
| 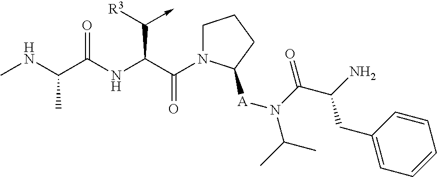 | 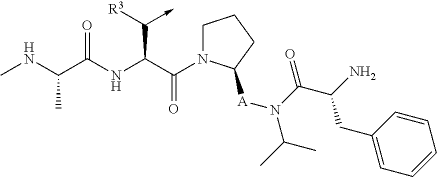 |
| 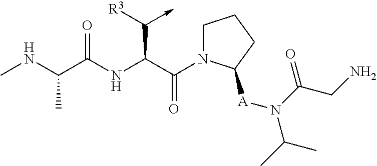 | 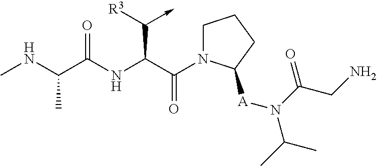 |
| A = CH₂; R³ = H or Me | A = CH₂; R³ = H or Me |
| 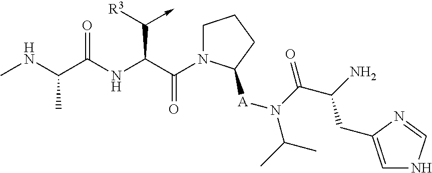 | 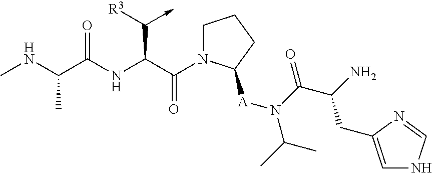 |
| A = CH₂; R³ = H or Me | A = CH₂; R³ = H or Me |
| 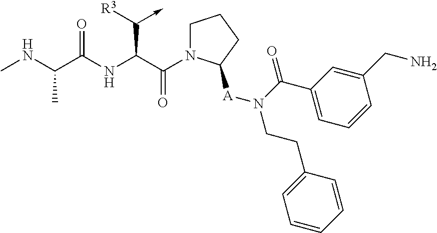 | 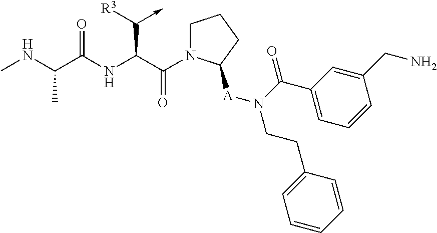 |
| A = CH₂; R³ = H or Me | A = CH₂; R³ = H or Me |
| 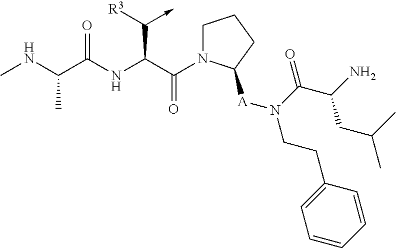 | 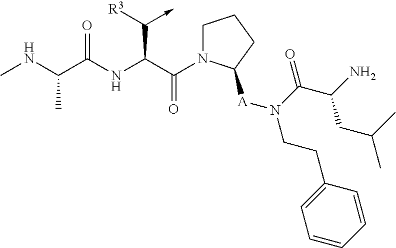 |
| A = CH₂; R³ = H or Me | A = CH₂; R³ = H or Me |
| 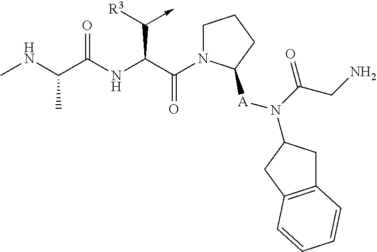 | 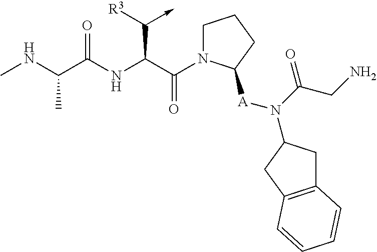 |
| A = CH₂; R³ = H or Me | A = CH₂; R³ = H or Me |
| 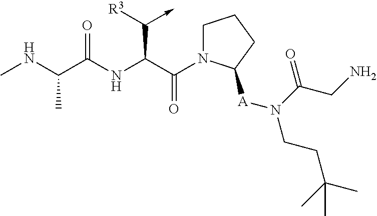 | 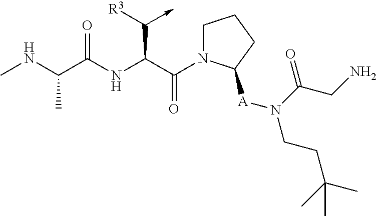 |
| A = CH₂; R³ = H or Me | A = CH₂; R³ = H or Me |
| 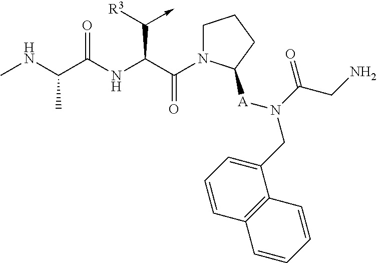 | 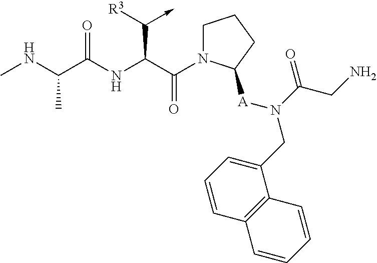 |
| A = CH₂; R³ = H or Me | A = CH₂; R³ = H or Me |
| 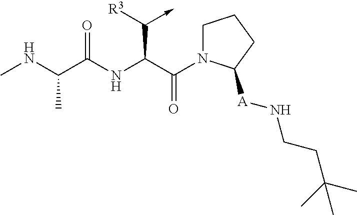 | 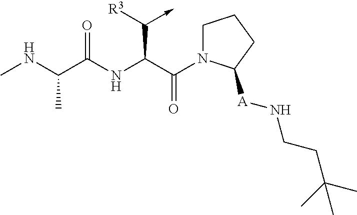 |
| A = CH₂; R³ = H or Me | A = CH₂; R³ = H or Me |
| 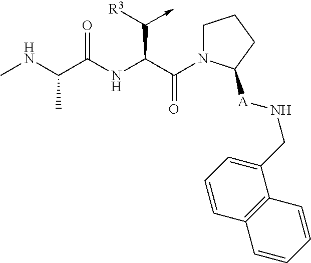 | 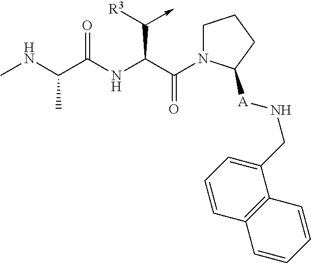 |
| A = CH₂; R³ = H or Me | A = CH₂; R³ = H or Me |
| 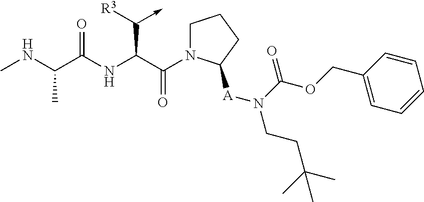 | 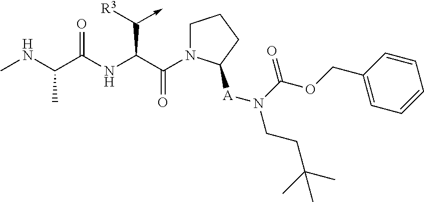 |
| A = CH₂; R³ = H or Me | A = CH₂; R³ = H or Me |
| 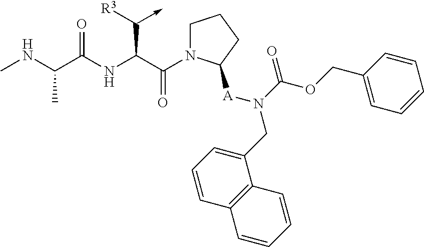 | 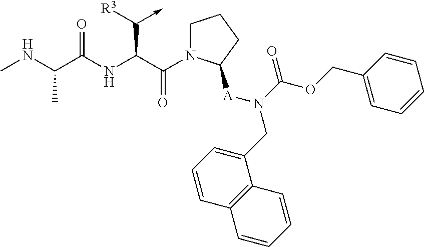 |
| A = CH₂; R³ = H or Me | A = CH₂; R³ = H or Me |
| 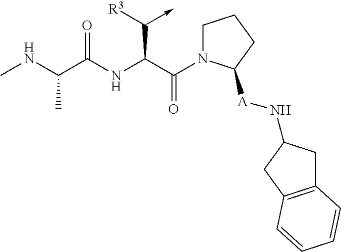 | 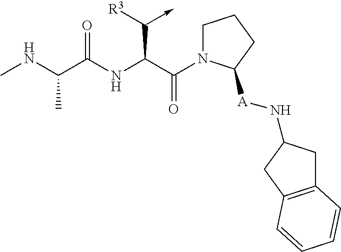 |
| A = CH₂; R³ = H or Me | A = CH₂; R³ = H or Me |
| 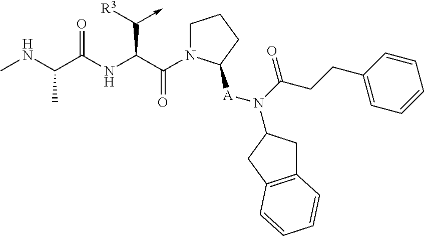 | 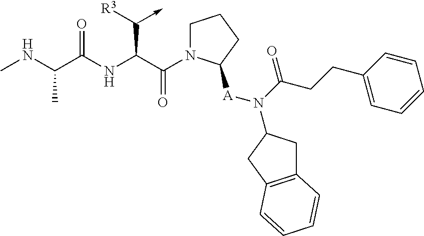 |
| A = CH₂; R³ = H or Me | A = CH₂; R³ = H or Me |
| 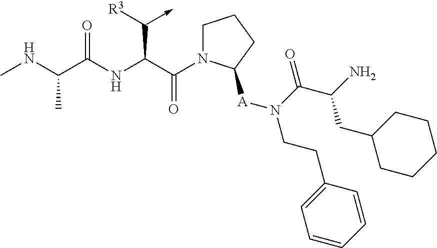 | 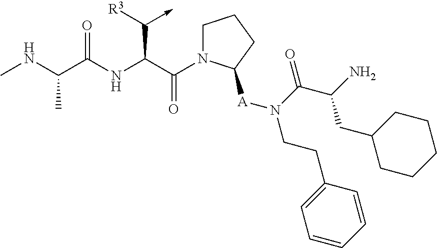 |
| A = CH₂; R³ = H or Me | A = CH₂; R³ = H or Me |
| 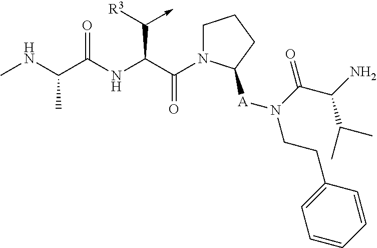 | 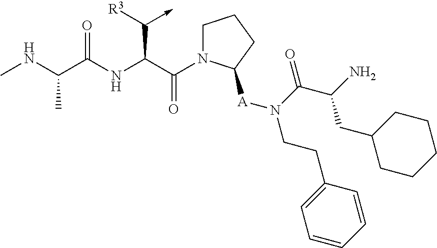 |
| A = CH₂; R³ = H or Me | A = CH₂; R³ = H or Me |
| 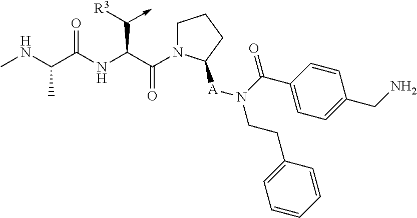 | 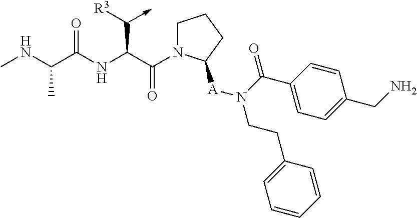 |
| A = CH₂; R³ = H or Me | A = CH₂; R³ = H or Me |
| 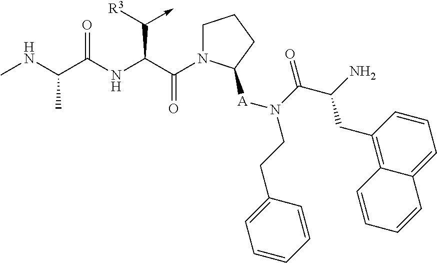 | 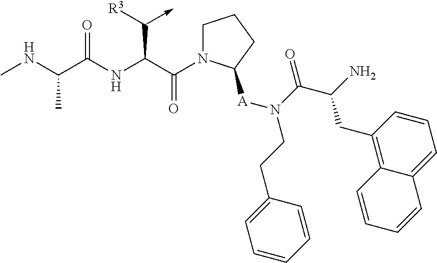 |
| A = CH₂; R³ = H or Me | A = CH₂; R³ = H or Me |
| 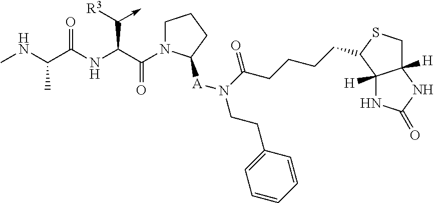 | 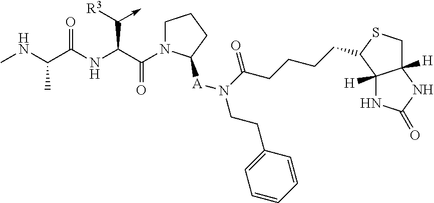 |
| A = CH₂; R³ = H or Me | A = CH₂; R³ = H or Me |
| 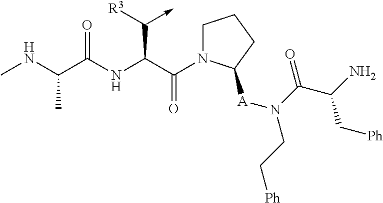 | 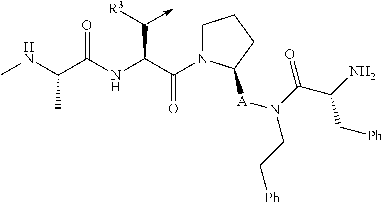 |
| A = CH₂; R³ = H or Me | A = CH₂; R³ = H or Me |
| 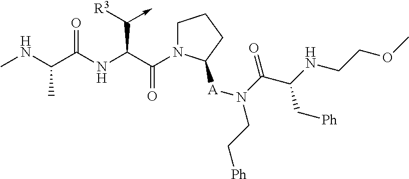 | 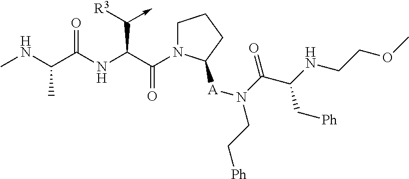 |
| A = CH₂; R³ = H or Me | A = CH₂; R³ = H or Me |
| 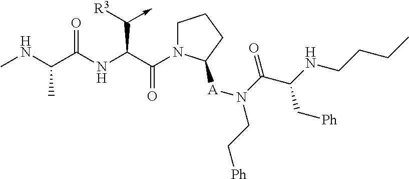 | 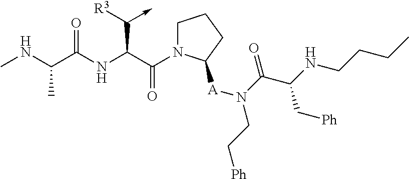 |
| 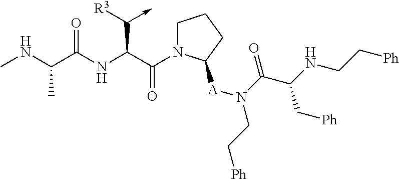 | 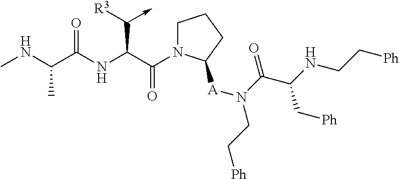 |
| A = CH₂; R³ = H or Me | A = CH₂; R³ = H or Me |
| 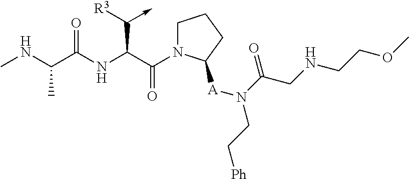 | 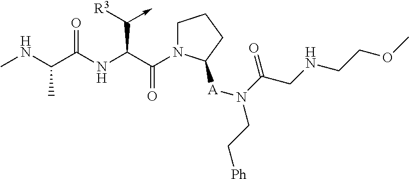 |
| A = CH₂; R³ = H or Me | A = CH₂; R³ = H or Me |
| 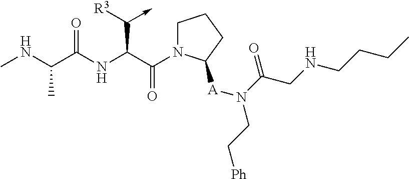 | 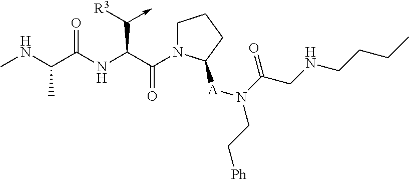 |
| A = CH₂; R³ = H or Me | A = CH₂; R³ = H or Me |
| 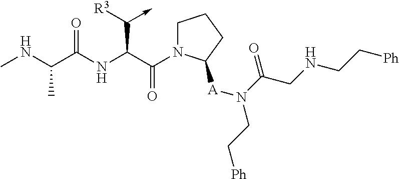 | 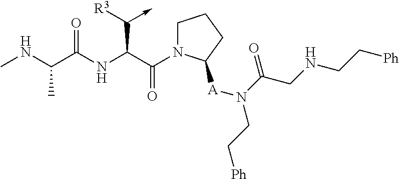 |
| A = CH₂; R³ = H or Me | A = CH₂; R³ = H or Me |
| 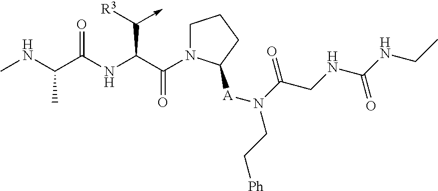 | 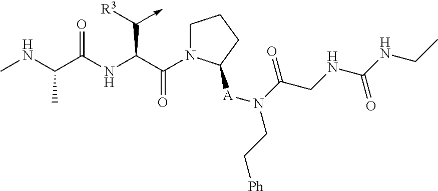 |
| A = CH₂; R³ = H or Me | A = CH₂; R³ = H or Me |
| 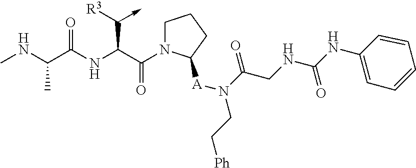 | 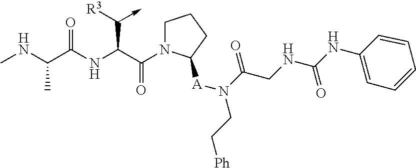 |
| A = CH₂; R³ = H or Me | A = CH₂; R³ = H or Me |
| 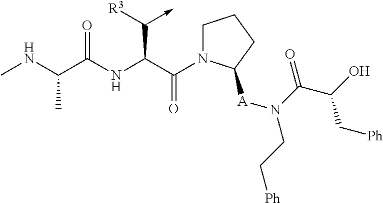 | 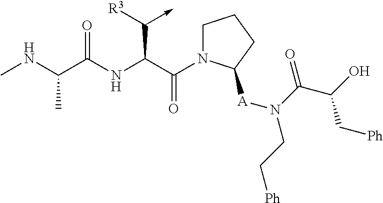 |
| A = CH₂; R³ = H or Me | A = CH₂; R³ = H or Me |
| 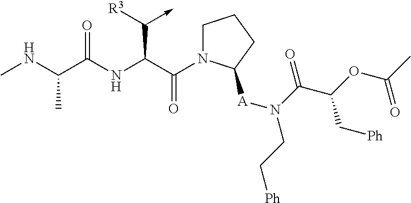 | 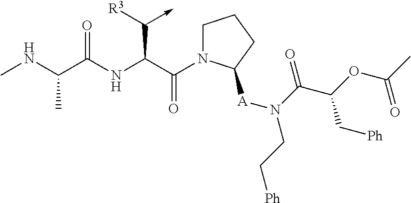 |
| A = CH₂; R³ = H or Me | A = CH₂; R³ = H or Me |
| 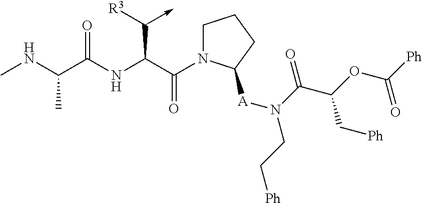 | 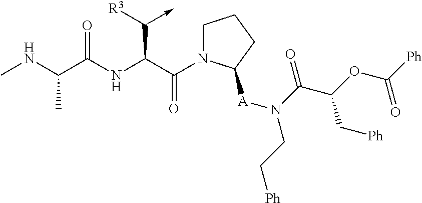 |
| A = CH₂; R³ = H or Me | A = CH₂; R³ = H or Me |
| 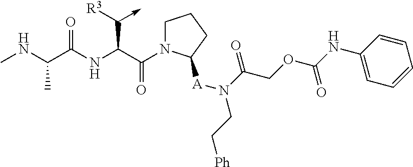 | 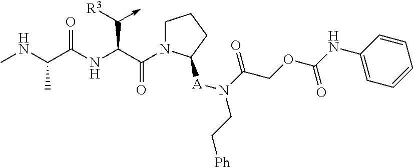 |
| A = CH₂; R³ = H or Me | A = CH₂; R³ = H or Me |
| 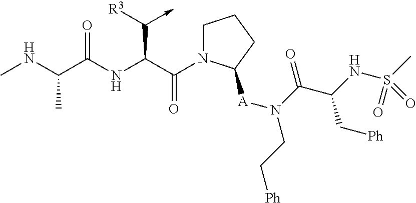 | 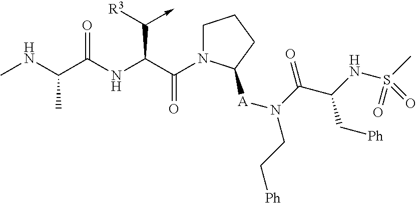 |
| A = CH₂; R³ = H or Me | A = CH₂; R³ = H or Me |
| 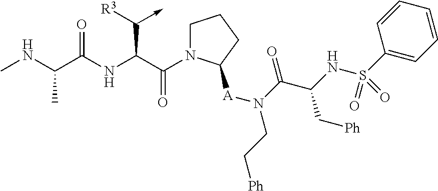 | 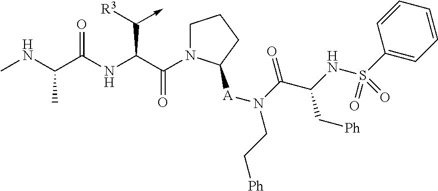 |
| A = CH₂; R³ = H or Me | A = CH₂; R³ = H or Me |
| 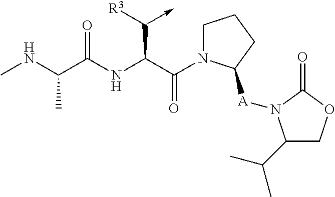 | 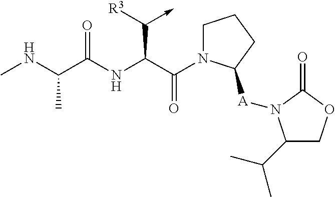 |
| A = CH₂; R³ = H or Me | A = CH₂; R³ = H or Me |
| 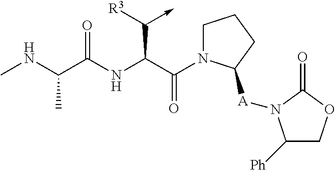 | 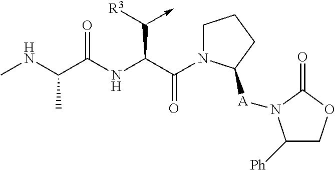 |
| A = CH₂; R³ = H or Me | A = CH₂; R³ = H or Me |
| 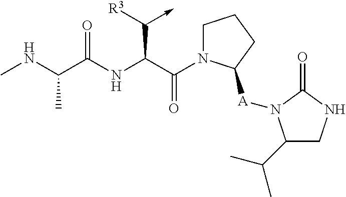 | 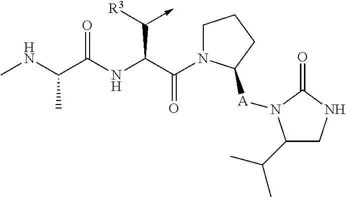 |
| A = CH₂; R³ = H or Me | A = CH₂; R³ = H or Me |
| 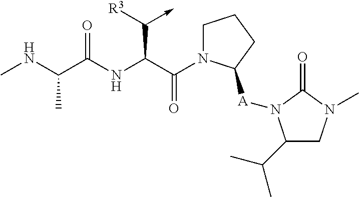 | 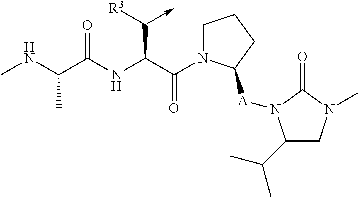 |
| A = CH₂; R³ = H or Me | A = CH₂; R³ = H or Me |
| 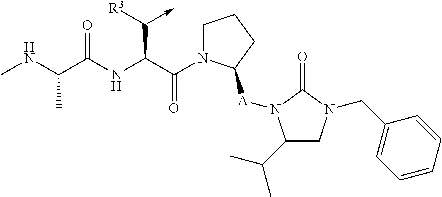 | 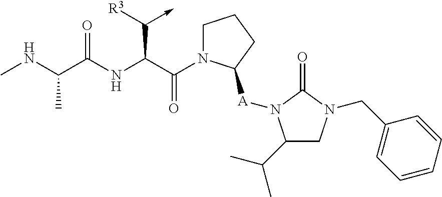 |
| A = CH₂; R³ = H or Me | A = CH₂; R³ = H or Me |
| 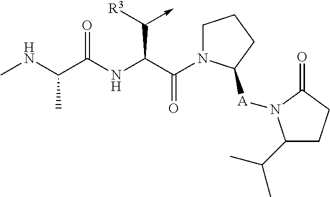 | 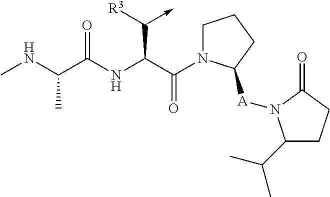 |
| A = CH₂; R³ = H or Me | A = CH₂; R³ = H or Me |
| 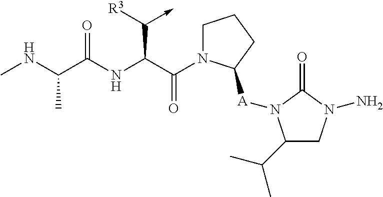 | 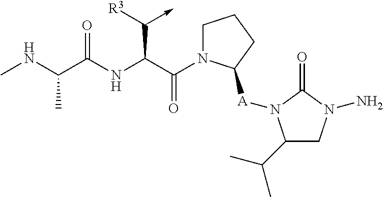 |
| A = CH₂; R³ = H or Me | A = CH₂; R³ = H or Me |
| 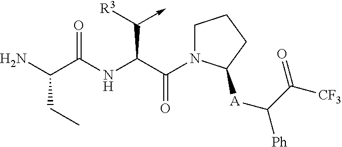 | 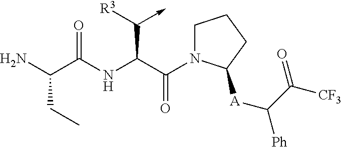 |
| A = CH₂; R³ = H or Me | A = CH₂; R³ = H or Me |
| 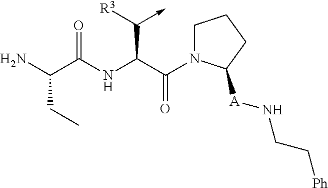 | 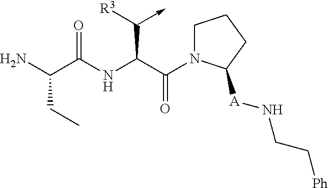 |
| A = CH₂; R³ = H or Me | A = CH₂; R³ = H or Me |
| 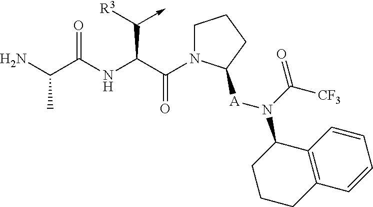 | 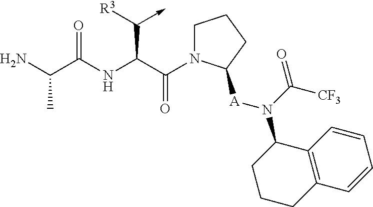 |
| A = CH₂; R³ = H or Me | A = CH₂; R³ = H or Me |
| 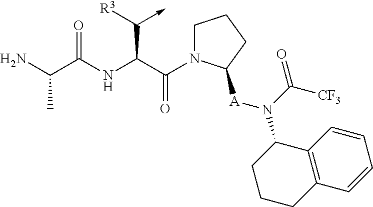 | 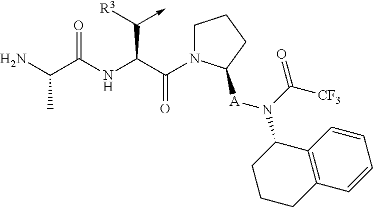 |
| A = CH₂; R³ = H or Me | A = CH₂; R³ = H or Me |
| 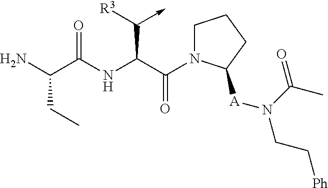 | 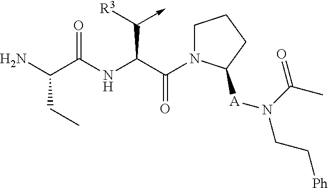 |
| A = CH₂; R³ = H or Me | A = CH₂; R³ = H or Me |
| 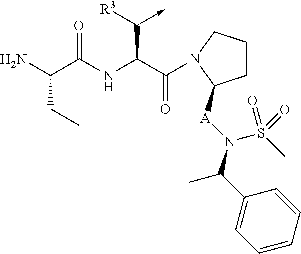 | 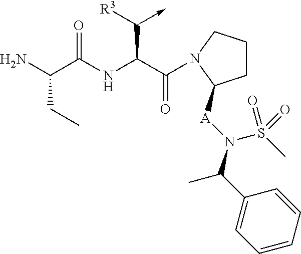 |
| A = CH₂; R³ = H or Me | A = CH₂; R³ = H or Me |
| 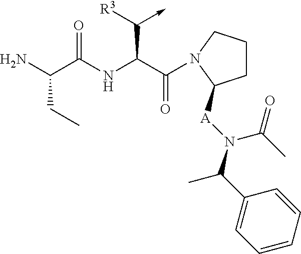 | 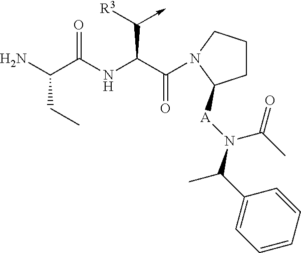 |
| A = CH₂; R³ = H or Me | A = CH₂; R³ = H or Me |
| 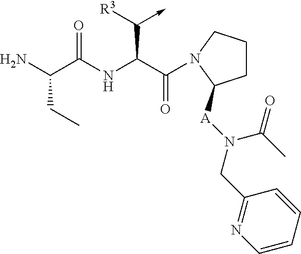 | 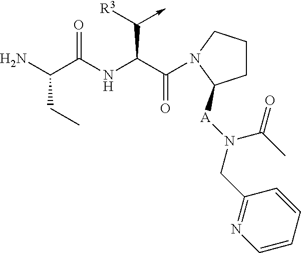 |
| A = CH₂; R³ = H or Me | A = CH₂; R³ = H or Me |
| 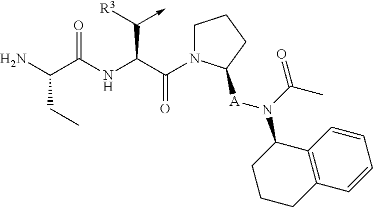 | 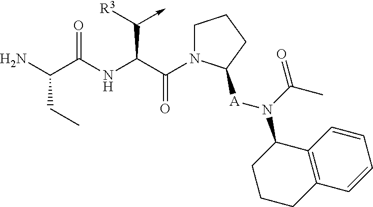 |
| A = CH₂; R³ = H or Me | A = CH₂; R³ = H or Me |
| 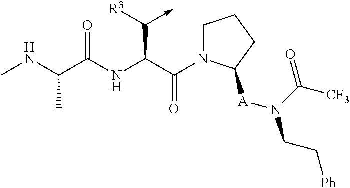 | 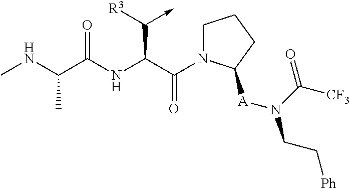 |
| A = CH₂; R³ = H or Me | A = CH₂; R³ = H or Me |
| 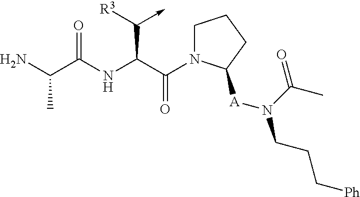 | 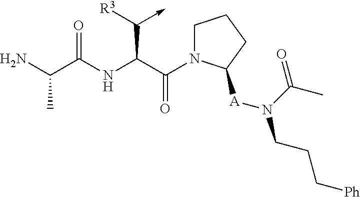 |
| A = CH₂; R³ = H or Me | A = CH₂; R³ = H or Me |
| 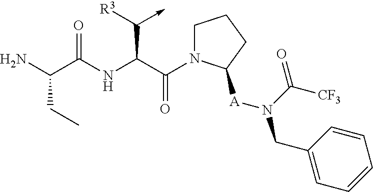 | 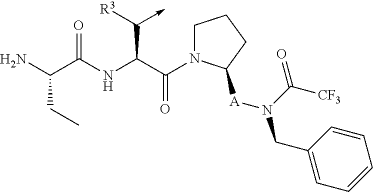 |
| A = CH₂; R³ = H or Me | A = CH₂; R³ = H or Me |
| 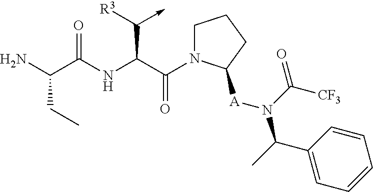 | 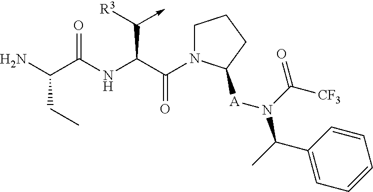 |
| A = CH₂; R³ = H or Me | A = CH₂; R³ = H or Me |
| 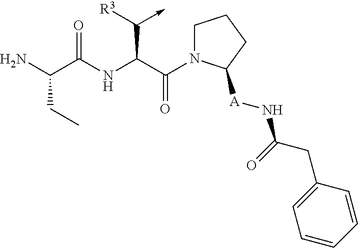 | 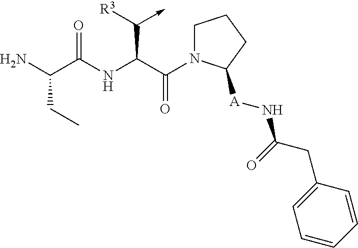 |
| A = CH₂; R³ = H or Me | A = CH₂; R³ = H or Me |
| 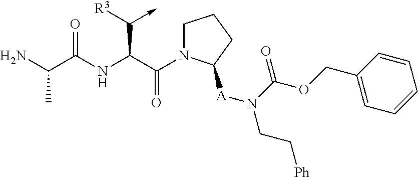 | 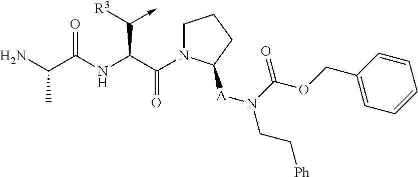 |
| A = CH₂; R³ = H or Me | A = CH₂; R³ = H or Me |
| 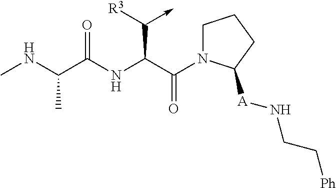 | 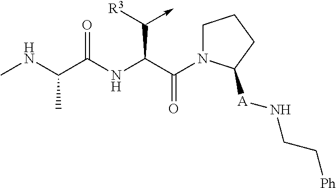 |
| A = CH₂; R³ = H or Me | A = CH₂; R³ = H or Me |
| 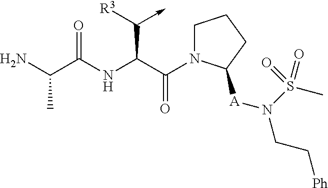 | 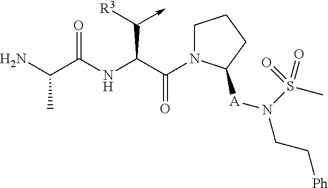 |
| A = CH₂; R³ = H or Me | A = CH₂; R³ = H or Me |
| 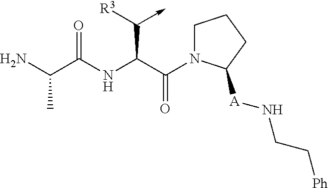 | 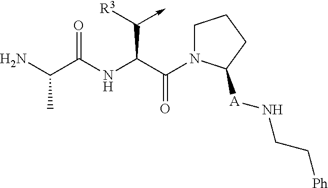 |
| A = CH₂; R³ = H or Me | A = CH₂; R³ = H or Me |
| 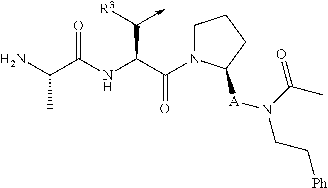 | 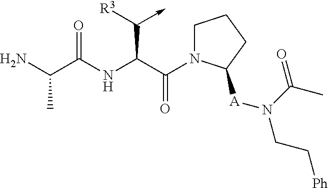 |
| A = CH₂; R³ = H or Me | A = CH₂; R³ = H or Me |
| 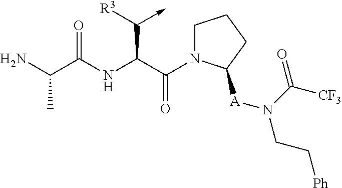 | 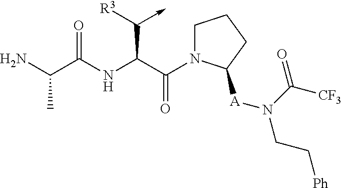 |
| A = CH₂; R³ = H or Me | A = CH₂; R³ = H or Me |
| 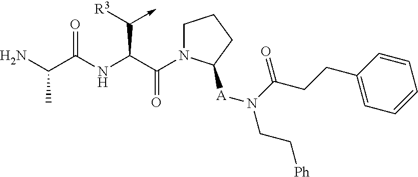 | 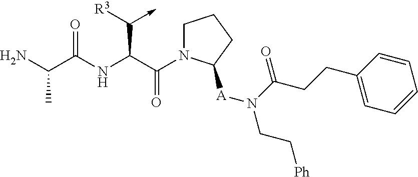 |
| A = CH₂; R³ = H or Me | A = CH₂; R³ = H or Me |
| 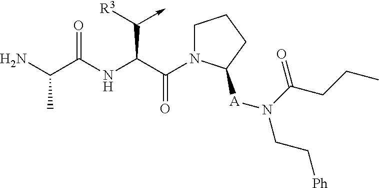 | 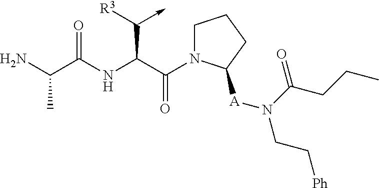 |
| A = CH₂; R³ = H or Me | A = CH₂; R³ = H or Me |
| 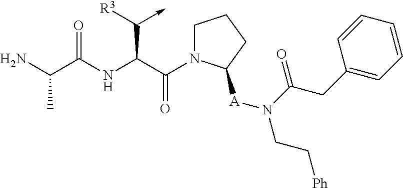 | 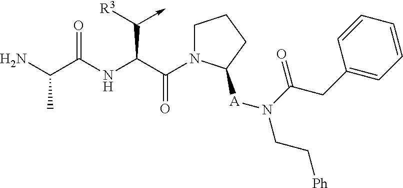 |
| A = CH₂; R³ = H or Me | A = CH₂; R³ = H or Me |
| 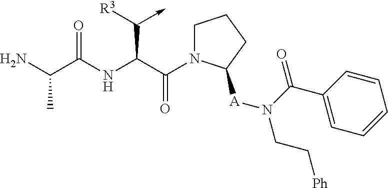 | 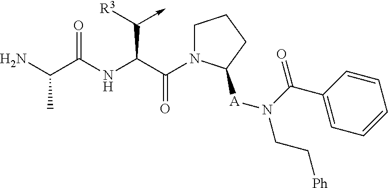 |
| A = CH₂; R³ = H or Me | A = CH₂; R³ = H or Me |
| 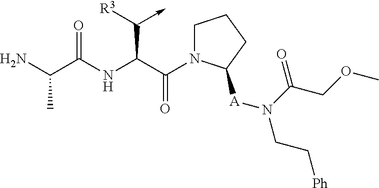 | 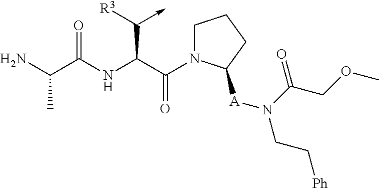 |
| A = CH₂; R³ = H or Me | A = CH₂; R³ = H or Me |
| 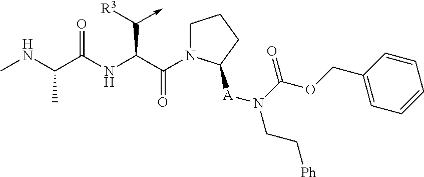 | 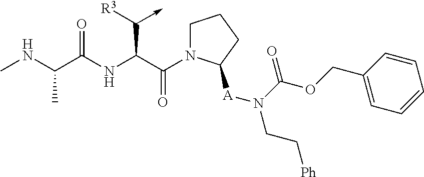 |
| A = CH₂; R³ = H or Me | A = CH₂; R³ = H or Me |
| 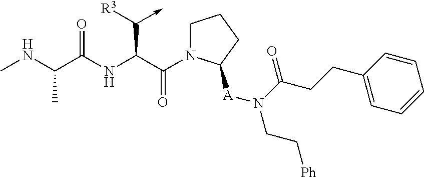 | 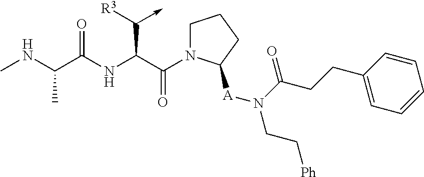 |
| A = CH₂; R³ = H or Me | A = CH₂; R³ = H or Me |
| 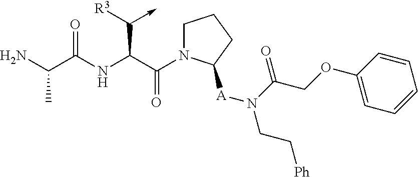 | 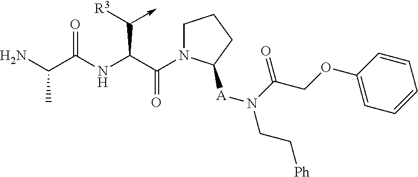 |
| A = CH₂; R³ = H or Me | A = CH₂; R³ = H or Me |
| 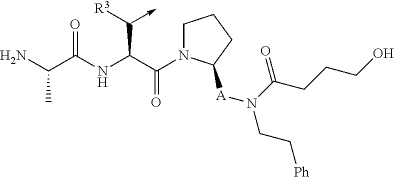 | 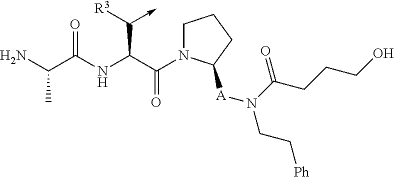 |
| A = CH₂; R³ = H or Me | A = CH₂; R³ = H or Me |
| 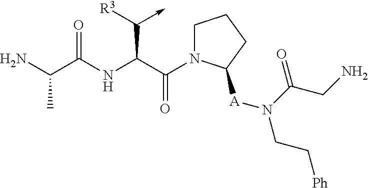 | 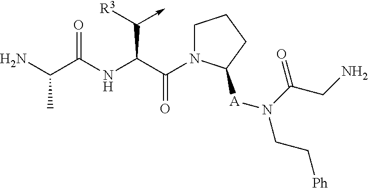 |
| A = CH₂; R³ = H or Me | A = CH₂; R³ = H or Me |
| 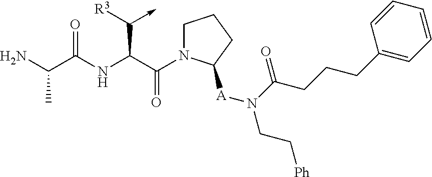 | 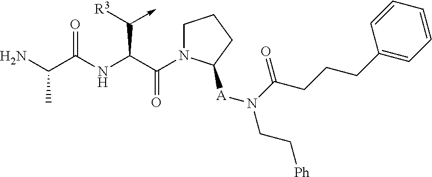 |
| A = CH₂; R³ = H or Me | A = CH₂; R³ = H or Me |
| 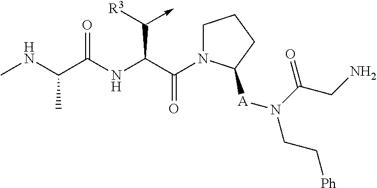 | 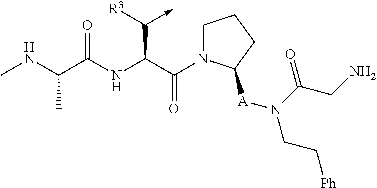 |
| A = CH₂; R³ = H or Me | A = CH₂; R³ = H or Me |
| 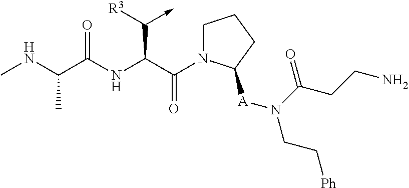 | 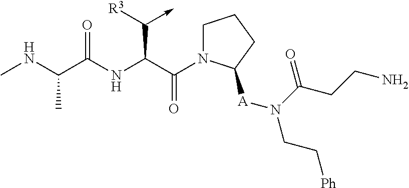 |
| A = CH₂; R³ = H or Me | A = CH₂; R³ = H or Me |
| 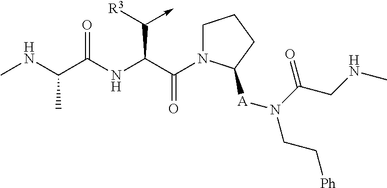 | 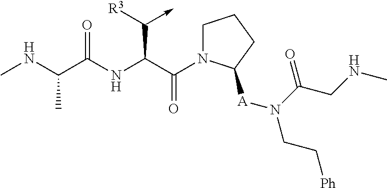 |
| A = CH₂; R³ = H or Me | A = CH₂; R³ = H or Me |
| 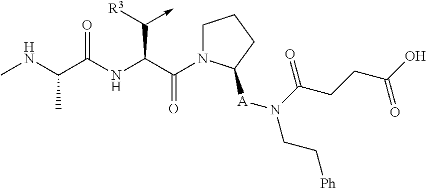 | 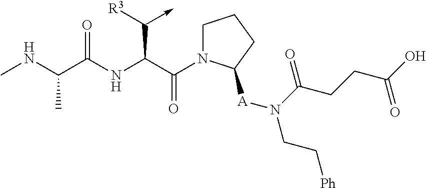 |
| A = CH₂; R³ = H or Me | A = CH₂; R³ = H or Me |
| 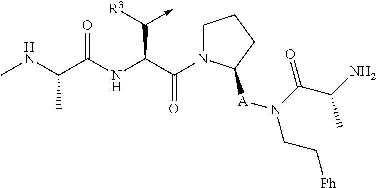 | 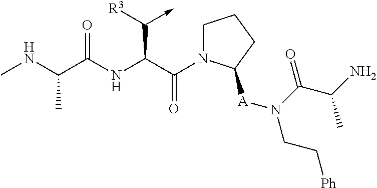 |
| A = CH₂; R³ = H or Me | A = CH₂; R³ = H or Me |
| 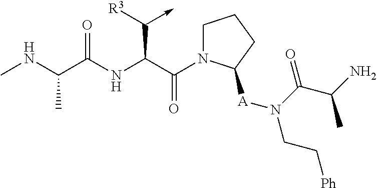 | 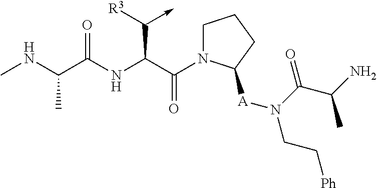 |
| A = CH₂; R³ = H or Me | A = CH₂; R³ = H or Me |
| 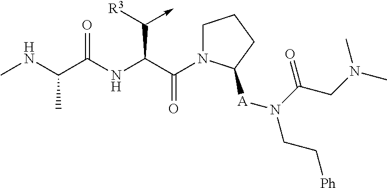 | 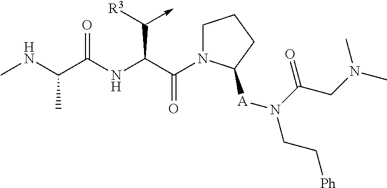 |
| A = CH₂; R³ = H or Me | A = CH₂; R³ = H or Me |
| 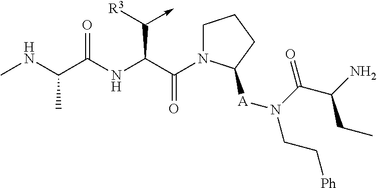 | 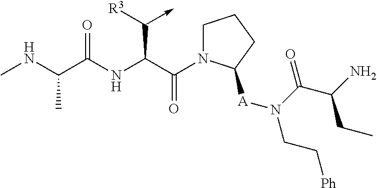 |
| A = CH₂; R³ = H or Me | A = CH₂; R³ = H or Me |
| 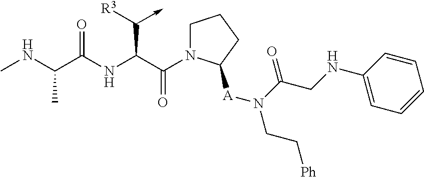 | 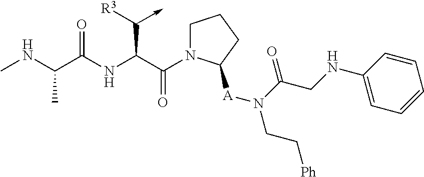 |
| A = CH₂; R³ = H or Me | A = CH₂; R³ = H or Me |
| 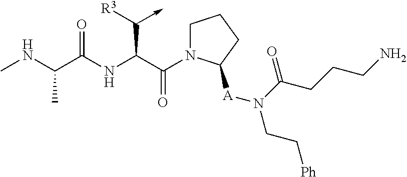 | 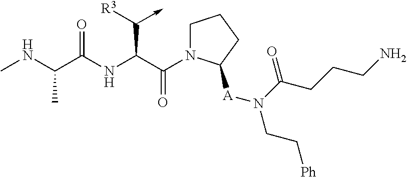 |
| A = CH₂; R³ = H or Me | A = CH₂; R³ = H or Me |
| 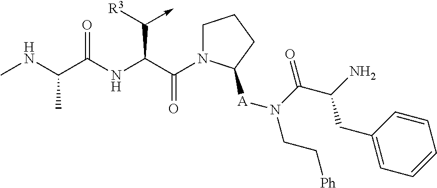 | 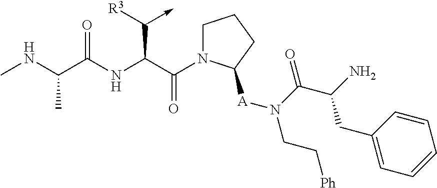 |
| A = CH₂; R³ = H or Me | A = CH₂; R³ = H or Me |
| 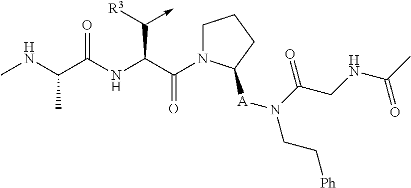 | 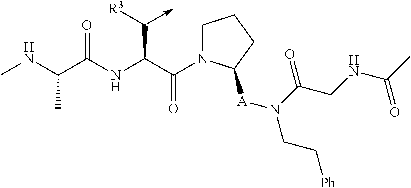 |
| A = CH₂; R³ = H or Me | A = CH₂; R³ = H or Me |
| 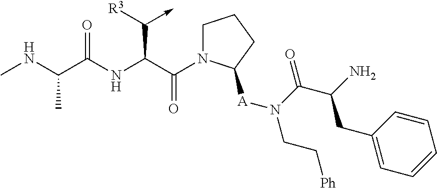 | 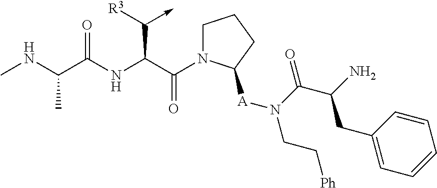 |
| A = CH₂; R³ = H or Me | A = CH₂; R³ = H or Me |
| 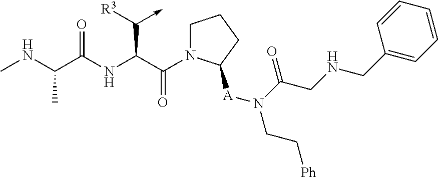 | 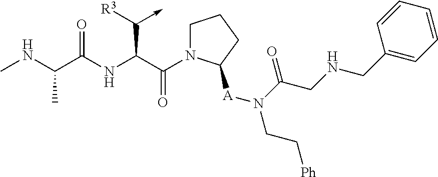 |
| A = CH₂; R³ = H or Me | A = CH₂; R³ = H or Me |
| 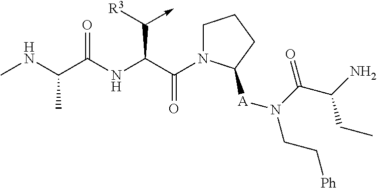 | 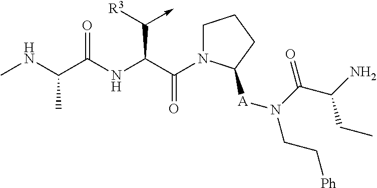 |
| A = CH₂; R³ = H or Me | A = CH₂; R³ = H or Me |
| 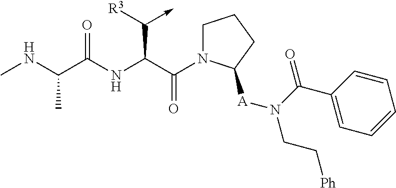 | 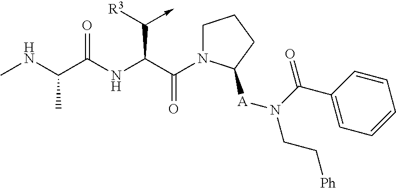 |
| A = CH₂; R³ = H or Me | A = CH₂; R³ = H or Me |
| 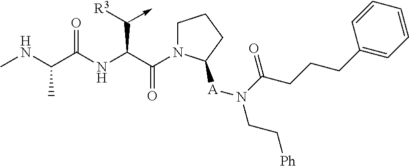 | 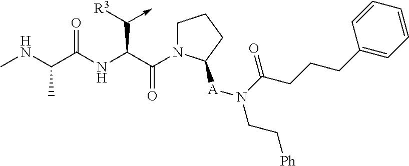 |
| A = CH₂; R³ = H or Me | A = CH₂; R³ = H or Me |
| 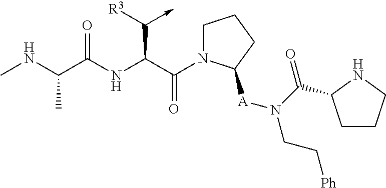 | 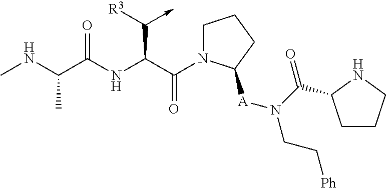 |
| A = CH₂; R³ = H or Me | A = CH₂; R³ = H or Me |
| 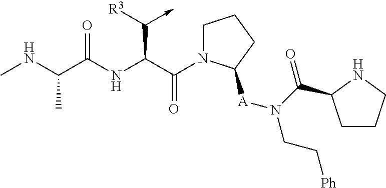 | 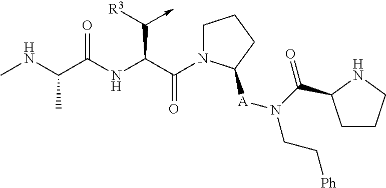 |
| A = CH₂; R³ = H or Me | A = CH₂; R³ = H or Me |
| 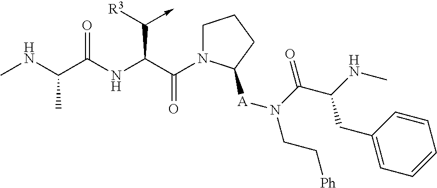 | 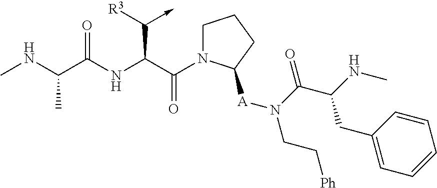 |
| A = CH₂; R³ = H or Me | A = CH₂; R³ = H or Me |
| 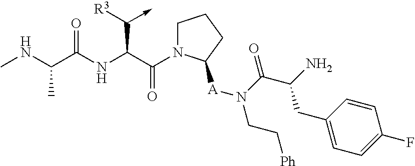 | 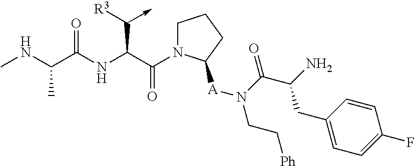 |
| A = CH₂; R³ = H or Me | A = CH₂; R³ = H or Me |
| 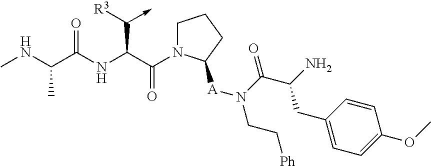 | 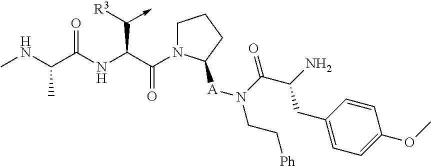 |
| A = CH₂; R³ = H or Me | A = CH₂; R³ = H or Me |
| 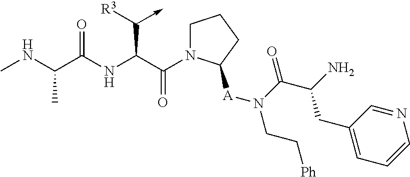 | 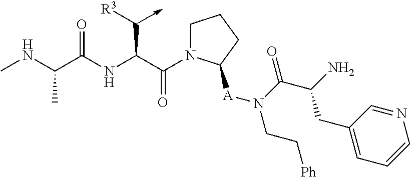 |
| A = CH₂; R³ = H or Me | A = CH₂; R³ = H or Me |
| 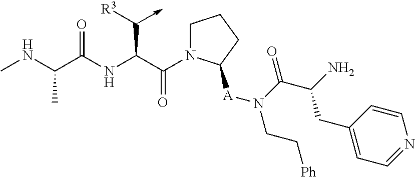 | 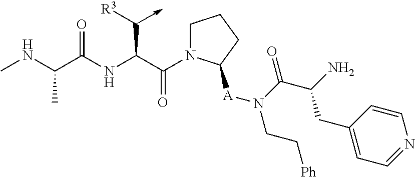 |
| A = CH₂; R³ = H or Me | A = CH₂; R³ = H or Me |
| 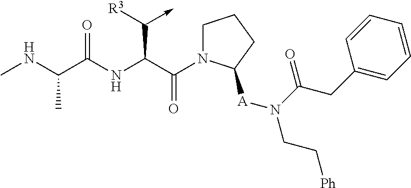 | 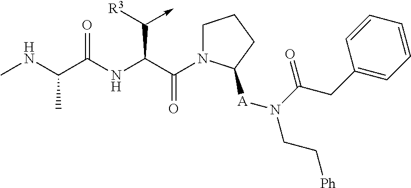 |
| A = CH₂; R³ = H or Me | A = CH₂; R³ = H or Me |
| 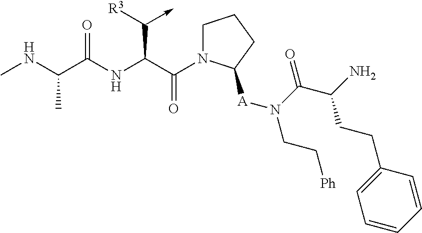 | 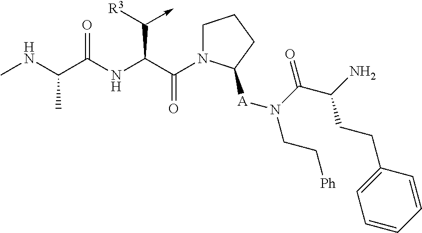 |
| A = CH₂; R³ = H or Me | A = CH₂; R³ = H or Me |
| 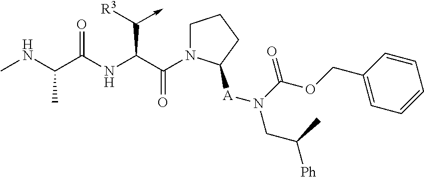 | 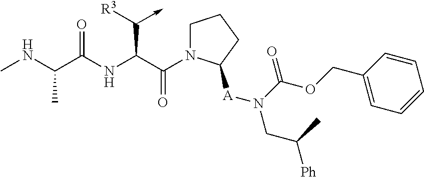 |
| A = CH₂; R³ = H or Me | A = CH₂; R³ = H or Me |
| 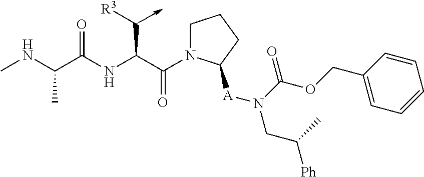 | 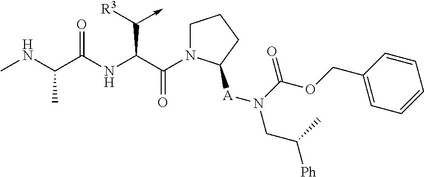 |
| A = CH₂; R³ = H or Me | A = CH₂; R³ = H or Me |
| 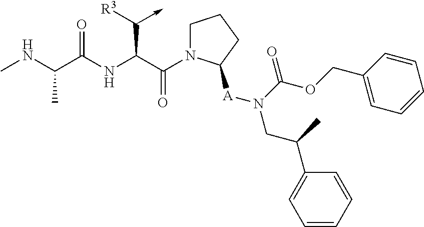 | 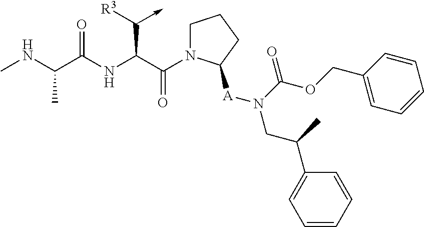 |
| A = CH₂; R³ = H or Me | A = CH₂; R³ = H or Me |
| 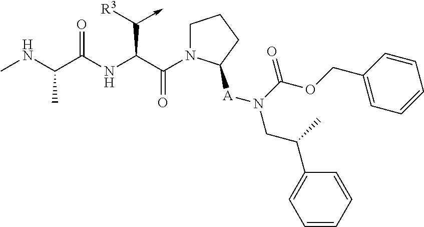 | 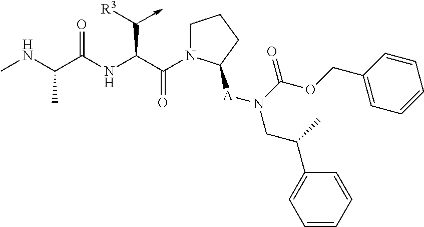 |
| A = CH₂; R³ = H or Me | A = CH₂; R³ = H or Me |
| 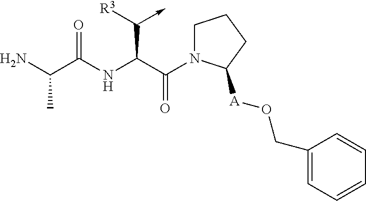 | 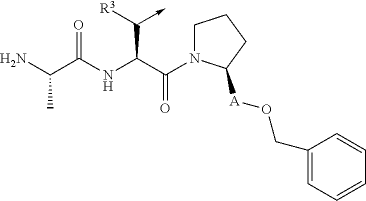 |
| A = CH₂; R³ = H or Me | A = CH₂; R³ = H or Me |
| 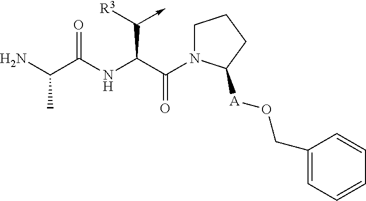 | 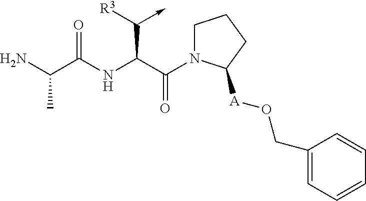 |
| A = CH₂; R³ = H or Me | A = CH₂; R³ = H or Me |
| 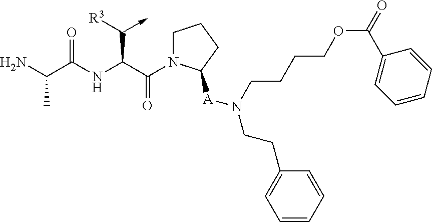 | 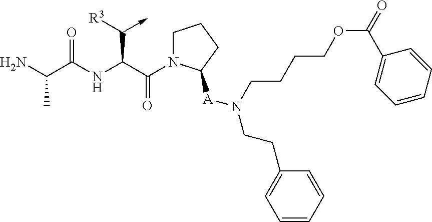 |
| A = CH₂; R³ = H or Me | A = CH₂; R³ = H or Me |
| 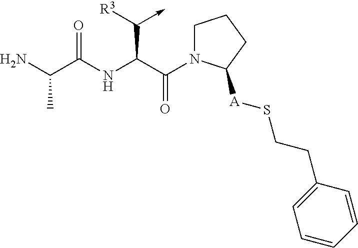 | 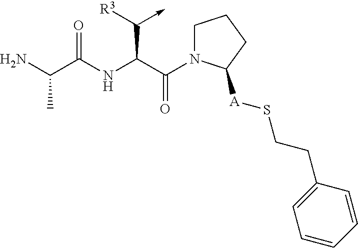 |
| A = CH₂; R³ = H or Me | A = CH₂; R³ = H or Me |
| 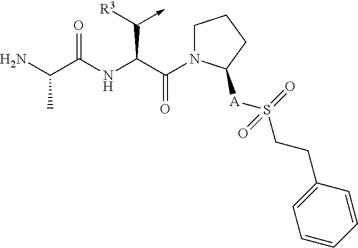 | 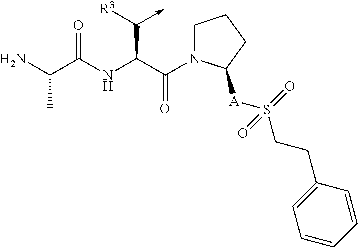 |
| A = CH₂; R³ = H or Me | A = CH₂; R³ = H or Me |
| 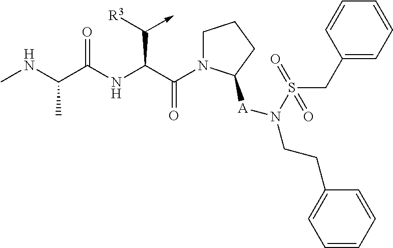 | 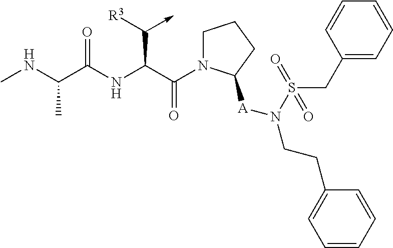 |
| A = CH₂; R³ = H or Me | A = CH₂; R³ = H or Me |
| 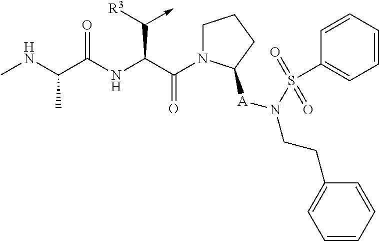 | 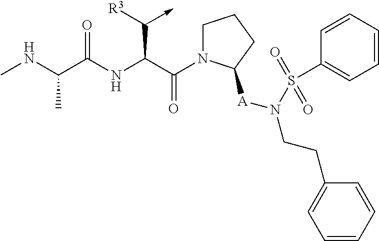 |
| A = CH₂; R³ = H or Me | A = CH₂; R³ = H or Me |
| 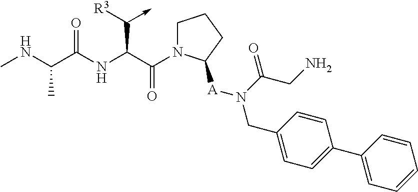 | 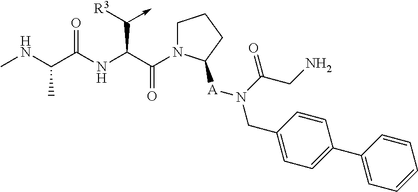 |
| A = CH₂; R³ = H or Me | A = CH₂; R³ = H or Me |
| 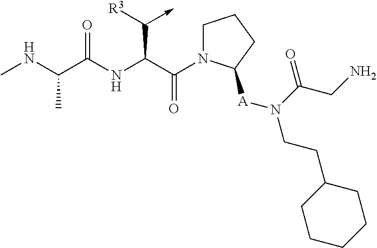 | 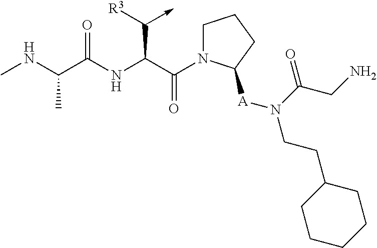 |
| A = CH₂; R³ = H or Me | A = CH₂; R³ = H or Me |
| 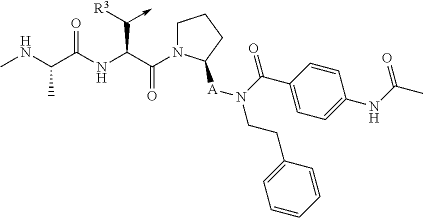 | 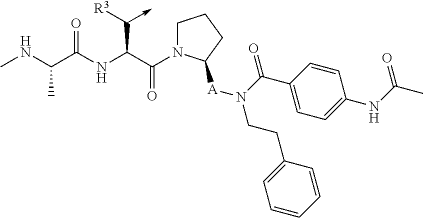 |
| A = CH₂; R³ = H or Me | A = CH₂; R³ = H or Me |
| 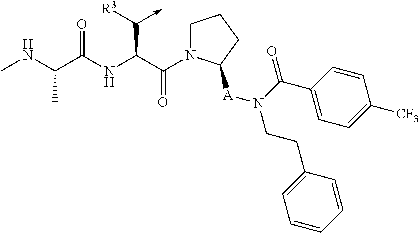 | 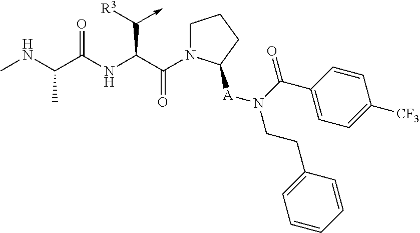 |
| A = CH₂; R³ = H or Me | A = CH₂; R³ = H or Me |
| 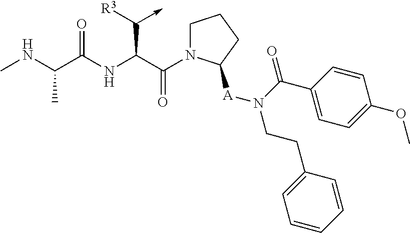 | 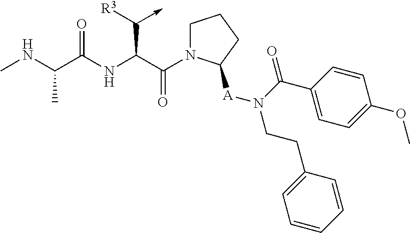 |
| A = CH₂; R³ = H or Me | A = CH₂; R³ = H or Me |
| 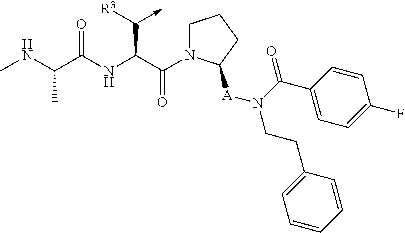 | 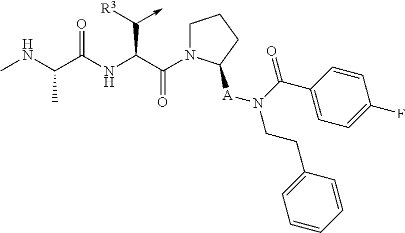 |
| A = CH₂; R³ = H or Me | A = CH₂; R³ = H or Me |
| 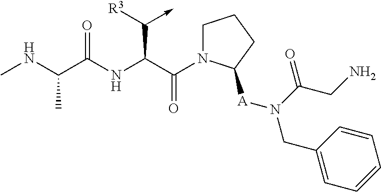 | 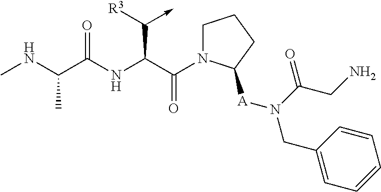 |
| A = CH₂; R³ = H or Me | A = CH₂; R³ = H or Me |
| 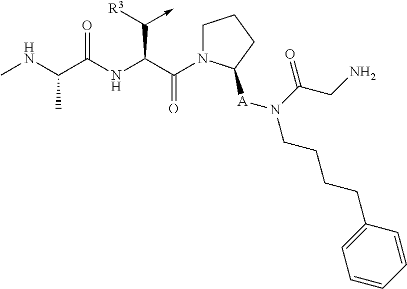 | 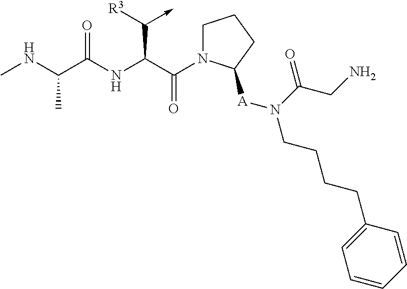 |
| A = CH₂; R³ = H or Me | A = CH₂; R³ = H or Me |
| 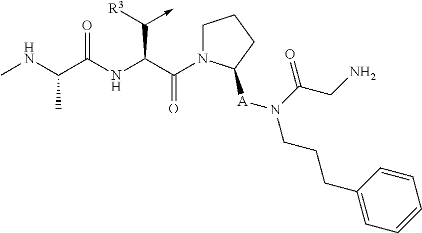 | 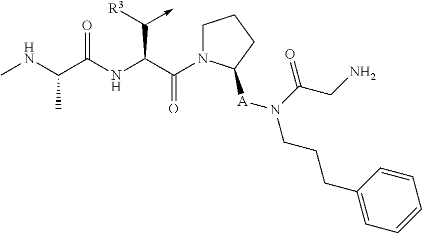 |
| A = CH₂; R³ = H or Me | A = CH₂; R³ = H or Me |
| 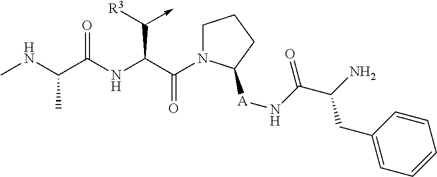 | 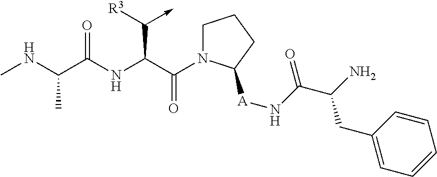 |
| A = CH₂; R³ = H or Me | A = CH₂; R³ = H or Me |
| 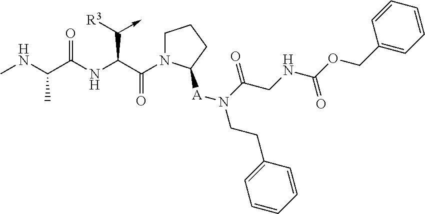 | 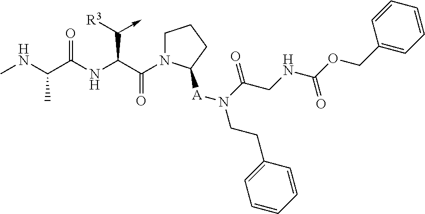 |
| A = CH₂; R³ = H or Me | A = CH₂; R³ = H or Me |
| 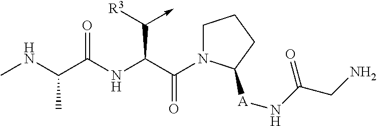 | 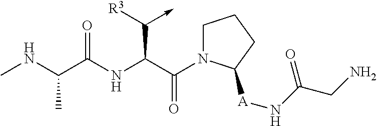 |
| A = CH₂; R³ = H or Me | A = CH₂; R³ = H or Me |
| 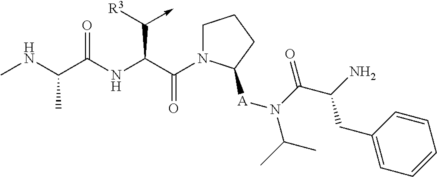 | 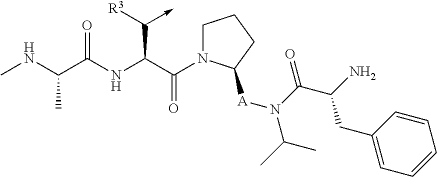 |
| A = CH₂; R³ = H or Me | A = CH₂; R³ = H or Me |
| 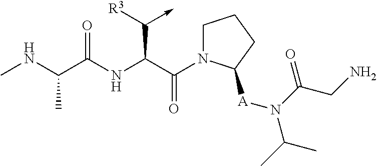 | 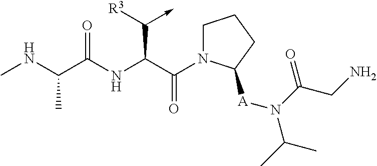 |
| A = CH₂; R³ = H or Me | A = CH₂; R³ = H or Me |
| 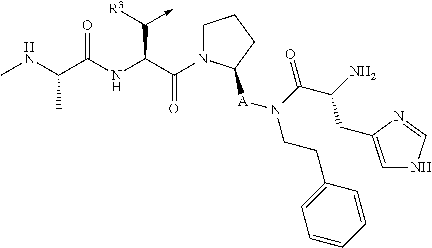 | 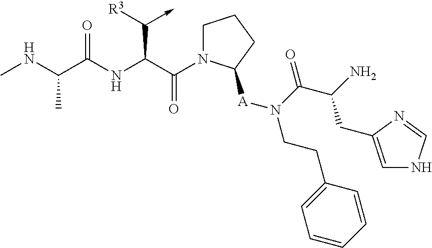 |
| A = CH₂; R³ = H or Me | A = CH₂; R³ = H or Me |
| 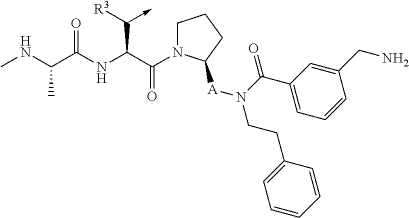 | 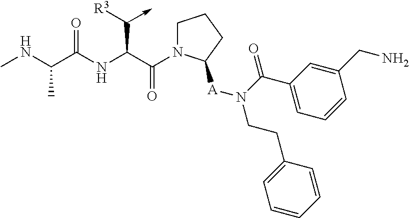 |
| A = CH₂; R³ = H or Me | A = CH₂; R³ = H or Me |
| 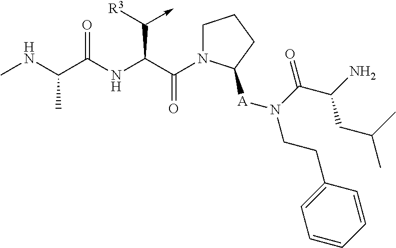 | 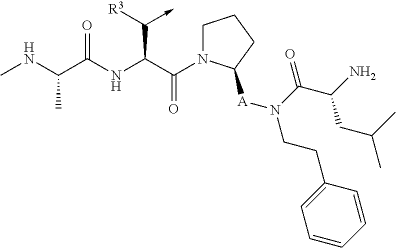 |
| A = CH₂; R³ = H or Me | A = CH₂; R³ = H or Me |
| 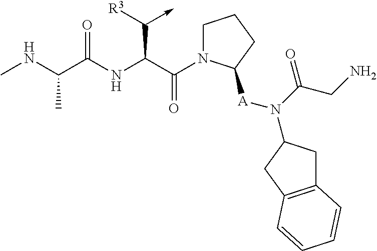 | 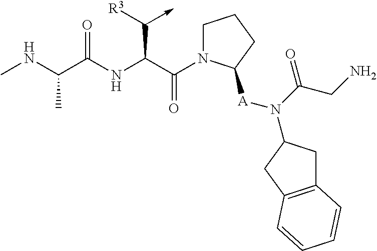 |
| A = CH₂; R³ = H or Me | A = CH₂; R³ = H or Me |
| 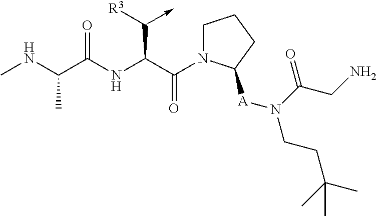 | 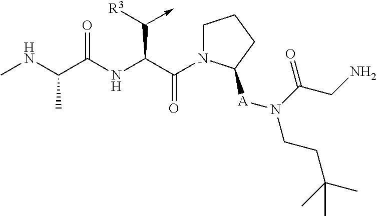 |
| A = CH₂; R³ = H or Me | A = CH₂; R³ = H or Me |
| 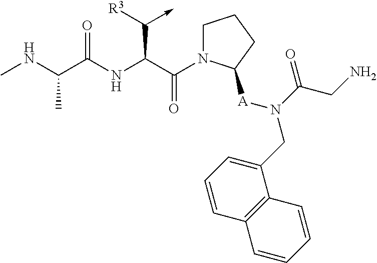 | 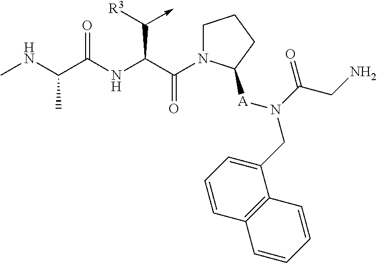 |
| A = CH₂; R³ = H or Me | A = CH₂; R³ = H or Me |
| 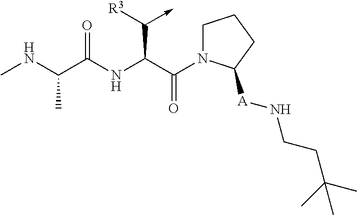 | 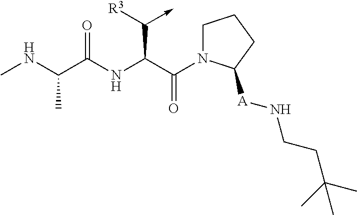 |
| A = CH₂; R³ = H or Me | A = CH₂; R³ = H or Me |
| 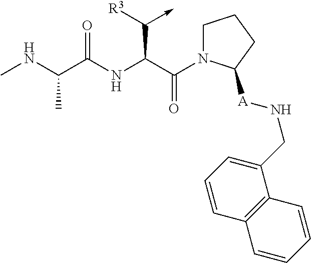 | 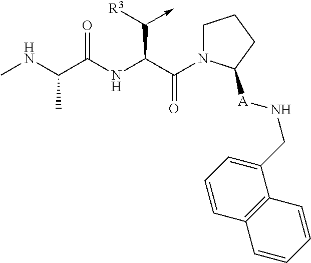 |
| A = CH₂; R³ = H or Me | A = CH₂; R³ = H or Me |
| 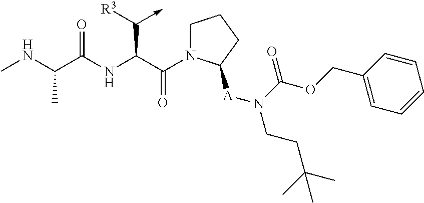 | 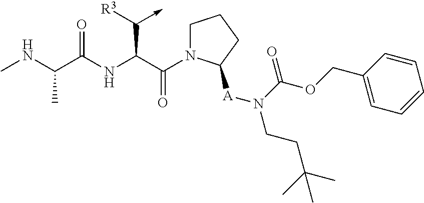 |
| A = CH₂; R³ = H or Me | A = CH₂; R³ = H or Me |
| 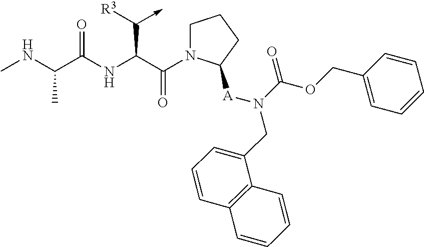 | 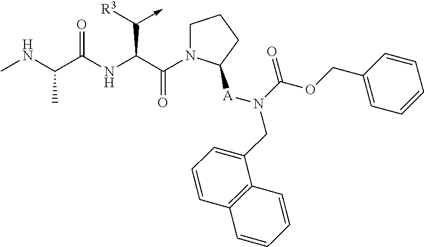 |
| A = CH₂; R³ = H or Me | A = CH₂; R³ = H or Me |
| 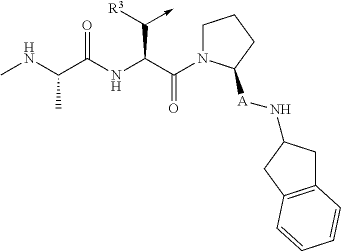 | 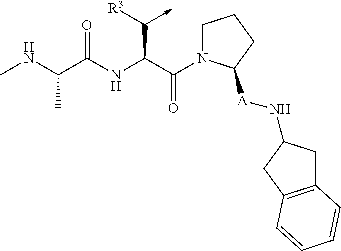 |
| A = CH₂; R³ = H or Me | A = CH₂; R³ = H or Me |
| 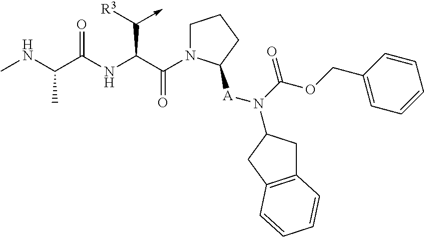 | 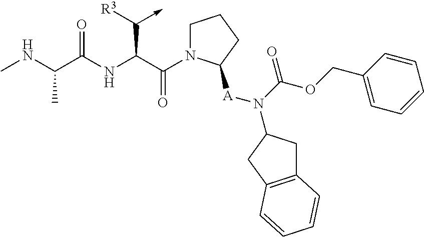 |
| A = CH₂; R³ = H or Me | A = CH₂; R³ = H or Me |
| 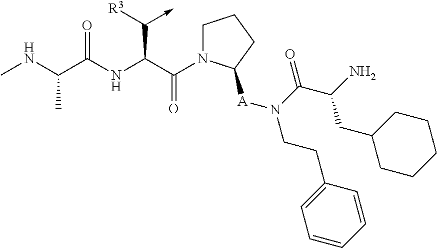 | 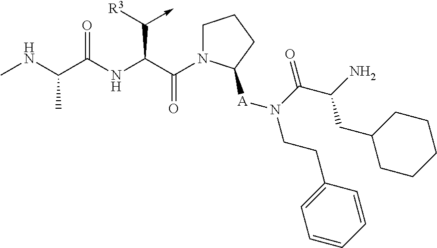 |
| A = CH₂; R³ = H or Me | A = CH₂; R³ = H or Me |
| 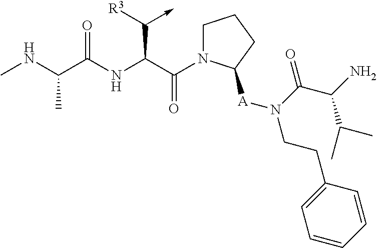 | 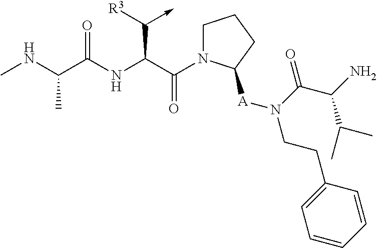 |
| A = CH₂; R³ = H or Me | A = CH₂; R³ = H or Me |
| 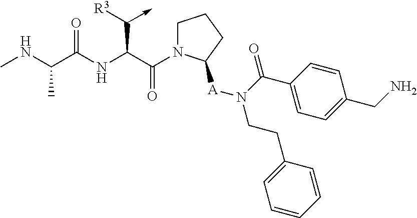 | 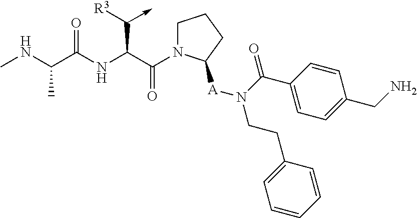 |
| A = CH₂; R³ = H or Me | A = CH₂; R³ = H or Me |
| 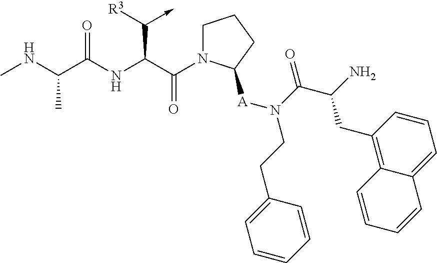 | 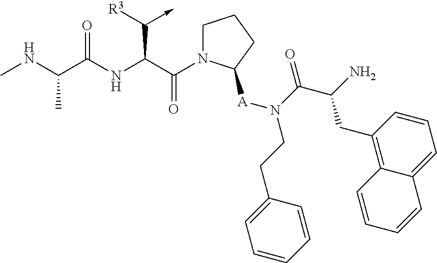 |
| A = CH₂; R³ = H or Me | A = CH₂; R³ = H or Me |
| 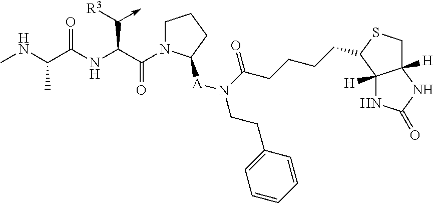 | 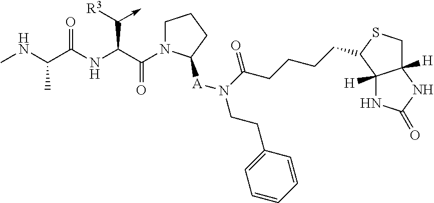 |
| A = CH₂; R³ = H or Me | A = CH₂; R³ = H or Me |
| 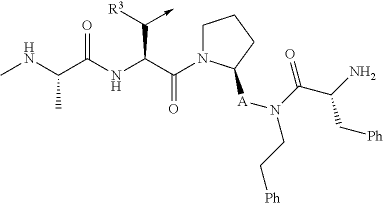 | 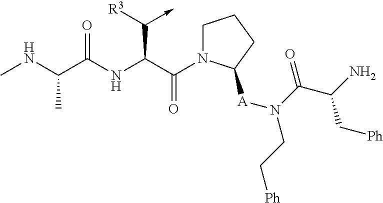 |
| A = CH₂; R³ = H or Me | A = CH₂; R³ = H or Me |
| 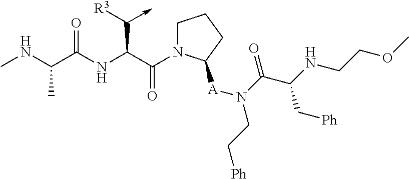 | 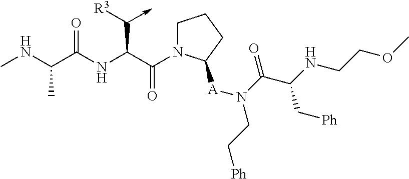 |
| A = CH₂; R³ = H or Me | A = CH₂; R³ = H or Me |
| 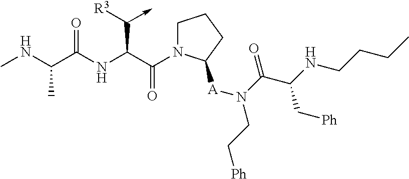 | 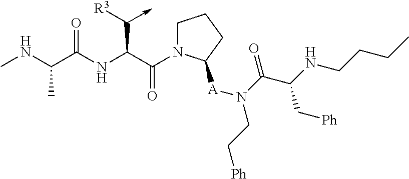 |
| A = CH₂; R³ = H or Me | A = CH₂; R³ = H or Me |
| 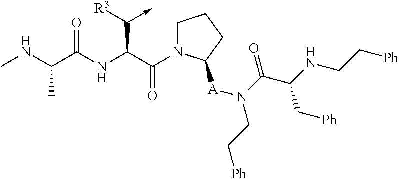 | 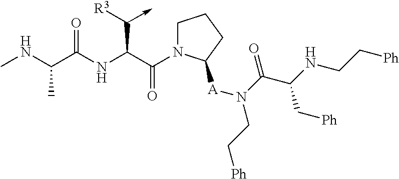 |
| A = CH₂; R³ = H or Me | A = CH₂; R³ = H or Me |
| 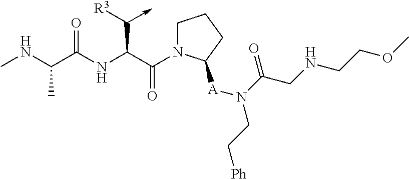 | 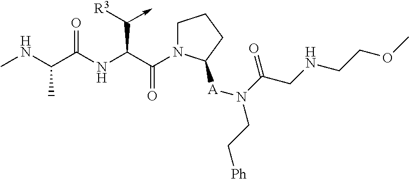 |
| A = CH₂; R³ = H or Me | A = CH₂; R³ = H or Me |
| 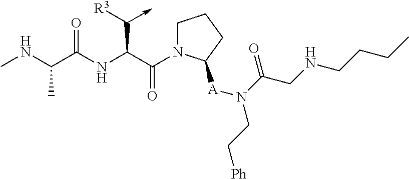 | 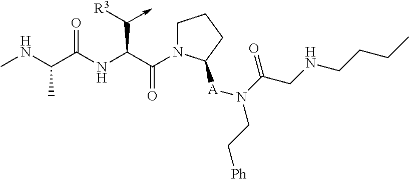 |
| A = CH₂; R³ = H or Me | A = CH₂; R³ = H or Me |
| 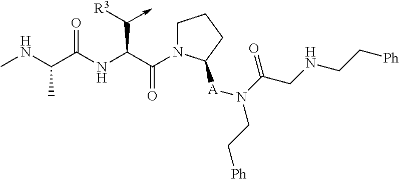 | 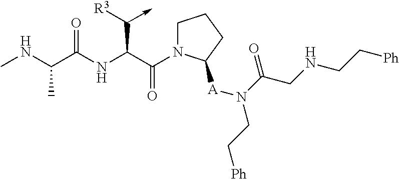 |
| A = CH₂; R³ = H or Me | A = CH₂; R³ = H or Me |
| 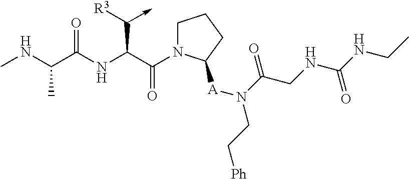 | 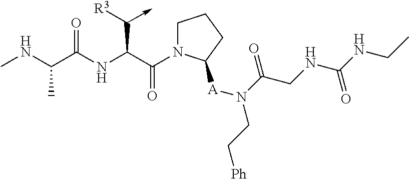 |
| A = CH₂; R³ = H or Me | A = CH₂; R³ = H or Me |
| 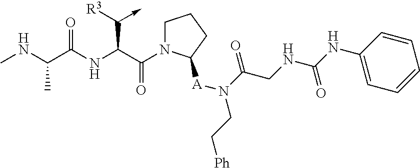 | 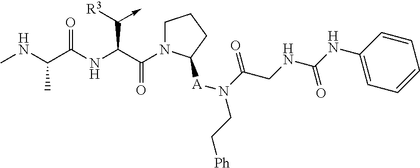 |
| A = CH₂; R³ = H or Me | A = CH₂; R³ = H or Me |
| 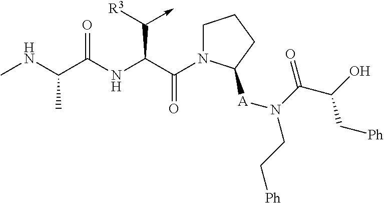 | 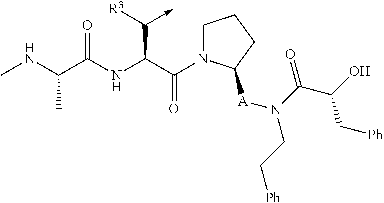 |
| A = CH₂; R³ = H or Me | A = CH₂; R³ = H or Me |
| 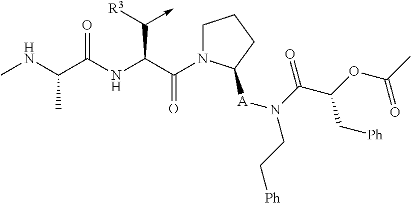 | 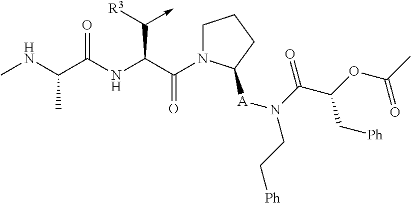 |
| A = CH₂; R³ = H or Me | A = CH₂; R³ = H or Me |
| 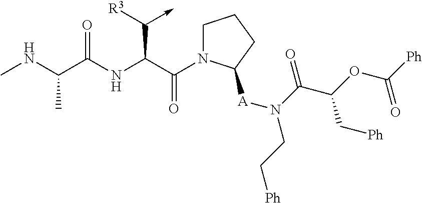 | 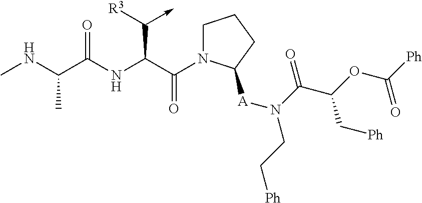 |
| A = CH₂; R³ = H or Me | A = CH₂; R³ = H or Me |
| 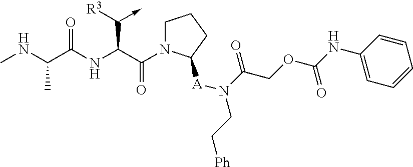 | 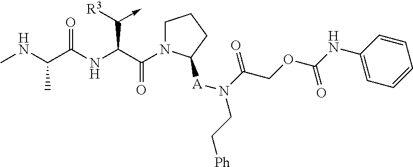 |
| A = CH₂; R³ = H or Me | A = CH₂; R³ = H or Me |
| 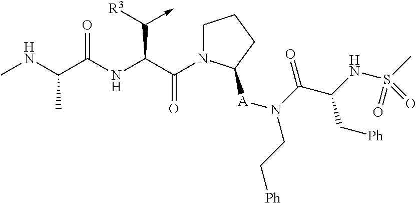 | 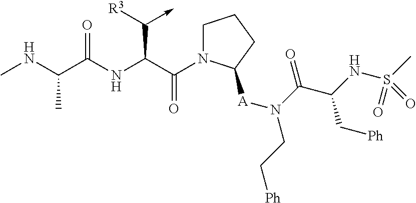 |
| A = CH₂; R³ = H or Me | A = CH₂; R³ = H or Me |
| 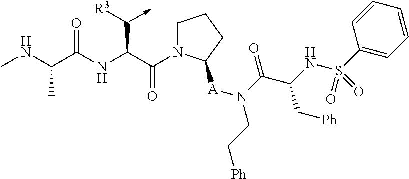 | 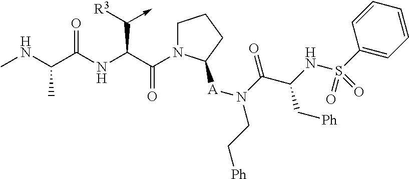 |
| A = CH₂; R³ = H or Me | A = CH₂; R³ = H or Me |
| 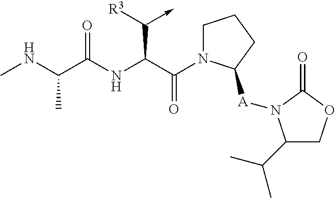 | 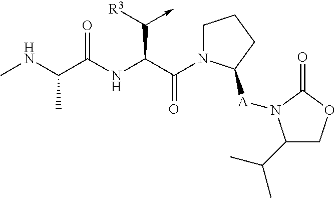 |
| A = CH₂; R³ = H or Me | A = CH₂; R³ = H or Me |
| 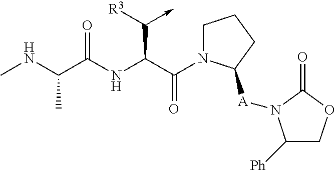 | 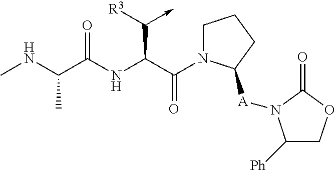 |
| A = CH₂; R³ = H or Me | A = CH₂; R³ = H or Me |
| 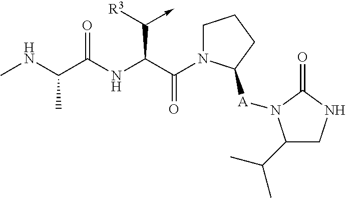 | 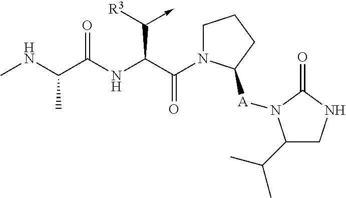 |
| A = CH₂; R³ = H or Me | A = CH₂; R³ = H or Me |
| 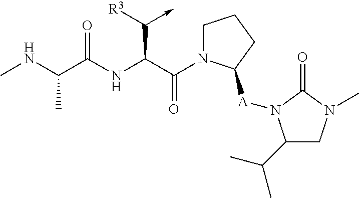 | 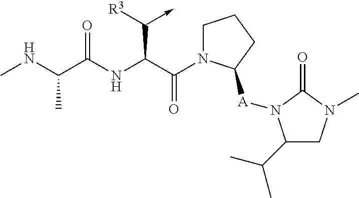 |
| A = CH₂; R³ = H or Me | A = CH₂; R³ = H or Me |
| 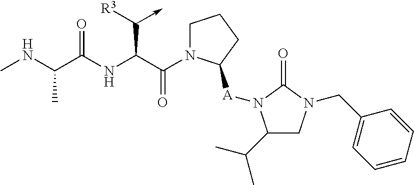 | 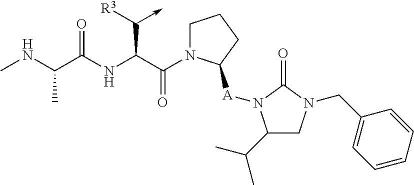 |
| A = CH₂; R³ = H or Me | A = CH₂; R³ = H or Me |
| 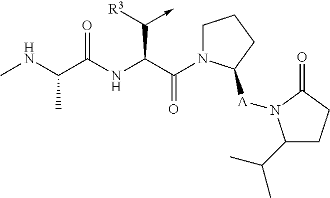 | 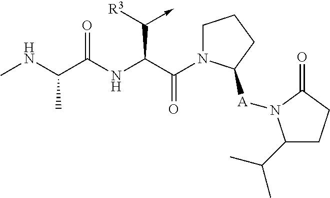 |
| A = CH₂; R³ = H or Me | A = CH₂; R³ = H or Me |
| 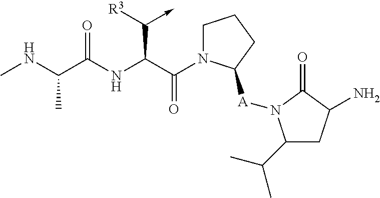 | 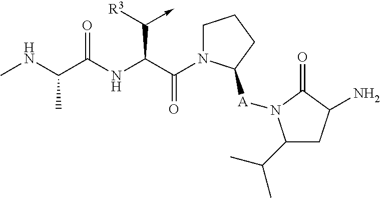 |
| A = CH₂; R³ = H or Me | A = CH₂; R³ = H or Me |

TABLE 3

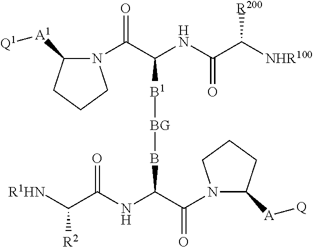

wherein R¹, R¹⁰⁰, R², R²⁰⁰, B, B¹, BG, A, A¹ are as defined herein;
Q and Q¹ are independently defined as NR⁴R⁵, wherein R⁵ is defined as
herein and R⁴ is chosen from the following:
R⁴

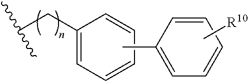

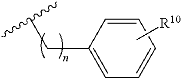

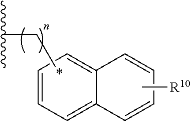

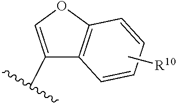

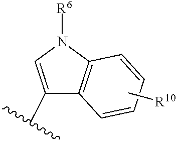

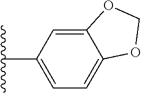

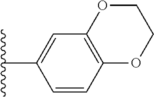

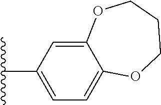

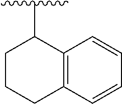

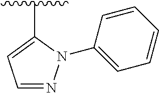

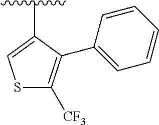

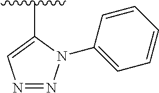

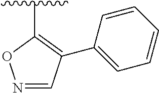

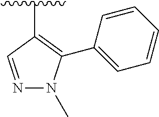

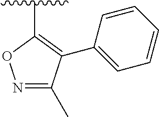

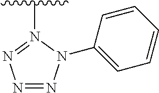

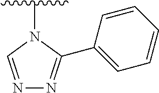

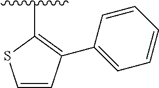

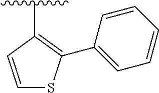

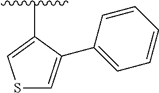

TABLE 4

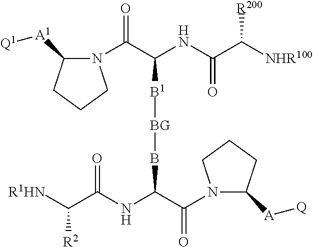

wherein R¹, R¹⁰⁰, R², R²⁰⁰, B, B¹, BG, A, A¹ are as defined herein;
Q and Q¹ are independently defined as OR¹¹, and OR¹¹
chosen from the following:
R¹¹

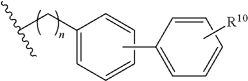

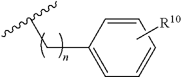

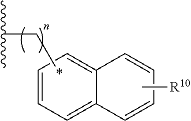

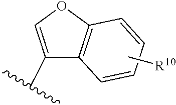

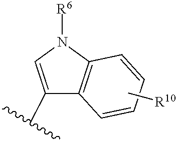

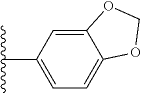

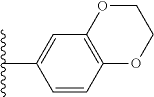

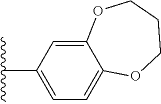

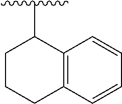

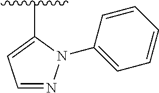

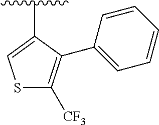

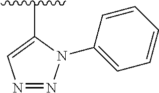

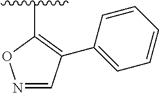

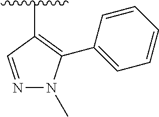

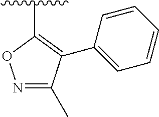

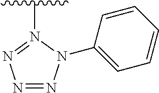

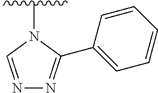

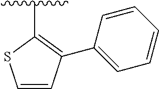

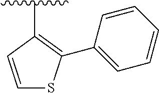

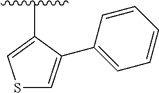

TABLE 5

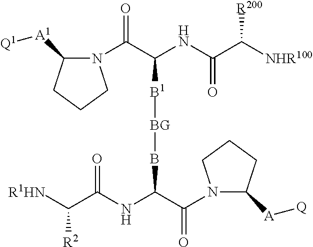

wherein R¹, R¹⁰⁰, R², R²⁰⁰, B, B¹, BG, A, A¹ are as defined herein;
Q and Q¹ are independently defined as S(O)ₘR¹¹, and R¹¹
is chosen from the following:
R¹¹

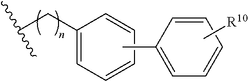

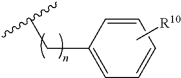

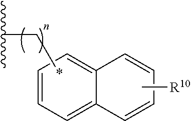

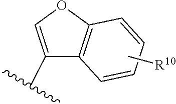

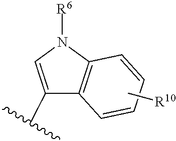

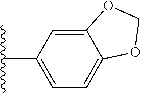

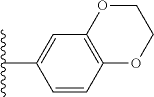

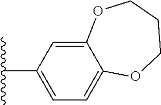

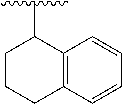

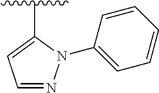

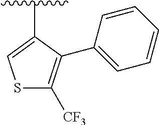

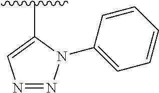

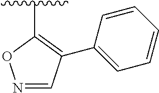

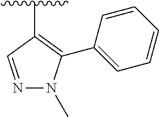

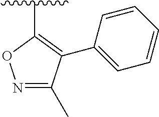

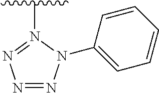

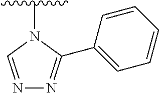

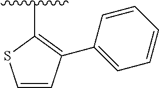

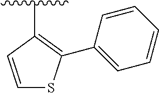

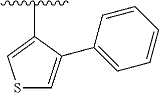

### Molecular Constructs for Expression

GST-XIAP BIR3RING: XIAP coding sequence amino acids 246-497 cloned into PGEX2T1 via BamH1 and AVA I. The plasmid was transformed into E. coli DH5α for use in protein expression and purification.

GST-HIAP2 (cIAP-1) BIR 3: HIAP2 coding sequence from amino acids 251-363 cloned into PGex4T3 via BamH1 and XhoI. The plasmid was transformed into E. coli DH5α for use in protein expression and purification.

GST-HIAP1 (cIAP-2) BIR 3: HIAP1 coding sequence from amino acids 236-349, cloned into PGex4T3 via BamH1 and XhoI. The plasmid was transformed into E. coli DH5α for use in protein expression and purification.

GST-linker BIR 2 BIR3Ring: XIAP coding sequence from amino acids 93-497 cloned into PGex4T1 via BamH1 and XhoI. Amino acids 93-497 were amplified from full length XIAP in pGex4t3, using the primers: TTAATAGGATCCATCAACGGCTTTTATC and GCTGCATGTGTGTCAGAGG, using standard PCR conditions. The PCR fragment was TA cloned into pCR-2.1 (invitrogen). Linker BIR 2 BIR 3Ring was subcloned into pGex4T1 by BamHI/XhoI digestion. The plasmid was transformed into E. coli DH5α for use in protein expression and purification.

Full-length human XIAP, AEG plasmid number 23. XIAP coding sequence amino acids 1-497 cloned into GST fusion vector, PGEX4T1 via BamH1 and Xho I restriction sites. A gift from Bob Korneluk and Peter Liston. The plasmid was transformed into E. coli DH5α for use in protein purification.

GST-XIAP linker BIR 2: XIAP linker BIR 2 coding sequence from amino acids 93-497 cloned into pGex4T3 via BamHI and XhoI. The plasmid was transformed into E. coli DH5α for use in protein expression and purification.

### Synthesis of Fluorescent Probe for FP Assay

A fluorescent peptide probe, Fmoc-Ala-Val-Pro-Phe-Tyr(t-Bu)-Leu-Pro-Gly(t-Bu)-Gly-OH was prepared using standard Fmoc chemistry on 2-chlorotrityl chloride resin (Int. J. Pept. Prot. Res. 38:555-561, 1991). Cleavage from the resin was performed using 20% acetic acid in dichloromehane (DCM), which left the side chain still blocked. The C-terminal protected carboxylic acid was coupled to 4′-(aminomethyl)fluorescein (Molecular Probes, A-1351; Eugene, Oreg.) using excess diisopropylcarbodiimide (DIC) in dimethylformamide (DMF) at room temperature and was purified by silica gel chromatography (10% methanol in DCM). The N-terminal Fmoc protecting group was removed using piperidine (20%) in DMF, and purified by silica gel chromatography (20% methanol in DCM, 0.5% HOAc). Finally, the t-butyl side chain protective groups were removed using 95% trifluoroacetic acid containing 2.5% water and 2.5% triisopropyl silane. The peptide obtained displayed a single peak by HPLC (>95% pure).

### Expression and Purification of Recombinant Proteins

### A. Recombinant Proteins Expression

Glutathione S-transferase (GST) tagged proteins were expressed in Escherichia coli strains DH5-alpha. For expression full length XIAP, individual or combinations of XIAP-BIR domains, cIAP-1, cIAP-2 and Livin transformed bacteria were cultured overnight at 37° C. in Luria Broth (LB) medium supplemented with 50 ug/ml of ampicillin. The overnight culture was then diluted 25 fold into fresh LB ampicillin supplemented media and bacteria were grown up to A₆₀₀=0.6 then induced with 1 mM isopropyl-D-1-thiogalactopyranoside for 3 hours. Upon induction, cells were centrifuged at 5000 RPM for 10 minutes and the media was removed. Each pellet obtained from a 1 liter culture received 10 ml of lysis buffer (50 mM Tris-HCl, 200 mM NaCl, 1 mM DTT, 1 mM PMSF, 2 mg/ml of lysosyme, 100 □g/ml)), was incubated at 4° C. with gentle shaking. After 20 minutes of incubation, the cell suspension was placed at −80° C. overnight or until needed.

### B. Purification of Recombinant Proteins

For purification of recombinant proteins, the IPTG-induced cell lysate was thawed vortexed and then disrupted by flash freezing in liquid nitrogen two times with vortexing after each thaw. The cells were disrupted further by passing the extract four times through a Bio-Neb Cell disruptor device (Glas-col) set at 100 psi with Nitrogen gas. The extract was clarified by centrifugation at 4 C at 15000 RPM in a SS-34 Beckman rotor for 30 minutes. The resulting supernatant was then mixed with 2 ml of glutathione-Sepharose beads (Pharmacia) per 500 ml cell culture (per 1000 ml culture for full length XIAP) for 1 hour at 4 C. Afterwards, the beads were washed 3 times with 1× Tris-Buffered Saline (TBS) to remove unbound proteins. The retained proteins were eluted with 2 washes of 2 ml of 50 mM TRIS pH 8.0 containing 10 mM reduced glutathione. The eluted proteins were pooled and precipitated with 604 g/liter of ammonium sulfate and the resulting pellet re-suspended into an appropriate buffer. As judged by SDS-PAGE the purified proteins were >90% pure. The protein concentration of purified proteins was determined from the Bradford method.

His-tag proteins were expressed in the E. Coli strain in E. coli AD494 cells using a pet28ACPP32 construct. The soluble protein fraction was prepared as described above. For protein purification, the supernatant was purified by affinity chromatography using chelating-Sepharose (Pharmacia) charged with NiSO₄ according to the manufacturer's instructions. Purity of the eluted protein was >90% pure as determined by SDS-PAGE. The protein concentration of purified proteins was determined from the Bradford assay.

### Binding Assay

### Fluorescence Polarization-Based Competition Assay

For all assays, the fluorescence and fluorescence-polarization was evaluated using a Tecan Polarion instrument with the excitation filter set at 485 nm and the emission filter set at 535 nm. For each assay, the concentration of the target protein was first establish by titration of the selected protein in order to produce a linear dose-response signal when incubated alone in the presence of the fluorescent probe. Upon establishing these conditions, the compounds potency (IC₅₀) and selectivity, was assessed in the presence of a fix defined-amount of target protein and fluorescent probe and a 10 point serial dilution of the selected compounds. For each IC₅₀ curve, the assays were run as followed: 25 ul/well of diluted compound in 50 mM IVIES buffer pH 6.5 were added into a black 96 well plate then 25 ul/well of bovine serum albumin (BSA) at 0.5 mg/ml in 50 mM IVIES pH 6.5. Auto-fluorescence for each compound was first assessed by performing a reading of the compound/BSA solution alone. Then 25 ul of the fluorescein probe diluted into 50 mM IVIES containing 0.05 mg/ml BSA were added and a reading to detect quenching of fluorescein signal done. Finally 25 ul/well of the target or control protein (GST-BIRs) diluted at the appropriate concentration in 50 mM IVIES containing 0.05 mg/ml BSA were added and the fluorescence polarization evaluated.

### Determination of IC₅₀ and Inhibitory Constants

For each assay the relative polarization-fluorescence units were plotted against the final concentrations of compound and the IC₅₀ calculated using the Grad pad prism software and/or Cambridge soft. The kᵢ value were derived from the calculated IC₅₀ value as described above and according to the equation described in Nikolovska-Coleska, Z. (2004) Anal Biochem 332, 261-273.

### Caspase-3 Full Length XIAP, Linker BIR2 or Linker-BIR2-BIR3-RING Derepression Assay

In order to determine the relative activity of the selected compound against XIAP-Bir2, we setup an in vitro assay where caspase-3 was inhibited by GST fusion proteins of XIAP linker-bir2, XIAP Linker Bir2-Bir3-RING or full-length XIAP. Caspase 3 (0.125 ul) and 12.25-34.25 nM (final concentration) of GST-XIAP fusion protein (GST-Bir2, GST-Bir2Bir3RING or full-length XIAP) were co-incubated with serial dilutions of compound (200 uM-5 pM). Caspase 3 activity was measured by overlaying 25 ul of a 0.4 mM DEVD-AMC solution. Final reaction volume was 100 ul. All dilutions were performed in caspase buffer (50 mM Hepes pH 7.4, 100 mM NaCl, 10% sucrose, 1 mM EDTA, 10 mM DTT, 0.1% CHAPS (Stennicke, H. R., and Salvesen, G. S. (1997). Biochemical characteristics of caspase-3, -6, -7, and -8. J. Biol. Chem. 272, 25719-25723)

The fluorescent AMC released from the caspase-3 hydrolysis of the substrate was measured in a TECAN spectrophotometer at 360 nm excitation and 444 nm emission, after 15 minutes of incubation at room temperature. IC50 values were calculated on a one or two-site competition model using GraphPad v4.0, using the fluorescence values after 15 minutes of incubation plotted against the log 10 concentration of compound.

### Cell-Free Assay

### Caspase De-Repression Assay Using Cellular Extracts (Apoptosome)

100 ug of 293 cell S100 extract and 0.25 uM-2 uM of GST-XIAP fusion protein (XIAP-Bir3RING, XIAP-Bir2Bir3RING, or full-length XIAP) were co-incubated with serial dilutions of compound (40 uM-5 pM). Caspases present in the extracts were activated by adding 1 mM dATP, 0.1 mM ALLN, 133 ug Cytochrome C (final concentrations), and incubating at 37° C. for 25 minutes. All reactions and dilutions used S100 buffer (50 mM Pipes pH 7.0, 50 mM KCl, 0.5 mM EGTA pH 8.0, 2 mM MgCl2 supplemented with 1/1000 dilutions of 2 mg/ml Cytochalisin B, 2 mg/ml Chymotstatin, Leupeptin, Pepstatin, Antipain, 0.1M PMSF, 1M DTT). Final reaction volume was 30 ul. Caspase-3 activity was measured by overlaying 30 ul of a 0.4 mM DEVD-AMC solution. released AMC cleavage was measured in a TECAN spectrophotometer at 360 nm excitation and 444 nm emission, on a kinetic cycle of 1 hour with readings taken every 5 minutes. Caspase activity was calculated as Vₒ of AMC fluorescence/sec. Caspase de-repression by our compounds was compared to fully activated extract and activated extract repressed by the presence of XIAP fusion protein.

### Cell Culture and Cell Death Assays

### A. Cell Culture

MDA-MD-231 (breast) and SKOV-3 cancer cells were cultured in RPMI1640 media supplemented with 10% FBS and 100 units/mL of Penicillin and Streptomycin.

### B. Assays

Viability assays were done on a number of cells including MDA-MB-231, SKOV-3, H460, PC3, HCT-116, and SW480 cells. Cells were seeded in 96 well plates at a respective density of 5000 and 2000 cells per well and incubated at 37° C. in presence of 5% CO₂ for 24 hours. Selected compounds were diluted into the media at various concentration ranging from 0.01 uM up to 100 uM. Diluted compounds were added onto the MDA-MB-231 cells. For the MDA-MB-231 SKOV3, H460, PC3, HCT-116, and SW480 cells, the compounds were added either alone or in presence of 1-3 ng/ml of TRAIL. After 72 hours cellular viability was evaluated by MTT based assays. ED₅₀s of select compounds against MDA and SKOV3 cell lines are presented in the Table below:

| | SKOV-3 | MDA-MB-231 |
|---|---|---|
| Compound | ED₅₀ (nM) | ED₅₀ (nM) |
| 1 | 131 | 17 |
| 2 | | 150 |
| 3 | 67 | |
| 4 | 37 | 159 |
| 5 | 57 | |
| 6 | 557 | |
| 7 | 136 | |
| 8 | 1917 | |

Apoptosis Assay: Measurement of Caspase-3 Activity from Cultured Cells.

One day, prior to the treatment, 10 000 cells per well were plated in a white tissue culture treated 96 well plate with 100 ul of media. On the day of compound treatment, compounds were diluted with cell culture media to a working stock concentration of 2× and 100 ul of diluted compound were added to each well and the plate was incubated for 5 h at 37° C. in presence of 5% CO₂. Upon incubation, the plate was washed twice with 200 ul of cold TRIS Buffered Saline (TBS) buffer. Cells were lysed with 50 ul of Caspase assay buffer (20 mM Tris-HCl pH 7.4, 0.1% NP-40, 0.1% Chaps, 1 mM DTT, 0.1 mM EDTA, 0.1 mM PMSF, 2 mg/ml Chymotstatin, Leupeptin, Pepstatin, Antipain) then incubated at 4° C. with shaking for 30 minutes. 45 ul of Caspase assay buffer and 5 ul of Ac-DEVD-AMC at 1 mg/ml were added to each well, the plate shaken and incubated for 16 h at 37° C. The amount of release AMC was measured in a TECAN spectrophotometer at with the excitation and emission filter set at 360 nm and 444 nm. The percentage of Caspase-3 activity was expressed in comparison of the signal obtained with the non-treated cells.

### Cellular Biochemistry:

### A. Detection of XIAP and PARP/Caspase-3/Caspase-9

Detection of cell expressed XIAP and PARP were done by western blotting. Cells were plated at 300 000 cells/well in a 60 mm wells (6 wells plate dish). The next day the cells were treated with selected compound at the indicated concentration. 24 hours later cells the trypsinized cells, pelleted by centrifugation at 1800 rpm at 4° C. The resulting pellet was rinsed twice with cold TBS. The final washed pellet of cells was the lysed with 250 ul Lysis buffer (NP-40, glycerol, 1% of a protease inhibitor cocktail (Sigma)), placed at 4° C. for 25 min with gentle shaking. The cells extract was centrifuged at 4° C. for 10 min at 10 000 rpm. Both the supernatant and the pellet were kept for western blotting analysis as described below. From the supernatant, the protein content was evaluated and about 50 ug of protein was fractionated onto a 10% SDS-PAGE. Pellets were washed with the lysis buffer and re-suspend into 50 ul of Lamelli buffer 1×, boiled and fractionated on SDS-PAGE. Upon electrophoresis each gel was electro-transferred onto a nitrocellulose membrane at 0.6 A for 2 hours. Membrane non-specific sites were blocked for 1 hours with 5% Skim milk in TBST (TBS containing 0.1% (v/v) Tween-20) at RT. For protein immuno-detection, membranes were incubated overnight with primary antibodies raised against XIAP clone 48 obtained from Becton-Dickison) or PARP: obtained from Cell signal or caspase-3 or caspase-9 primary antibodies were incubated at 4° C. with shaking at dilutions as follows:
XIAP clone 80 (Becton-Dickinson) . . . 1/2500
PARP (Cell Signal) . . . 1/2500
Caspase 3 (Sigma) . . . 1/1500
Caspase 9 (Upstate) . . . 1/1000

Upon overnight incubation, the membranes received three washes of 15 min in TBST then were incubated for 1 hour at room temperature in the presence of a secondary antibody coupled with HRP-enzyme (Chemicon) and diluted at 1/5 000. Upon incubation each membrane were washed three times with TBST and the immunoreactive bands were detected by addition of a luminescent substrate (ECL kit Amersham) and capture of signal on a X-RAY film for various time of exposure. Active compounds were shown to induce the cleavage of PARP and XIAP as well as to translocate XIAP into an insoluble compartment.

### Hollow Fiber Model

Hollow fiber in vivo model were used to demonstrate in vivo efficacy of selected compounds against selected cell lines as single agent therapy or in combination with selected cytotoxic agents. At day 1, selected cell lines were cultured and the fiber filled at a cell density of about 40,000 cells/fiber. At the day of operation (day 4), three fibers are implanted sub-cutaneous into 28-35 Nu/Nu CD-1 male mice. On day 5, mice start to receive daily injection via sub-cutaneous route of control vehicle or vehicle containing the selected compound at the appropriate concentration and/or injection of cytotoxic agent via intra-peritoneal route. Upon 7 days of non-consecutive treatments, the animals are sacrificed, each fiber is removed and the metabolic viability of the remaining cells determined by MTT assay. Efficacy of the compound is define as the difference between the vehicle-treated animal and the animal treated with the compound alone or the compound given in combination of the cytotoxic agent

### Combination Anti-Cancer Therapy In Vivo

Female nude mice received 2×10 HCT-116 subdermally on the right flank. On day 26, when tumors were ˜90 mm, animals were assigned to groups using a balanced design based on tumor size. At that time mitomycin-C and Compound 23 treatment was initiated. Mitomycin-C was administered ip at 1 mg/kg, Monday through Friday for two weeks. Compound 23 was given iv at 1 or 5 mg/kg five times per week for the duration of the experiment. Tumor measurements were taken twice weekly. As illustrated in FIG. 1, compound 23 showed an increasing anti-tumor effect in combination with mitomycin-C with increasing dose, with 5 mg/kg showing superior anti-tumor effects compared to the 1 mg/kg dose.

### Pharmacokinetic Studies

Selected compounds were dissolved into normal saline or appropriate vehicle and given at various doses using different route of administration, including intravenous bolus, intravenous infusion, oral and subcutaneous injection.

All literature, patents, published patent applications cited herein are hereby incorporated by reference.

### Other Embodiments

While specific embodiments have been described, those skilled in the art will recognize many alterations that could be made within the spirit of the disclosure, which is defined solely according to the following claims.

What is claimed is:

1. A compound represented by Formula I: 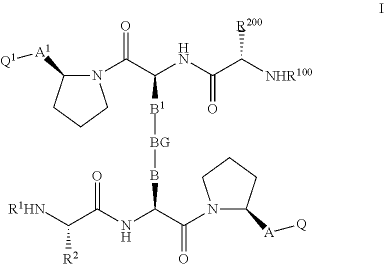
wherein
n is 0 or 1;
m is 0, 1 or 2;
p is 1 or 2;
Y is NH, O or S;
B and B¹ are independently C₁-C₆ alkyl;
BG is
1) -aryl-C₂-C₄ alkynyl-(X)ₙ—,
2) -aryl-(C₂-C₄ alkynyl)-aryl-,
3) biphenyl ether, or
4) biphenyl;
X is aryl or C₁-C₆ alkyl-O—;
R¹, R¹⁰⁰, R² and R²⁰⁰ are independently selected from:
1) H, or
2) C₁-C₆ alkyl optionally substituted with one or more R⁶ substituents;
Q and Q¹ are each independently
1) NR⁴R⁵,
2) OR¹¹, or
3) S(O)ₘR¹¹; or
Q and Q¹ are each independently 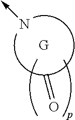
wherein G is a 5, 6 or 7 membered ring which optionally incorporates one or more heteroatoms chosen from S, N or O, the ring being optionally substituted with one or more R¹² substituents;
R⁴ and R⁵ are each independently
H,
haloalkyl,
←C₁-C₆ alkyl,
←C₂-C₆ alkenyl,
←C₂-C₄ alkynyl,
←C₃-C₇ cycloalkyl,
←C₃-C₇ cycloalkenyl,
←aryl,
←heteroaryl,
←heterocyclyl,
←heterobicyclyl,
←C(O)—R¹¹,
←C(O)O—R¹¹,
←C(═Y)NR⁸R⁹, or
←S(O)₂—R¹¹,
wherein the alkyl, alkenyl, alkynyl, cycloalkyl, cycloalkenyl is optionally substituted with one or more R⁶ substituents; and wherein the aryl, heteroaryl, heterocyclyl, and heterobicyclyl is optionally substituted with one or more R¹⁰ substituents;
R⁶ is
halogen,
NO₂,
CN,
haloalkyl,
C₁-C₆ alkyl,
C₂-C₆ alkenyl,
C₂-C₄ alkynyl,
C₃-C₇ cycloalkyl,
C₃-C₇ cycloalkenyl,
aryl,
heteroaryl,
heterocyclyl,
heterobicyclyl,
OR⁷,
S(O)ₘR⁷,
NR⁸R⁹,
NR⁸S(O)₂R¹¹,
COR⁷,
C(O)OR⁷,
CONR⁸R⁹,
S(O)₂NR⁸R⁹
OC(O)R⁷,
OC(O)Y—R¹¹,
SC(O)R⁷, or
NC(Y)NR⁸R⁹,
wherein the aryl, heteroaryl, heterocyclyl, and heterobicyclyl is optionally substituted with one or more R¹⁰ substituents;
R⁷ is
H,
haloalkyl,
C₁-C₆ alkyl,
C₂-C₆ alkenyl,
C₂-C₄ alkynyl,
C₃-C₇ cycloalkyl,
C₃-C₇ cycloalkenyl,
aryl,
heteroaryl,
heterocyclyl,
heterobicyclyl,
R⁸R⁹NC(═Y), or
C₁-C₆ alkyl-C₂-C₄ alkenyl, or
C₁-C₆ alkyl-C₂-C₄ alkynyl,
wherein the alkyl, alkenyl, alkynyl, cycloalkyl, cycloalkenyl is optionally substituted with one or more R⁶ substituents; and wherein the aryl, heteroaryl, heterocyclyl, and heterobicyclyl is optionally substituted with one or more R¹⁰ substituents;
R⁸ and R⁹ are each independently
H,
2) haloalkyl,
3) C₁-C₆ alkyl,
4) C₂-C₆ alkenyl,
5) C₂-C₄ alkynyl,
6) C₃-C₇ cycloalkyl,
7) C₃-C₇ cycloalkenyl,
8) aryl,
9) heteroaryl,
10) heterocyclyl,
11) heterobicyclyl,
12) C(O)R¹¹,
13) C(O)Y—R¹¹, or
14) S(O)₂—R¹¹,
wherein the alkyl, alkenyl, alkynyl, cycloalkyl, cycloalkenyl is optionally substituted with one or more R⁶ substituents; and wherein the aryl, heteroaryl, heterocyclyl, and heterobicyclyl is optionally substituted with one or more R¹⁰ substituents;
or R⁸ and R⁹ together with the nitrogen atom to which they are bonded form a five, six or seven membered heterocyclic ring optionally substituted with one or more R⁶ substituents;
R¹⁰ is
halogen,
NO₂,
CN,
B(OR¹³)(OR¹⁴),
C₁-C₆ alkyl,
C₂-C₆ alkenyl,
C₂-C₄ alkynyl,
C₃-C₇ cycloalkyl,
C₃-C₇ cycloalkenyl,
haloalkyl,
OR⁷,
NR⁸R⁹,
SR⁷,
COR⁷,
C(O)O R⁷,
S(O)ₘR⁷,
CONR⁸R⁹,
S(O)₂NR⁸R⁹,
aryl,
heteroaryl,
heterocyclyl, or
heterobicyclyl,
wherein the alkyl, alkenyl, alkynyl, cycloalkyl, and cycloalkenyl is optionally substituted with one or more R⁶ substituents;
R¹¹ is
haloalkyl,
C₁-C₆ alkyl,
C₂-C₆ alkenyl,
C₂-C₄ alkynyl,
C₃-C₇ cycloalkyl,
C₃-C₇ cycloalkenyl,
aryl,
heteroaryl,
heterocyclyl, or
heterobicyclyl,
wherein the alkyl, alkenyl, alkynyl, cycloalkyl, cycloalkenyl is optionally substituted with one or more R⁶ substituents; and wherein the aryl, heteroaryl, heterocyclyl, and heterobicyclyl is optionally substituted with one or more R¹⁰ substituents;
R¹² is
haloalkyl,
C₁-C₆ alkyl,
C₂-C₆ alkenyl,
C₂-C₄ alkynyl,
C₃-C₇ cycloalkyl,
C₃-C₇ cycloalkenyl,
aryl,
heteroaryl,
heterocyclyl,
heterobicyclyl,
C(O)—R¹¹,
C(O)O—R¹¹,
C(O)NR⁸R⁹,
S(O)ₘ—R¹¹, or
C(═Y)NR⁸R⁹,
wherein the alkyl, alkenyl, alkynyl, cycloalkyl, cycloalkenyl is optionally substituted with one or more R⁶ substituents; and wherein the aryl, heteroaryl, heterocyclyl, and heterobicyclyl is optionally substituted with one or more R¹⁰ substituents;
R¹³ and R¹⁴ are each independently
1) H, or
2) C₁-C₆ alkyl; or
R¹³ and R¹⁴ are combined to form a heterocyclic ring or a heterobicyclyl ring; and
A and A¹ are both CH₂ or C═O;
or a prodrug; or the compound of Formula I is labeled with a detectable label or an affinity tag.

2. The compound, according to claim 1, in which A and A¹ are both CH₂.

3. The compound, according to claim 1, in which A and A¹ are both C═O.

4. The compound, according to claim 1, in which A is CH₂ and A¹ is C═O.

5. The compound, according to claim 1, comprising compounds of Formula 1a through 1c: 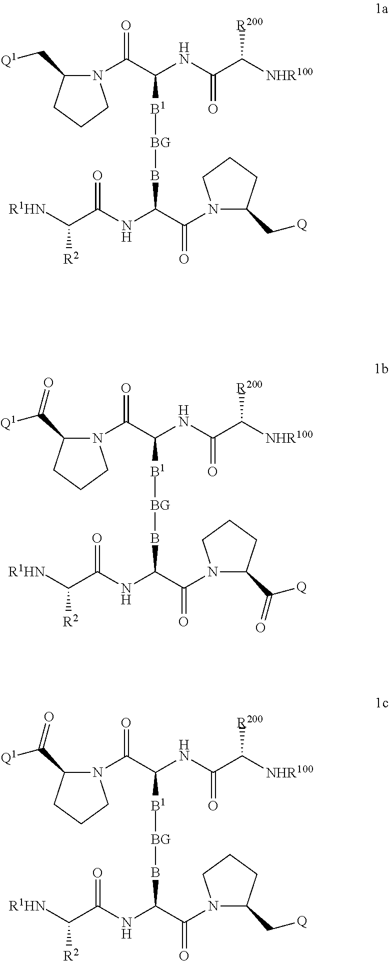
wherein BG, B, B¹, Q, Q¹, R¹, R¹⁰⁰, R² and R²⁰⁰ are as defined in claim 1.

6. The compound, according to claim 1, in which B and B¹ are both C₁-C₄ alkyl.

7. The compound, according to claim 1, in which BG is -aryl-C₂-C₄ alkynyl-(X)ₙ—.

8. The compound, according to claim 1, comprising compounds of Formula 1d and 1e: 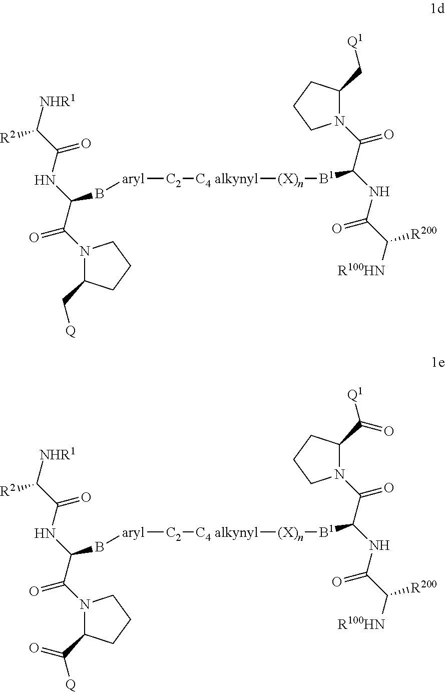
wherein X, n, B, B¹, Q, Q¹, R¹, R¹⁰⁰, R² and R²⁰⁰ are as defined in claim 1.

9. The compound, according to claim 1, in which BG is biphenyl ether, or biphenyl.

10. The compound, according to claim 1, comprising compounds of Formula 1f and 1 g: 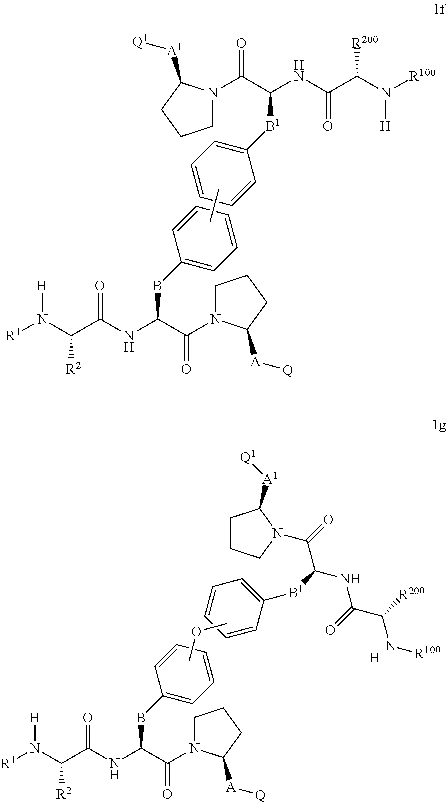
wherein A, A¹, B, B¹, Q, Q¹, R¹, R¹⁰⁰, R² and R²⁰⁰ are as defined in claim 1.

11. The compound, according to claim 1, in which BG is 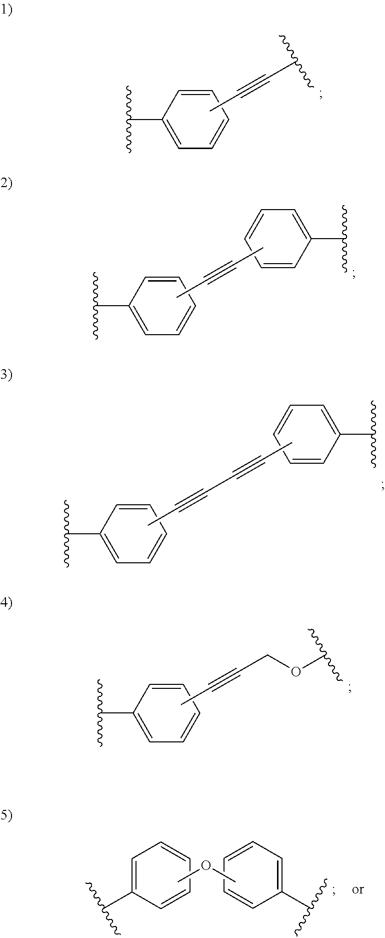
6) biphenyl.

12. The compound, according to claim 1, comprising compounds of Formula 1 h, 1i, 1j, 1k, 1l and 1m: 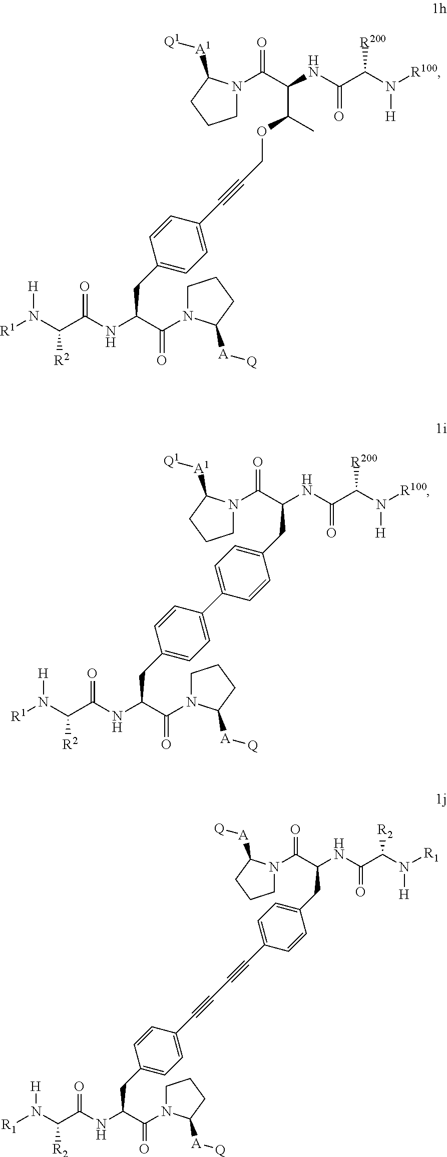 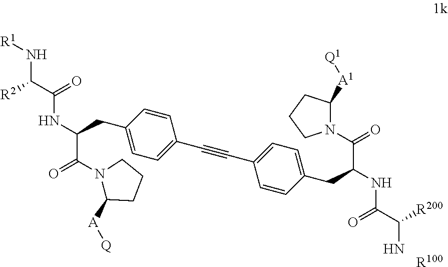
wherein A, A¹, Q, Q¹, R¹, R¹⁰⁰, R² and R²⁰⁰ are as defined in claim 1.

13. The compound, according to claim 1, in which R¹ and R¹⁰⁰ are both C₁-C₆ alkyl.

14. The compound, according to claim 13, in which R¹ and R¹⁰ are both CH₃.

15. The compound, according to claim 1, in which R² and R²⁰⁰ are both C₁-C₆ alkyl.

16. The compound, according to claim 15 in which R² and R²⁰⁰ are both CH₃.

17. The compound, according to claim 1, in which Q and Q¹ are both NR⁴R⁵.

18. The compound, according to claim 1, in which A and A¹ are both C═O, Q and Q¹ are both NR⁴R⁵, R⁴ is H and
R⁵ is selected from
1) haloalkyl,
2) ←C₁-C₆ alkyl,
3) ←C₂-C₆ alkenyl,
4) ←C₂-C₄ alkynyl,
5) ←C₃-C₇ cycloalkyl,
6) ←C₃-C₇ cycloalkenyl,
7) ←aryl,
8) ←heteroaryl,
9) ←heterocyclyl, or
10) ←heterobicyclyl,
wherein the alkyl, alkenyl, alkynyl, cycloalkyl, cycloalkenyl is optionally substituted with one or more R⁶ substituents; and wherein the aryl, heteroaryl, heterocyclyl, and heterobicyclyl is optionally substituted with one or more R¹⁰ substituents.

19. The compound, according to claim 1, in which A and A¹ are both CH₂, Q and Q¹ are both NR⁴R⁵, and R⁴ and R⁵ are each independently
1) H,
2) haloalkyl,
3) ←C₁-C₆ alkyl,
4) ←C₂-C₆ alkenyl,
5) ←C₂-C₄ alkynyl,
6) ←C₃-C₇ cycloalkyl,
7) ←C₃-C₇ cycloalkenyl,
8) ←aryl,
9) ←heteroaryl,
10) ←heterocyclyl,
11) ←heterobicyclyl,
12) ←C(O)—R¹¹,
13) ←C(O)O—R¹¹,
13) ←C(═Y)NR⁸R⁹, or
14) ←S(O)₂—R¹¹,
wherein the alkyl, alkenyl, alkynyl, cycloalkyl, cycloalkenyl is optionally substituted with one or more R⁶ substituents; and wherein the aryl, heteroaryl, heterocyclyl, and heterobicyclyl is optionally substituted with one or more R¹⁰ substituents.

20. The compound, according to claim 19, in which R¹¹ is
1) haloalkyl,
2) C₁-C₆ alkyl,
3) C₂-C₆ alkenyl,
4) C₂-C₄ alkynyl,
5) aryl,
6) heteroaryl,
7) heterocyclyl, or
8) heterobicyclyl,
wherein the alkyl, alkenyl, alkynyl is optionally substituted with one or more R⁶ substituents; and wherein the aryl, heteroaryl, heterocyclyl, and heterobicyclyl is optionally substituted with one or more R¹⁰ substituents.

21. The compound, according to claim 1, in which R⁶ is
1) halogen,
2) NO₂,
3) CN,
4) aryl,
5) heteroaryl,
6) heterocyclyl,
7) heterobicyclyl,
8) OR⁷,
9) SR⁷, or
10) NR⁸R⁹,
wherein the aryl, heteroaryl, heterocyclyl, and heterobicyclyl is optionally substituted with one or more R¹⁰ substituents.

22. The compound, according to claim 1, in which R⁸ and R⁹ are each independently
1) H,
2) haloalkyl,
3) C₁-C₆ alkyl,
4) C₂-C₆ alkenyl,
5) C₂-C₄ alkynyl,
6) C₃-C₇ cycloalkyl, or
7) C₃-C₇ cycloalkenyl,
wherein the alkyl, alkenyl, alkynyl, cycloalkyl, cycloalkenyl is optionally substituted with one or more R⁶ substituents.

23. The compound, according to claim 1, in which R¹⁰ is
1) halogen,
2) NO₂,
3) CN,
4) haloalkyl,
5) OR⁷,
6) NR⁸R⁹, or
7) SR⁷.

24. A compound, according to claim 1, selected from the group consisting of:

| Compound | Structure |
|---|---|
| 1 | 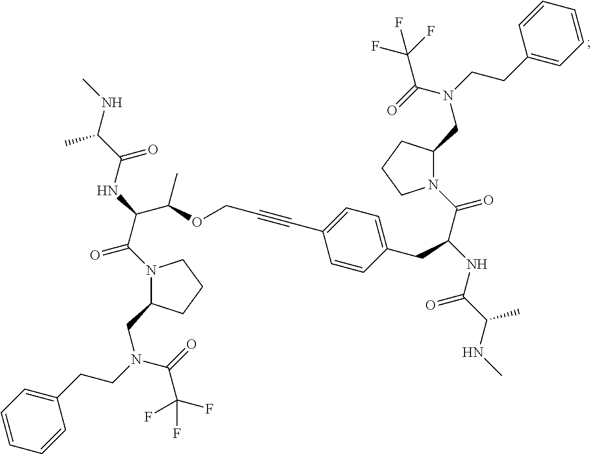 |
| 2 | 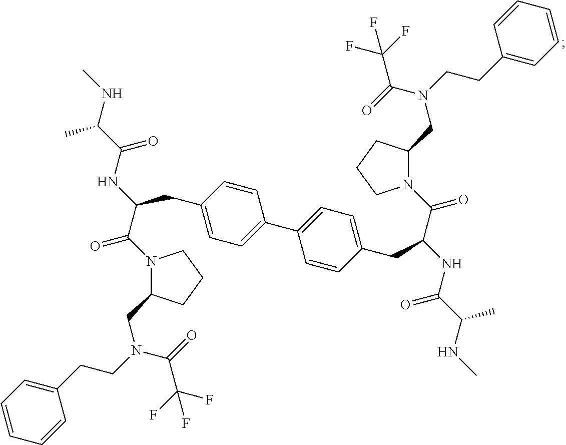 |
| 3 | 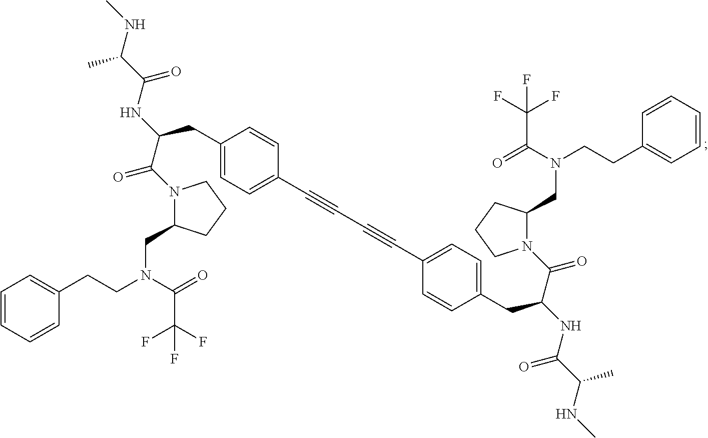 |
| 4 | 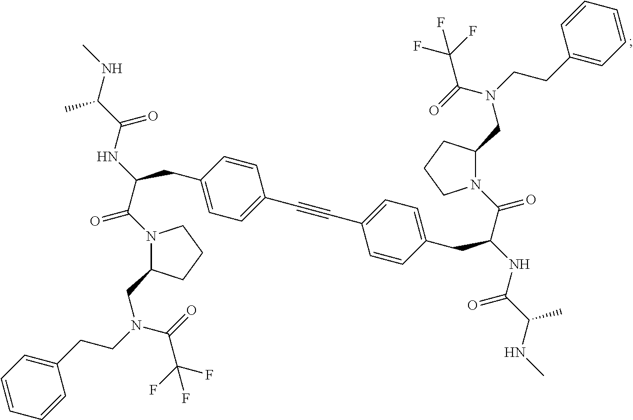 |
| 5 | 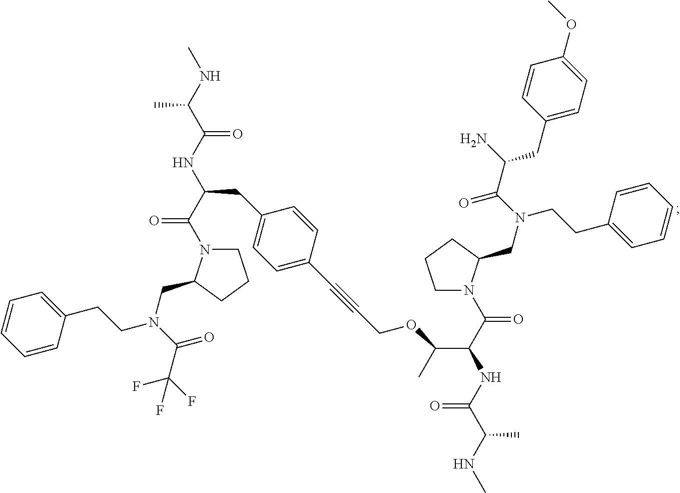 |
| 6 | 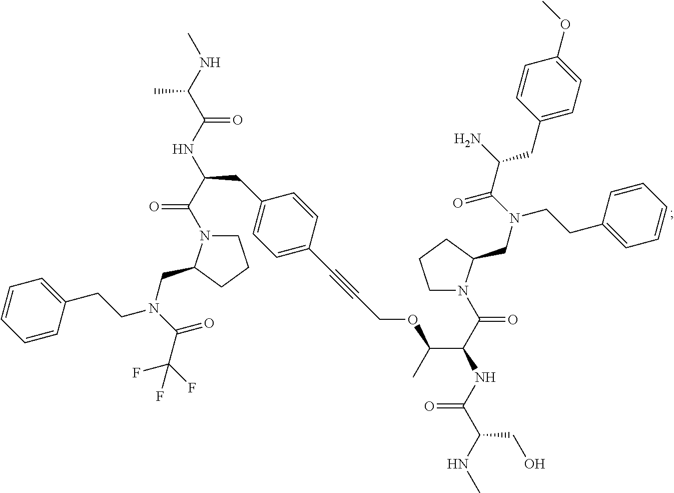 |
| 7 | 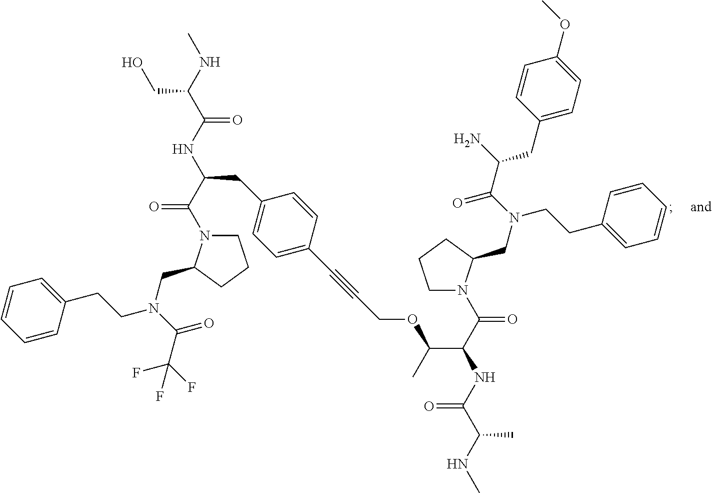 |
| 8 | 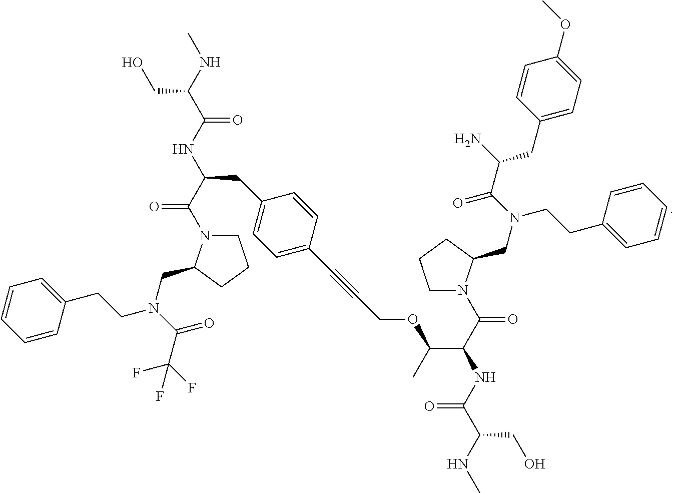 |

25. The compound, according to claim 1, is a salt.

26. The compound, according to claim 1, is a pharmaceutically acceptable salt.

27. An intermediate compound represented by Formula 2(iii): 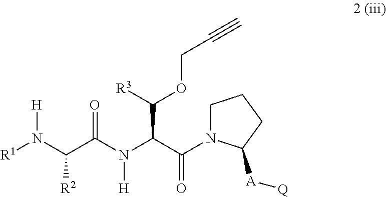
wherein:
R¹ and R² are independently selected from:
1) H, or
2) C₁-C₆ alkyl optionally substituted with one or more R⁶ substituents;
Q is
1) NR⁴R⁵,
2) OR¹¹, or
3) S(O)ₘR¹¹; or 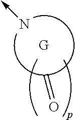
wherein G is a 5, 6 or 7 membered ring which optionally incorporates one or more heteroatoms chosen from S, N or O, the ring being optionally substituted with one or more R¹² substituents;
R⁴ and R⁵ are each independently
H,
haloalkyl,
←C₁-C₆ alkyl,
←C₂-C₆ alkenyl,
←C₂-C₄ alkynyl,
←C₃-C₇ cycloalkyl,
←C₃-C₇ cycloalkenyl,
←aryl,
←heteroaryl,
←heterocyclyl,
←heterobicyclyl,
←C(O)—R¹¹,
←C(O)O—R¹¹,
←C(═Y)NR⁸R⁹, or
←S(O)₂—R¹¹,
wherein the alkyl, alkenyl, alkynyl, cycloalkyl, cycloalkenyl is optionally substituted with one or more R⁶ substituents; and wherein the aryl, heteroaryl, heterocyclyl, and heterobicyclyl is optionally substituted with one or more R¹⁰ substituents;
R⁶ is
halogen,
NO₂,
CN,
haloalkyl,
C₁-C₆ alkyl,
C₂-C₆ alkenyl,
C₂-C₄ alkynyl,
C₃-C₇ cycloalkyl,
C₃-C₇ cycloalkenyl,
aryl,
heteroaryl,
heterocyclyl,
heterobicyclyl,
OR⁷,
S(O)ₘR⁷,
NR⁸R⁹,
NR⁸S(O)₂R¹¹,
COR⁷,
C(O)OR⁷,
CONR⁸R⁹,
S(O)₂NR⁸R⁹,
OC(O)R⁷,
OC(O)Y—R¹¹,
SC(O)R⁷, or
NC(Y)NR⁸R⁹,
wherein the aryl, heteroaryl, heterocyclyl, and heterobicyclyl is optionally substituted with one or more R¹⁰ substituents;
R⁷ is
H,
haloalkyl,
C₁-C₆ alkyl,
C₂-C₆ alkenyl,
C₂-C₄ alkynyl,
C₃-C₇ cycloalkyl,
C₃-C₇ cycloalkenyl,
aryl,
heteroaryl,
heterocyclyl,
heterobicyclyl,
R⁸R⁹NC(═Y), or
C₁-C₆ alkyl-C₂-C₄ alkenyl, or
C₁-C₆ alkyl-C₂-C₄ alkynyl,
wherein the alkyl, alkenyl, alkynyl, cycloalkyl, cycloalkenyl is optionally substituted with one or more R⁶ substituents; and wherein the aryl, heteroaryl, heterocyclyl, and heterobicyclyl is optionally substituted with one or more R¹⁰ substituents;
R⁸ and R⁹ are each independently
1) H,
2) haloalkyl,
3) C₁-C₆ alkyl,
4) C₂-C₆ alkenyl,
5) C₂-C₄ alkynyl,
6) C₃-C₇ cycloalkyl,
7) C₃-C₇ cycloalkenyl,
8) aryl,
9) heteroaryl,
10) heterocyclyl,
11) heterobicyclyl,
12) C(O)R¹¹,
13) C(O)Y—R¹¹, or
14) S(O)₂—R¹¹,
wherein the alkyl, alkenyl, alkynyl, cycloalkyl, cycloalkenyl is optionally substituted with one or more R⁶ substituents; and wherein the aryl, heteroaryl, heterocyclyl, and heterobicyclyl is optionally substituted with one or more R¹⁰ substituents;
or R⁸ and R⁹ together with the nitrogen atom to which they are bonded form a five, six or seven membered heterocyclic ring optionally substituted with one or more R⁶ substituents;
R¹⁰ is
halogen,
NO₂,
CN,
B(OR¹³)(OR¹⁴),
C₁-C₆ alkyl,
C₂-C₆ alkenyl,
C₂-C₄ alkynyl,
C₃-C₇ cycloalkyl,
C₃-C₇ cycloalkenyl,
haloalkyl,
OR⁷,
NR⁸R⁹,
SR⁷,
COR⁷,
C(O)OR⁷,
S(O)ₘR⁷,
CONR⁸R⁹,
S(O)₂NR⁸R⁹,
aryl,
heteroaryl,
heterocyclyl, or
heterobicyclyl,
wherein the alkyl, alkenyl, alkynyl, cycloalkyl, and cycloalkenyl is optionally substituted with one or more R⁶ substituents;
R¹¹ is
haloalkyl,
C₁-C₆ alkyl,
C₂-C₆ alkenyl,
C₂-C₄ alkynyl,
C₃-C₇ cycloalkyl,
C₃-C₇ cycloalkenyl,
aryl,
heteroaryl,
heterocyclyl, or
heterobicyclyl,
wherein the alkyl, alkenyl, alkynyl, cycloalkyl, cycloalkenyl is optionally substituted with one or more R⁶ substituents; and wherein the aryl, heteroaryl, heterocyclyl, and heterobicyclyl is optionally substituted with one or more R¹⁰ substituents;
R¹² is
haloalkyl,
C₁-C₆ alkyl,
C₂-C₆ alkenyl,
C₂-C₄ alkynyl,
C₃-C₇ cycloalkyl,
C₃-C₇ cycloalkenyl,
aryl,
heteroaryl,
heterocyclyl,
heterobicyclyl,
C(O)—R¹¹,
C(O)O—R¹¹,
C(O)NR⁸R⁹,
S(O)ₘ—R¹¹, or
C(═Y)NR⁸R⁹,
wherein the alkyl, alkenyl, alkynyl, cycloalkyl, cycloalkenyl is optionally substituted with one or more R⁶ substituents; and wherein the aryl, heteroaryl, heterocyclyl, and heterobicyclyl is optionally substituted with one or more R¹⁰ substituents;
R¹³ and R¹⁴ are each independently
1) H, or
2) C₁-C₆ alkyl; or
R¹³ and R¹⁴ are combined to form a heterocyclic ring or a heterobicyclyl ring;
R³ is H or methyl; and
A is CH₂ or C═O.

28. An intermediate compound represented by Formula 6(i): 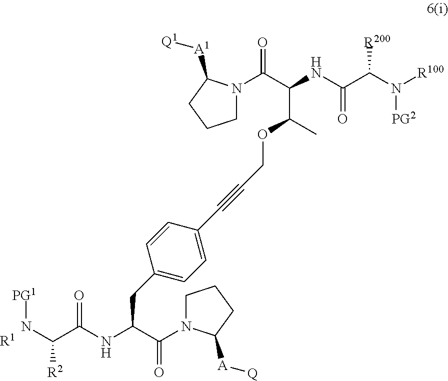
wherein PG¹ and PG² are protecting groups, and wherein
R¹, R¹⁰⁰, R² and R²⁰⁰ are independently selected from:
1) H, or
2) C₁-C₆ alkyl optionally substituted with one or more R⁶ substituents;
Q and Q¹ are each independently
1) NR⁴R⁵,
2) OR¹¹, or
3) S(O)ₘR¹¹; or
Q and Q¹ are each independently 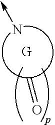
wherein G is a 5, 6 or 7 membered ring which optionally incorporates one or more heteroatoms chosen from S, N or O, the ring being optionally substituted with one or more R¹² substituents;
R⁴ and R⁵ are each independently
H,
haloalkyl,
←C₁-C₆ alkyl,
←C₂-C₆ alkenyl,
←C₂-C₄ alkynyl,
←C₃-C₇ cycloalkyl,
←C₃-C₇ cycloalkenyl,
←aryl,
←heteroaryl,
←heterocyclyl,
←heterobicyclyl,
←C(O)—R¹¹,
←C(O)O—R¹¹,
←C(═Y)NR⁸R⁹, or
←S(O)₂—R¹¹,
wherein the alkyl, alkenyl, alkynyl, cycloalkyl, cycloalkenyl is optionally substituted with one or more R⁶ substituents; and wherein the aryl, heteroaryl, heterocyclyl, and heterobicyclyl is optionally substituted with one or more R¹⁰ substituents;
R⁶ is
halogen,
NO₂,
CN,
haloalkyl,
C₁-C₆ alkyl,
C₂-C₆ alkenyl,
C₂-C₄ alkynyl,
C₃-C₇ cycloalkyl,
C₃-C₇ cycloalkenyl,
aryl,
heteroaryl,
heterocyclyl,
heterobicyclyl,
OR⁷,
S(O)ₘR⁷,
NR⁸R⁹,
NR⁸S(O)₂R¹¹,
COR⁷,
C(O)OR⁷,
CONR⁸R⁹,
S(O)₂NR⁸R⁹
OC(O)R⁷,
OC(O)Y—R¹¹,
SC(O)R⁷, or
NC(Y)NR⁸R⁹,
wherein the aryl, heteroaryl, heterocyclyl, and heterobicyclyl is optionally substituted with one or more R¹⁰ substituents;
R⁷ is
H,
haloalkyl,
C₁-C₆ alkyl,
C₂-C₆ alkenyl,
C₂-C₄ alkynyl,
C₃-C₇ cycloalkyl,
C₃-C₇ cycloalkenyl,
aryl,
heteroaryl,
heterocyclyl,
heterobicyclyl,
R⁸R⁹NC(═Y), or
C₁-C₆ alkyl-C₂-C₄ alkenyl, or
C₁-C₆ alkyl-C₂-C₄ alkynyl,
wherein the alkyl, alkenyl, alkynyl, cycloalkyl, cycloalkenyl is optionally substituted with one or more R⁶ substituents; and wherein the aryl, heteroaryl, heterocyclyl, and heterobicyclyl is optionally substituted with one or more R¹⁰ substituents;
R⁸ and R⁹ are each independently
H,
2) haloalkyl,
3) C₁-C₆ alkyl,
4) C₂-C₆ alkenyl,
5) C₂-C₄ alkynyl,
6) C₃-C₇ cycloalkyl,
7) C₃-C₇ cycloalkenyl,
8) aryl,
9) heteroaryl,
10) heterocyclyl,
11) heterobicyclyl,
12) C(O)R¹¹,
13) C(O)Y—R¹¹, or
14) S(O)₂—R¹¹,
wherein the alkyl, alkenyl, alkynyl, cycloalkyl, cycloalkenyl is optionally substituted with one or more R⁶ substituents; and wherein the aryl, heteroaryl, heterocyclyl, and heterobicyclyl is optionally substituted with one or more R¹⁰ substituents;
or R⁸ and R⁹ together with the nitrogen atom to which they are bonded form a five, six or seven membered heterocyclic ring optionally substituted with one or more R⁶ substituents;
R¹⁰ is
halogen,
NO₂,
CN,
B(OR¹³)(OR¹⁴),
C₁-C₆ alkyl,
C₂-C₆ alkenyl,
C₂-C₄ alkynyl,
C₃-C₇ cycloalkyl,
C₃-C₇ cycloalkenyl,
haloalkyl,
OR⁷,
NR⁸R⁹,
SR⁷,
COR⁷,
C(O)OR⁷,
S(O)ₘR⁷,
CONR⁸R⁹,
S(O)₂NR⁸R⁹,
aryl,
heteroaryl,
heterocyclyl, or
heterobicyclyl,
wherein the alkyl, alkenyl, alkynyl, cycloalkyl, and cycloalkenyl is optionally substituted with one or more R⁶ substituents;
R¹¹ is
haloalkyl,
C₁-C₆ alkyl,
C₂-C₆ alkenyl,
C₂-C₄ alkynyl,
C₃-C₇ cycloalkyl,
C₃-C₇ cycloalkenyl,
aryl,
heteroaryl,
heterocyclyl, or
heterobicyclyl,
wherein the alkyl, alkenyl, alkynyl, cycloalkyl, cycloalkenyl is optionally substituted with one or more R⁶ substituents; and wherein the aryl, heteroaryl, heterocyclyl, and heterobicyclyl is optionally substituted with one or more R¹⁰ substituents;
R¹² is
haloalkyl,
C₁-C₆ alkyl,
C₂-C₆ alkenyl,
C₂-C₄ alkynyl,
C₃-C₇ cycloalkyl,
C₃-C₇ cycloalkenyl,
aryl,
heteroaryl,
heterocyclyl,
heterobicyclyl,
C(O)—R¹¹,
C(O)O—R¹¹,
C(O)NR⁸R⁹,
S(O)ₘ—R¹¹, or
C(═Y)NR⁸R⁹,
wherein the alkyl, alkenyl, alkynyl, cycloalkyl, cycloalkenyl is optionally substituted with one or more R⁶ substituents; and wherein the aryl, heteroaryl, heterocyclyl, and heterobicyclyl is optionally substituted with one or more R¹⁰ substituents;
R¹³ and R¹⁴ are each independently
1) H, or
2) C₁-C₆ alkyl; or
R¹³ and R¹⁴ are combined to form a heterocyclic ring or a heterobicyclyl ring;
R³ is H or methyl; and
A and A¹ are both CH₂ or C═O.

29. An intermediate compound represented by Formula 7(i): 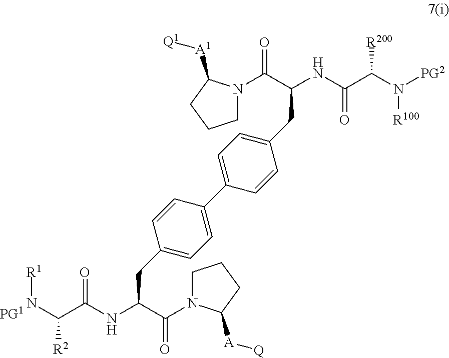
wherein PG¹ and PG² are protecting groups, and wherein
R¹, R¹⁰⁰, R² and R²⁰⁰ are independently selected from:
1) H, or
2) C₁-C₆ alkyl optionally substituted with one or more R⁶ substituents;
Q and Q¹ are each independently
1) NR⁴R⁵,
2) OR¹¹, or
3) S(O)ₘR¹¹; or
Q and Q¹ are each independently 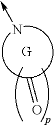
wherein G is a 5, 6 or 7 membered ring which optionally incorporates one or more heteroatoms chosen from S, N or O, the ring being optionally substituted with one or more R¹² substituents;
R⁴ and R⁵ are each independently
H,
haloalkyl,
←C₁-C₆ alkyl,
←C₂-C₆ alkenyl,
←C₂-C₄ alkynyl,
←C₃-C₇ cycloalkyl,
←C₃-C₇ cycloalkenyl,
←aryl,
←heteroaryl,
←heterocyclyl,
←heterobicyclyl,
←C(O)—R¹¹,
←C(O)O—R¹¹,
←C(═Y)NR⁸R⁹, or
←S(O)₂—R¹¹,
wherein the alkyl, alkenyl, alkynyl, cycloalkyl, cycloalkenyl is optionally substituted with one or more R⁶ substituents; and wherein the aryl, heteroaryl, heterocyclyl, and heterobicyclyl is optionally substituted with one or more R¹⁰ substituents;
R⁶ is
halogen,
NO₂,
CN,
haloalkyl,
C₁-C₆ alkyl,
C₂-C₆ alkenyl,
C₂-C₄ alkynyl,
C₃-C₇ cycloalkyl,
C₃-C₇ cycloalkenyl,
aryl,
heteroaryl,
heterocyclyl,
heterobicyclyl,
OR⁷,
S(O)ₘR⁷,
NR⁸R⁹,
NR⁸S(O)₂R¹¹,
COR⁷,
C(O)OR⁷,
CONR⁸R⁹,
S(O)₂NR⁸R⁹
OC(O)R⁷,
OC(O)Y—R¹¹,
SC(O)R⁷, or
NC(Y)NR⁸R⁹,
wherein the aryl, heteroaryl, heterocyclyl, and heterobicyclyl is optionally substituted with one or more R¹⁰ substituents;
R⁷ is
H,
haloalkyl,
C₁-C₆ alkyl,
C₂-C₆ alkenyl,
C₂-C₄ alkynyl,
C₃-C₇ cycloalkyl,
C₃-C₇ cycloalkenyl,
aryl,
heteroaryl,
heterocyclyl,
heterobicyclyl,
R⁸R⁹NC(═Y), or
C₁-C₆ alkyl-C₂-C₄ alkenyl, or
C₁-C₆ alkyl-C₂-C₄ alkynyl,
wherein the alkyl, alkenyl, alkynyl, cycloalkyl, cycloalkenyl is optionally substituted with one or more R⁶ substituents; and wherein the aryl, heteroaryl, heterocyclyl, and heterobicyclyl is optionally substituted with one or more R¹⁰ substituents;
R⁸ and R⁹ are each independently
H,
2) haloalkyl,
3) C₁-C₆ alkyl,
4) C₂-C₆ alkenyl,
5) C₂-C₄ alkynyl,
6) C₃-C₇ cycloalkyl,
7) C₃-C₇ cycloalkenyl,
8) aryl,
9) heteroaryl,
10) heterocyclyl,
11) heterobicyclyl,
12) C(O)R¹¹,
13) C(O)Y—R¹¹, or
14) S(O)₂—R¹¹,
wherein the alkyl, alkenyl, alkynyl, cycloalkyl, cycloalkenyl is optionally substituted with one or more R⁶ substituents; and wherein the aryl, heteroaryl, heterocyclyl, and heterobicyclyl is optionally substituted with one or more R¹⁰ substituents;
or R⁸ and R⁹ together with the nitrogen atom to which they are bonded form a five, six or seven membered heterocyclic ring optionally substituted with one or more R⁶ substituents;
R^{10 is}
halogen,
NO₂,
CN,
B(OR¹³)(OR¹⁴),
C₁-C₆ alkyl,
C₂-C₆ alkenyl,
C₂-C₄ alkynyl,
C₃-C₇ cycloalkyl,
C₃-C₇ cycloalkenyl,
haloalkyl,
OR⁷,
NR⁸R⁹,
SR⁷,
COR⁷,
C(O)OR⁷,
S(O)ₘR⁷,
CONR⁸R⁹,
S(O)₂NR⁸R⁹,
aryl,
heteroaryl,
heterocyclyl, or
heterobicyclyl,
wherein the alkyl, alkenyl, alkynyl, cycloalkyl, and cycloalkenyl is optionally substituted with one or more R⁶ substituents;
R¹¹ is
haloalkyl,
C₁-C₆ alkyl,
C₂-C₆ alkenyl,
C₂-C₄ alkynyl,
C₃-C₇ cycloalkyl,
C₃-C₇ cycloalkenyl,
aryl,
heteroaryl,
heterocyclyl, or
heterobicyclyl,
wherein the alkyl, alkenyl, alkynyl, cycloalkyl, cycloalkenyl is optionally substituted with one or more R⁶ substituents; and wherein the aryl, heteroaryl, heterocyclyl, and heterobicyclyl is optionally substituted with one or more R¹⁰ substituents;
R¹² is
haloalkyl,
C₁-C₆ alkyl,
C₂-C₆ alkenyl,
C₂-C₄ alkynyl,
C₃-C₇ cycloalkyl,
C₃-C₇ cycloalkenyl,
aryl,
heteroaryl,
heterocyclyl,
heterobicyclyl,
C(O)—R¹¹,
C(O)O—R¹¹,
C(O)NR⁸R⁹,
S(O)ₘ—R¹¹, or
C(═Y)NR⁸R⁹,
wherein the alkyl, alkenyl, alkynyl, cycloalkyl, cycloalkenyl is optionally substituted with one or more R⁶ substituents; and wherein the aryl, heteroaryl, heterocyclyl, and heterobicyclyl is optionally substituted with one or more R¹⁰ substituents;
R¹³ and R¹⁴ are each independently
1) H, or
2) C₁-C₆ alkyl; or
R¹³ and R¹⁴ are combined to form a heterocyclic ring or a heterobicyclyl ring;
R³ is H or methyl; and
A and A¹ are both CH₂ or C═O.

30. An intermediate compound represented by Formula 8(i): 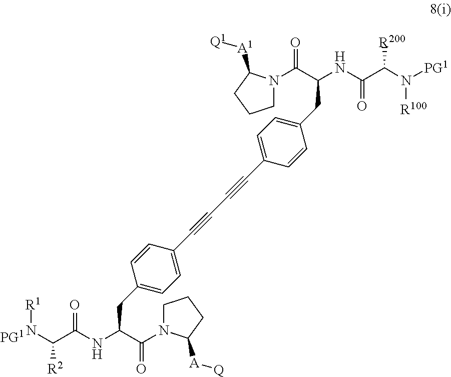
wherein PG¹ is a protecting group, and wherein
R¹, R¹⁰⁰, R² and R²⁰⁰ are independently selected from:
1) H, or
2) C₁-C₆ alkyl optionally substituted with one or more R⁶ substituents;
Q and Q¹ are each independently
1) NR⁴R⁵,
2) OR¹¹, or
3) S(O)ₘR¹¹; or
Q and Q¹ are each independently 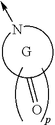
wherein G is a 5, 6 or 7 membered ring which optionally incorporates one or more heteroatoms chosen from S, N or O, the ring being optionally substituted with one or more R¹² substituents;
R⁴ and R⁵ are each independently
H,
haloalkyl,
←C₁-C₆ alkyl,
←C₂-C₆ alkenyl,
←C₂-C₄ alkynyl,
←C₃-C₇ cycloalkyl,
←C₃-C₇ cycloalkenyl,
←aryl,
←heteroaryl,
←heterocyclyl,
←heterobicyclyl,
←C(O)—R¹¹,
←C(O)O—R¹¹,
←C(═Y)NR⁸R⁹, or
←S(O)₂—R¹¹,
wherein the alkyl, alkenyl, alkynyl, cycloalkyl, cycloalkenyl is optionally substituted with one or more R⁶ substituents; and wherein the aryl, heteroaryl, heterocyclyl, and heterobicyclyl is optionally substituted with one or more R¹⁰ substituents;
R⁶ is
halogen,
NO₂,
CN,
haloalkyl,
C₁-C₆ alkyl,
C₂-C₆ alkenyl,
C₂-C₄ alkynyl,
C₃-C₇ cycloalkyl,
C₃-C₇ cycloalkenyl,
aryl,
heteroaryl,
heterocyclyl,
heterobicyclyl,
OR⁷,
S(O)ₘR⁷,
NR⁸R⁹,
NR⁸S(O)₂R¹¹,
COR⁷,
C(O)OR⁷,
CONR⁸R⁹,
S(O)₂NR⁸R⁹
OC(O)R⁷,
OC(O)Y—R¹¹,
SC(O)R⁷, or
NC(Y)NR⁸R⁹,
wherein the aryl, heteroaryl, heterocyclyl, and heterobicyclyl is optionally substituted with one or more R¹⁰ substituents;
R⁷ is
H,
haloalkyl,
C₁-C₆ alkyl,
C₂-C₆ alkenyl,
C₂-C₄ alkynyl,
C₃-C₇ cycloalkyl,
C₃-C₇ cycloalkenyl,
aryl,
heteroaryl,
heterocyclyl,
heterobicyclyl,
R⁸R⁹NC(═Y), or
C₁-C₆ alkyl-C₂-C₄ alkenyl, or
C₁-C₆ alkyl-C₂-C₄ alkynyl,
wherein the alkyl, alkenyl, alkynyl, cycloalkyl, cycloalkenyl is optionally substituted with one or more R⁶ substituents; and wherein the aryl, heteroaryl, heterocyclyl, and heterobicyclyl is optionally substituted with one or more R¹⁰ substituents;
R⁸ and R⁹ are each independently
H,
2) haloalkyl,
3) C₁-C₆ alkyl,
4) C₂-C₆ alkenyl,
5) C₂-C₄ alkynyl,
6) C₃-C₇ cycloalkyl,
7) C₃-C₇ cycloalkenyl,
8) aryl,
9) heteroaryl,
10) heterocyclyl,
11) heterobicyclyl,
12) C(O)R¹¹,
13) C(O)Y—R¹¹, or
14) S(O)₂—R¹¹,
wherein the alkyl, alkenyl, alkynyl, cycloalkyl, cycloalkenyl is optionally substituted with one or more R⁶ substituents; and wherein the aryl, heteroaryl, heterocyclyl, and heterobicyclyl is optionally substituted with one or more R¹⁰ substituents;
or R⁸ and R⁹ together with the nitrogen atom to which they are bonded form a five, six or seven membered heterocyclic ring optionally substituted with one or more R⁶ substituents;
R¹⁰ is
halogen,
NO₂,
CN,
B(OR¹³)(OR¹⁴),
C₁-C₆ alkyl,
C₂-C₆ alkenyl,
C₂-C₄ alkynyl,
C₃-C₇ cycloalkyl,
C₃-C₇ cycloalkenyl,
haloalkyl,
OR⁷,
NR⁸R⁹,
SR⁷,
COR⁷,
C(O)OR⁷,
S(O)ₘR⁷,
CONR⁸R⁹,
S(O)₂NR⁸R⁹,
aryl,
heteroaryl,
heterocyclyl, or
heterobicyclyl,
wherein the alkyl, alkenyl, alkynyl, cycloalkyl, and cycloalkenyl is optionally substituted with one or more R⁶ substituents;
R¹¹ is
haloalkyl,
C₁-C₆ alkyl,
C₂-C₆ alkenyl,
C₂-C₄ alkynyl,
C₃-C₇ cycloalkyl,
C₃-C₇ cycloalkenyl,
aryl,
heteroaryl,
heterocyclyl, or
heterobicyclyl,
wherein the alkyl, alkenyl, alkynyl, cycloalkyl, cycloalkenyl is optionally substituted with one or more R⁶ substituents; and wherein the aryl, heteroaryl, heterocyclyl, and heterobicyclyl is optionally substituted with one or more R¹⁰ substituents;
R¹² is
haloalkyl,
C₁-C₆ alkyl,
C₂-C₆ alkenyl,
C₂-C₄ alkynyl,
C₃-C₇ cycloalkyl,
C₃-C₇ cycloalkenyl,
aryl,
heteroaryl,
heterocyclyl,
heterobicyclyl,
C(O)—R¹¹,
C(O)O—R¹¹,
C(O)NR⁸R⁹,
S(O)ₘ—R¹¹, or
C(═Y)NR⁸R⁹,
wherein the alkyl, alkenyl, alkynyl, cycloalkyl, cycloalkenyl is optionally substituted with one or more R⁶ substituents; and wherein the aryl, heteroaryl, heterocyclyl, and heterobicyclyl is optionally substituted with one or more R¹⁰ substituents;
R¹³ and R¹⁴ are each independently
1) H, or
2) C₁-C₆ alkyl; or
R¹³ and R¹⁴ are combined to form a heterocyclic ring or a heterobicyclyl ring;
R³ is H or methyl; and
A and A¹ are both CH₂ or C═O.

31. A process for producing compounds of Formula 6(ii) above, the process comprising:
a) deprotecting PG¹ and PG² from intermediate 6(i), according to claim 28: 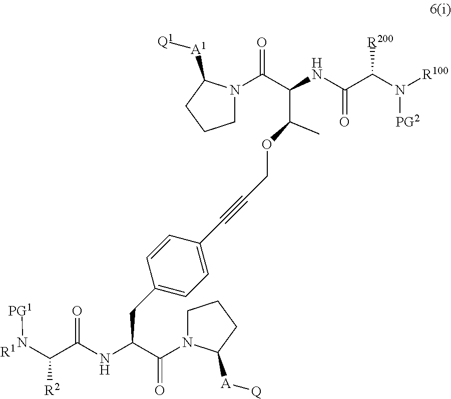
so as to produce compounds of Formula 6(ii): 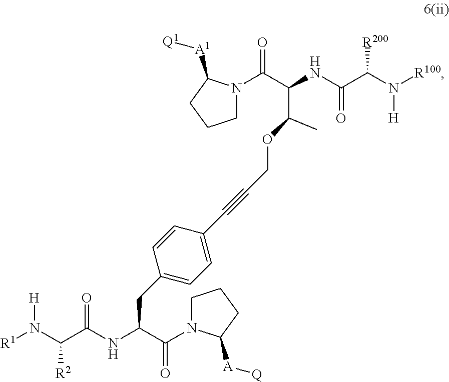
wherein PG¹ and PG² are protecting groups, and wherein
R¹, R¹⁰⁰, R² and R²⁰⁰ are independently selected from:
1) H, or
2) C₁-C₆ alkyl optionally substituted with one or more R⁶ substituents;
Q and Q¹ are each independently
1) NR⁴R⁵,
2) OR¹¹, or
3) S(O)ₘR¹¹; or
Q and Q¹ are each independently 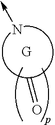
wherein G is a 5, 6 or 7 membered ring which optionally incorporates one or more heteroatoms chosen from S, N or O, the ring being optionally substituted with one or more R¹² substituents;
R⁴ and R⁵ are each independently
H,
haloalkyl,
←C₁-C₆ alkyl,
←C₂-C₆ alkenyl,
←C₂-C₄ alkynyl,
←C₃-C₇ cycloalkyl,
←C₃-C₇ cycloalkenyl,
←aryl,
←heteroaryl,
←heterocyclyl,
←heterobicyclyl,
←C(O)—R¹¹,
←C(O)O—R¹¹,
←C(═Y)NR⁸R⁹, or
←S(O)₂—R¹¹,
wherein the alkyl, alkenyl, alkynyl, cycloalkyl, cycloalkenyl is optionally substituted with one or more R⁶ substituents; and wherein the aryl, heteroaryl, heterocyclyl, and heterobicyclyl is optionally substituted with one or more R¹⁰ substituents;
R⁶ is
halogen,
NO₂,
CN,
haloalkyl,
C₁-C₆ alkyl,
C₂-C₆ alkenyl,
C₂-C₄ alkynyl,
C₃-C₇ cycloalkyl,
C₃-C₇ cycloalkenyl,
aryl,
heteroaryl,
heterocyclyl,
heterobicyclyl,
OR⁷,
S(O)ₘR⁷,
NR⁸R⁹,
NR⁸S(O)₂R¹¹,
COR⁷,
C(O)OR⁷,
CONR⁸R⁹,
S(O)₂NR⁸R⁹
OC(O)R⁷,
OC(O)Y—R¹¹,
SC(O)R⁷, or
NC(Y)NR⁸R⁹,
wherein the aryl, heteroaryl, heterocyclyl, and heterobicyclyl is optionally substituted with one or more R¹⁰ substituents;
R⁷ is
H,
haloalkyl,
C₁-C₆ alkyl,
C₂-C₆ alkenyl,
C₂-C₄ alkynyl,
C₃-C₇ cycloalkyl,
C₃-C₇ cycloalkenyl,
aryl,
heteroaryl,
heterocyclyl,
heterobicyclyl,
R⁸R⁹NC(═Y), or
C₁-C₆ alkyl-C₂-C₄ alkenyl, or
C₁-C₆ alkyl-C₂-C₄ alkynyl,
wherein the alkyl, alkenyl, alkynyl, cycloalkyl, cycloalkenyl is optionally substituted with one or more R⁶ substituents; and wherein the aryl, heteroaryl, heterocyclyl, and heterobicyclyl is optionally substituted with one or more R¹⁰ substituents;
R⁸ and R⁹ are each independently
H,
2) haloalkyl,
3) C₁-C₆ alkyl,
4) C₂-C₆ alkenyl,
5) C₂-C₄ alkynyl,
6) C₃-C₇ cycloalkyl,
7) C₃-C₇ cycloalkenyl,
8) aryl,
9) heteroaryl,
10) heterocyclyl,
11) heterobicyclyl,
12) C(O)R¹¹,
13) C(O)Y—R¹¹, or
14) S(O)₂—R¹¹,
wherein the alkyl, alkenyl, alkynyl, cycloalkyl, cycloalkenyl is optionally substituted with one or more R⁶ substituents; and wherein the aryl, heteroaryl, heterocyclyl, and heterobicyclyl is optionally substituted with one or more R¹⁰ substituents;
or R⁸ and R⁹ together with the nitrogen atom to which they are bonded form a five, six or seven membered heterocyclic ring optionally substituted with one or more R⁶ substituents;
R¹⁰ is
halogen,
NO₂,
CN,
B(OR¹³)(OR¹⁴),
C₁-C₆ alkyl,
C₂-C₆ alkenyl,
C₂-C₄ alkynyl,
C₃-C₇ cycloalkyl,
C₃-C₇ cycloalkenyl,
haloalkyl,
OR⁷,
NR⁸R⁹,
SR⁷,
COR⁷,
C(O)OR⁷,
S(O)ₘR⁷,
CONR⁸R⁹,
S(O)₂NR⁸R⁹,
aryl,
heteroaryl,
heterocyclyl, or
heterobicyclyl,
wherein the alkyl, alkenyl, alkynyl, cycloalkyl, and cycloalkenyl is optionally substituted with one or more R⁶ substituents;
R¹¹ is
haloalkyl,
C₁-C₆ alkyl,
C₂-C₆ alkenyl,
C₂-C₄ alkynyl,
C₃-C₇ cycloalkyl,
C₃-C₇ cycloalkenyl,
aryl,
heteroaryl,
heterocyclyl, or
heterobicyclyl,
wherein the alkyl, alkenyl, alkynyl, cycloalkyl, cycloalkenyl is optionally substituted with one or more R⁶ substituents; and wherein the aryl, heteroaryl, heterocyclyl, and heterobicyclyl is optionally substituted with one or more R¹⁰ substituents;
R¹² is
haloalkyl,
C₁-C₆ alkyl,
C₂-C₆ alkenyl,
C₂-C₄ alkynyl,
C₃-C₇ cycloalkyl,
C₃-C₇ cycloalkenyl,
aryl,
heteroaryl,
heterocyclyl,
heterobicyclyl,
C(O)—R¹¹,
C(O)O—R¹¹,
C(O)NR⁸R⁹,
S(O)ₘ—R¹¹, or
C(═Y)NR⁸R⁹,
wherein the alkyl, alkenyl, alkynyl, cycloalkyl, cycloalkenyl is optionally substituted with one or more R⁶ substituents; and wherein the aryl, heteroaryl, heterocyclyl, and heterobicyclyl is optionally substituted with one or more R¹⁰ substituents;
R¹³ and R¹⁴ are each independently
1) H, or
2) C₁-C₆ alkyl; or
R¹³ and R¹⁴ are combined to form a heterocyclic ring or a heterobicyclyl ring;
R³ is H or methyl; and
A and A¹ are both CH₂ or C═O.

32. A process for producing compounds of Formula 7(ii) above, the process comprising:
a) deprotecting PG¹ and PG² from intermediate 7(i), according to claim 29: 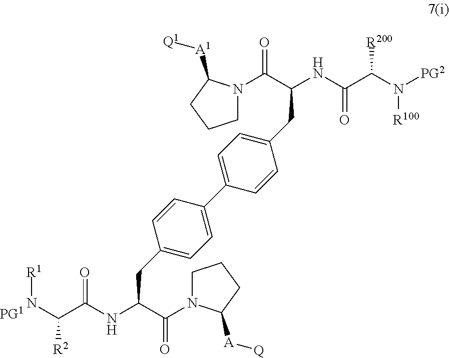
so as to produce compounds of Formula 7(ii): 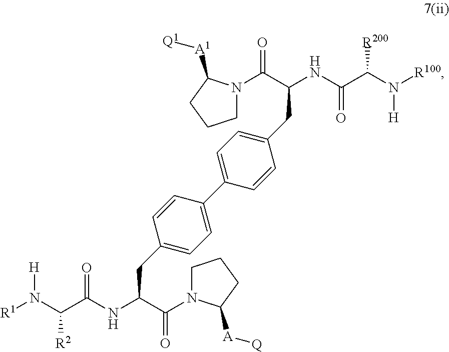
wherein PG¹ and PG² are protecting groups, and wherein
R¹, R¹⁰⁰, R² and R²⁰⁰ are independently selected from:
1) H, or
2) C₁-C₆ alkyl optionally substituted with one or more R⁶ substituents;
Q and Q¹ are each independently
1) NR⁴R⁵,
2) OR¹¹, or
3) S(O)ₘR¹¹; or
Q and Q¹ are each independently 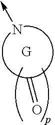
wherein G is a 5, 6 or 7 membered ring which optionally incorporates one or more heteroatoms chosen from S, N or O, the ring being optionally substituted with one or more R¹² substituents;
R⁴ and R⁵ are each independently
H,
haloalkyl,
←C₁-C₆ alkyl,
←C₂-C₆ alkenyl,
←C₂-C₄ alkynyl,
←C₃-C₇ cycloalkyl,
←C₃-C₇ cycloalkenyl,
←aryl,
←heteroaryl,
←heterocyclyl,
←heterobicyclyl,
←C(O)—R¹¹,
←C(O)O—R¹¹,
←C(═Y)NR⁸R⁹, or
←S(O)₂—R¹¹,
wherein the alkyl, alkenyl, alkynyl, cycloalkyl, cycloalkenyl is optionally substituted with one or more R⁶ substituents; and wherein the aryl, heteroaryl, heterocyclyl, and heterobicyclyl is optionally substituted with one or more R¹⁰ substituents;
R⁶ is
halogen,
NO₂,
CN,
haloalkyl,
C₁-C₆ alkyl,
C₂-C₆ alkenyl,
C₂-C₄ alkynyl,
C₃-C₇ cycloalkyl,
C₃-C₇ cycloalkenyl,
aryl,
heteroaryl,
heterocyclyl,
heterobicyclyl,
OR⁷,
S(O)ₘR⁷,
NR⁸R⁹,
NR⁸S(O)₂R¹¹,
COR⁷,
C(O)OR⁷,
CONR⁸R⁹,
S(O)₂NR⁸R⁹
OC(O)R⁷,
OC(O)Y—R¹¹,
SC(O)R⁷, or
NC(Y)NR⁸R⁹,
wherein the aryl, heteroaryl, heterocyclyl, and heterobicyclyl is optionally substituted with one or more R¹⁰ substituents;
R⁷ is
H,
haloalkyl,
C₁-C₆ alkyl,
C₂-C₆ alkenyl,
C₂-C₄ alkynyl,
C₃-C₇ cycloalkyl,
C₃-C₇ cycloalkenyl,
aryl,
heteroaryl,
heterocyclyl,
heterobicyclyl,
R⁸R⁹NC(═Y), or
C₁-C₆ alkyl-C₂-C₄ alkenyl, or
C₁-C₆ alkyl-C₂-C₄ alkynyl, wherein the alkyl, alkenyl, alkynyl, cycloalkyl, cycloalkenyl is optionally substituted with one or more R⁶ substituents; and wherein the aryl, heteroaryl, heterocyclyl, and heterobicyclyl is optionally substituted with one or more R¹⁰ substituents;
R⁸ and R⁹ are each independently
H,
2) haloalkyl,
3) C₁-C₆ alkyl,
4) C₂-C₆ alkenyl,
5) C₂-C₄ alkynyl,
6) C₃-C₇ cycloalkyl,
7) C₃-C₇ cycloalkenyl,
8) aryl,
9) heteroaryl,
10) heterocyclyl,
11) heterobicyclyl,
12) C(O)R¹¹,
13) C(O)Y—R¹¹, or
14) S(O)₂—R¹¹,
wherein the alkyl, alkenyl, alkynyl, cycloalkyl, cycloalkenyl is optionally substituted with one or more R⁶ substituents; and wherein the aryl, heteroaryl, heterocyclyl, and heterobicyclyl is optionally substituted with one or more R¹⁰ substituents;
or R⁸ and R⁹ together with the nitrogen atom to which they are bonded form a five, six or seven membered heterocyclic ring optionally substituted with one or more R⁶ substituents;
R¹⁰ is
halogen,
NO₂,
CN,
B(OR¹³)(OR¹⁴),
C₁-C₆ alkyl,
C₂-C₆ alkenyl,
C₂-C₄ alkynyl,
C₃-C₇ cycloalkyl,
C₃-C₇ cycloalkenyl,
haloalkyl,
OR⁷,
NR⁸R⁹,
SR⁷,
COR⁷,
C(O)OR⁷,
S(O)ₘR⁷,
CONR⁸R⁹,
S(O)₂NR⁸R⁹,
aryl,
heteroaryl,
heterocyclyl, or
heterobicyclyl,
wherein the alkyl, alkenyl, alkynyl, cycloalkyl, and cycloalkenyl is optionally substituted with one or more R⁶ substituents;
R¹¹ is
haloalkyl,
C₁-C₆ alkyl,
C₂-C₆ alkenyl,
C₂-C₄ alkynyl,
C₃-C₇ cycloalkyl,
C₃-C₇ cycloalkenyl,
aryl,
heteroaryl,
heterocyclyl, or
heterobicyclyl,
wherein the alkyl, alkenyl, alkynyl, cycloalkyl, cycloalkenyl is optionally substituted with one or more R⁶ substituents; and wherein the aryl, heteroaryl, heterocyclyl, and heterobicyclyl is optionally substituted with one or more R¹⁰ substituents;
R¹² is
haloalkyl,
C₁-C₆ alkyl,
C₂-C₆ alkenyl,
C₂-C₄ alkynyl,
C₃-C₇ cycloalkyl,
C₃-C₇ cycloalkenyl,
aryl,
heteroaryl,
heterocyclyl,
heterobicyclyl,
C(O)—R¹¹,
C(O)O—R¹¹,
C(O)NR⁸R⁹,
S(O)ₘ—R¹¹, or
C(═Y)NR⁸R⁹,
wherein the alkyl, alkenyl, alkynyl, cycloalkyl, cycloalkenyl is optionally substituted with one or more R⁶ substituents; and wherein the aryl, heteroaryl, heterocyclyl, and heterobicyclyl is optionally substituted with one or more R¹⁰ substituents;
R¹³ and R¹⁴ are each independently
1) H, or
2) C₁-C₆ alkyl; or
R¹³ and R¹⁴ are combined to form a heterocyclic ring or a heterobicyclyl ring;
R³ is H or methyl; and
A and A¹ are both CH₂ or C═O.

33. A process for producing compounds of Formula 8(ii) above, the process comprising:
a) deprotecting PG¹ and PG² from intermediate 8(i), according to claim 30: 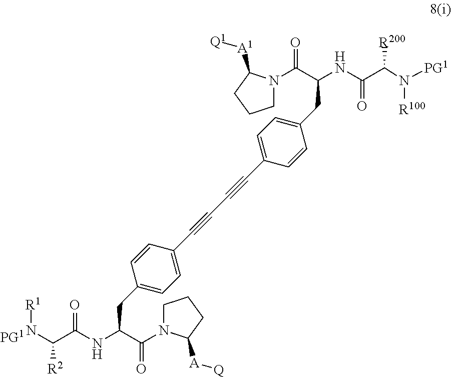
so as to produce compounds of Formula 8(iii): 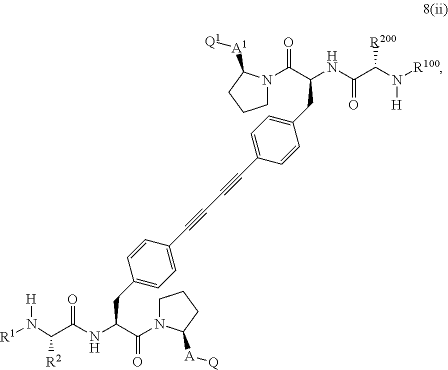
wherein PG¹ and PG² are protecting groups, and wherein
R¹, R¹⁰⁰, R² and R²⁰⁰ are independently selected from:
1) H, or
2) C₁-C₆ alkyl optionally substituted with one or more R⁶ substituents;
Q and Q¹ are each independently
1) NR⁴R⁵,
2) OR¹¹, or
3) S(O)ₘR¹¹; or
Q and Q¹ are each independently 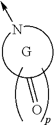
wherein G is a 5, 6 or 7 membered ring which optionally incorporates one or more heteroatoms chosen from S, N or O, the ring being optionally substituted with one or more R¹² substituents;
R⁴ and R⁵ are each independently
H,
haloalkyl,
←C₁-C₆ alkyl,
←C₂-C₆ alkenyl,
←C₂-C₄ alkynyl,
←C₃-C₇ cycloalkyl,
←C₃-C₇ cycloalkenyl,
←aryl,
←heteroaryl,
←heterocyclyl,
←heterobicyclyl,
←C(O)—R¹¹,
←C(O)O—R¹¹,
←C(═Y)NR⁸R⁹, or
←S(O)₂—R¹¹,
wherein the alkyl, alkenyl, alkynyl, cycloalkyl, cycloalkenyl is optionally substituted with one or more R⁶ substituents; and wherein the aryl, heteroaryl, heterocyclyl, and heterobicyclyl is optionally substituted with one or more R¹⁰ substituents;
R⁶ is
halogen,
NO₂,
CN,
haloalkyl,
C₁-C₆ alkyl,
C₂-C₆ alkenyl,
C₂-C₄ alkynyl,
C₃-C₇ cycloalkyl,
C₃-C₇ cycloalkenyl,
aryl,
heteroaryl,
heterocyclyl,
heterobicyclyl,
OR⁷,
S(O)ₘR⁷,
NR⁸R⁹,
NR⁸S(O)₂R¹¹,
COR⁷,
C(O)OR⁷,
CONR⁸R⁹,
S(O)₂NR⁸R⁹
OC(O)R⁷,
OC(O)Y—R¹¹,
SC(O)R⁷, or
NC(Y)NR⁸R⁹,
wherein the aryl, heteroaryl, heterocyclyl, and heterobicyclyl is optionally substituted with one or more R¹⁰ substituents;
R⁷ is
H,
haloalkyl,
C₁-C₆ alkyl,
C₂-C₆ alkenyl,
C₂-C₄ alkynyl,
C₃-C₇ cycloalkyl,
C₃-C₇ cycloalkenyl,
aryl,
heteroaryl,
heterocyclyl,
heterobicyclyl,
R⁸R⁹NC(═Y), or
C₁-C₆ alkyl-C₂-C₄ alkenyl, or
C₁-C₆ alkyl-C₂-C₄ alkynyl,
wherein the alkyl, alkenyl, alkynyl, cycloalkyl, cycloalkenyl is optionally substituted with one or more R⁶ substituents; and wherein the aryl, heteroaryl, heterocyclyl, and heterobicyclyl is optionally substituted with one or more R¹⁰ substituents;
R⁸ and R⁹ are each independently
H,
2) haloalkyl,
3) C₁-C₆ alkyl,
4) C₂-C₆ alkenyl,
5) C₂-C₄ alkynyl,
6) C₃-C₇ cycloalkyl,
7) C₃-C₇ cycloalkenyl,
8) aryl,
9) heteroaryl,
10) heterocyclyl,
11) heterobicyclyl,
12) C(O)R¹¹,
13) C(O)Y—R¹¹, or
14) S(O)₂—R¹¹,
wherein the alkyl, alkenyl, alkynyl, cycloalkyl, cycloalkenyl is optionally substituted with one or more R⁶ substituents; and wherein the aryl, heteroaryl, heterocyclyl, and heterobicyclyl is optionally substituted with one or more R¹⁰ substituents;
or R⁸ and R⁹ together with the nitrogen atom to which they are bonded form a five, six or seven membered heterocyclic ring optionally substituted with one or more R⁶ substituents;
R¹⁰ is
halogen,
NO₂,
CN,
B(OR¹³)(OR¹⁴),
C₁-C₆ alkyl,
C₂-C₆ alkenyl,
C₂-C₄ alkynyl,
C₃-C₇ cycloalkyl,
C₃-C₇ cycloalkenyl,
haloalkyl,
OR⁷,
NR⁸R⁹,
SR⁷,
COR⁷,
C(O)OR⁷,
S(O)ₘR⁷,
CONR⁸R⁹,
S(O)₂NR⁸R⁹,
aryl,
heteroaryl,
heterocyclyl, or
heterobicyclyl,
wherein the alkyl, alkenyl, alkynyl, cycloalkyl, and cycloalkenyl is optionally substituted with one or more R⁶ substituents;
R¹¹ is
haloalkyl,
C₁-C₆ alkyl,
C₂-C₆ alkenyl,
C₂-C₄ alkynyl,
C₃-C₇ cycloalkyl,
C₃-C₇ cycloalkenyl,
aryl,
heteroaryl,
heterocyclyl, or
heterobicyclyl,
wherein the alkyl, alkenyl, alkynyl, cycloalkyl, cycloalkenyl is optionally substituted with one or more R⁶ substituents; and wherein the aryl, heteroaryl, heterocyclyl, and heterobicyclyl is optionally substituted with one or more R¹⁰ substituents;
R¹² is
haloalkyl,
C₁-C₆ alkyl,
C₂-C₆ alkenyl,
C₂-C₄ alkynyl,
C₃-C₇ cycloalkyl,
C₃-C₇ cycloalkenyl,
aryl,
heteroaryl,
heterocyclyl,
heterobicyclyl,
C(O)—R¹¹,
C(O)O—R¹¹,
C(O)NR⁸R⁹,
S(O)ₘ—R¹¹, or
C(═Y)NR⁸R⁹,
wherein the alkyl, alkenyl, alkynyl, cycloalkyl, cycloalkenyl is optionally substituted with one or more R⁶ substituents; and wherein the aryl, heteroaryl, heterocyclyl, and heterobicyclyl is optionally substituted with one or more R¹⁰ substituents;
R¹³ and R¹⁴ are each independently
1) H, or
2) C₁-C₆ alkyl; or
R¹³ and R¹⁴ are combined to form a heterocyclic ring or a heterobicyclyl ring;
R³ is H or methyl; and
A and A¹ are both CH₂ or C═O.

34. A pharmaceutical composition comprising the compound of Formula I, according to claim 1, and a pharmaceutically acceptable carrier, diluent or excipient.

35. A pharmaceutical composition comprising the compound of Formula I, according to claim 1, in combination with any compound that increases the circulating level of one or more death receptor agonists for preventing or treating a proliferative disorder.

36. A method for the preparation of a pharmaceutically acceptable salt of compound of Formula I, according to claim 1, by the treatment of a compound of Formula I with 1 to 2 equiv of a pharmaceutically acceptable acid.

37. A method for the treatment or prevention of a proliferative disorder in a subject, the method comprising: administering to the subject a therapeutically effective amount of the compound or a salt thereof, according to claim 1.

38. The method, according to claim 37, in which the proliferative disorder is cancer.

39. The method, according to claim 37, further comprising administering to the subject a therapeutically effective amount of a chemotherapeutic agent prior to, simultaneously with or after administration of the compound.

40. The method, according to claim 37, further comprising administering to the subject a therapeutically effective amount of a death receptor agonist prior to, simultaneously with or after administration of the compound.

41. The method, according to claim 40, in which the death receptor agonist is TRAIL.

42. The method, according to claim 40, in which the death receptor agonist is a TRAIL antibody.

43. The method, according to claim 40, in which the death receptor agonist is administered in an amount that produces a synergistic effect.

44. The method, according to claim 37, in which the subject is a human.

45. A method for the treatment or prevention of cancer in a subject, the method comprising: administering to the subject a therapeutically effective amount of the pharmaceutical composition, according to claim 34 so as to treat or prevent the cancer.

46. The method, according to claim 45, in which the subject is a human.

47. A method of detecting loss of function or suppression of IAPs in vivo, the method comprising: a) administering to a subject, a therapeutically effective amount of a pharmaceutical composition, according to claim 34; b) isolating a tissue sample from the subject; and c) detecting a loss of function or suppression of IAPs from the sample.

48. The method, according to claim 47, wherein the IAP is XIAP.

49. The method, according to claim 47, wherein the IAP is c-IAP1.

50. The method, according to claim 47, wherein the IAP is c-IAP2.

51. The method, according to claim 47, wherein the tissue is a tumor sample.

52. The method according to claim 47, wherein the tissue is normal tissue.

53. The method, according to claim 47, wherein the tissue is isolated from the hematopeotic system.

54. The method, according to claim 47, wherein mononuclear cells are isolated from blood, and IAP suppression or loss is measured in the mononuclear cells.

55. The method, according to claim 37, wherein the salt of the compound is administered intravenously.


SEQUENCE LISTING (5 sequences)

SEQ ID NO: 1 - protein, 30 aa, human
  VARIANT 28, 29: Xaa = Any Amino Acid or absent
CXXCXXXXXXXXXXXXXXXXHXXXXXXXXC

SEQ ID NO: 2 - protein, 68 aa, human
  VARIANT 13, 16, 17: Xaa = Any Amino Acid or absent
XXXRLXTFXXWPXXXXXXXXXLAXAGFYYXGXXDXVXCFXCXXXXXXWXXXDXXXXXHXXXXPXCXFV

SEQ ID NO: 3 - protein, 46 aa, human
  VARIANT 8: Xaa = Glu or Asp
EXXXXXXXXXXCKXCMXXXXXXXFXPCGHXXXCXXCAXXXXXCPXC

SEQ ID NO: 4 - DNA, 28 bp, human
ttaataggatccatcaacggcttttatc

SEQ ID NO: 5 - DNA, 19 bp, human
gctgcatgtgtgtcagagg